(19) United States
(12) Patent Application Publication
(10) Pub. No.: US 2006/0019940 A1
(43) Pub. Date: Jan. 26, 2006

(54) Novel benzoxazocines and their therapeutic use

(76) Inventors: Andrew Douglas Baxter, Essex (GB); Andrea Walmsley, Essex (GB); Elena Lasterra, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/156,969

(22) Filed: Jun. 20, 2005

Related U.S. Application Data
(63) Continuation-in-part of application No. PCT/GB03/05708, filed on Dec. 22, 2003.

(30) Foreign Application Priority Data
Dec. 20, 2002 (GB) 0229743.0
Apr. 21, 2004 (GB) 0408862.1
Apr. 21, 2004 (GB) 0408863.9

(51) Int. Cl. C07D 491/02; A61K 31/33

(52) U.S. Cl. 514/183; 540/450

(57) ABSTRACT

Compounds of the general formula (1), wherein one of W, X, Y and Z is N or CR⁴ and the others are each CH; and R⁴ is a specified substituent. These compounds inhibit monocamine reuptake, and are useful in the treatment of pain, emesis depression, post traumatic stress disorders, attention deficit disorders, obsessive compulsive disorders, pre-menstrual syndrome, substance abuse and sexual dysfunction.

### REFERENCE TO RELATED APPLICATIONS

This Application is a continuation-in-part of International Patent Applications Nos. PCT/GB03/05708, filed Dec. 22, 2003, and PCT/GB2005/001519, filed Apr. 21, 2005.

### FIELD OF THE INVENTION

This invention relates to novel benzoxazocine compounds which may inhibit monoamine reuptake, and their therapeutic use.

### BACKGROUND OF THE INVENTION

Nefopam [(±)-3,4,5,6-tetrahydro-5-methyl-1-phenyl-1H-2,5-benzoxazocine hydrochloride] is a centrally acting non-narcotic analgesic not structurally related to other analgesics. Nefopam has been shown to induce antinociception in animal models of pain and in humans (reviewed in Heel et al., 1980). However, nefopam is not active in the mouse tail-flick test, or the hot plate test and the Randall-Selitto pressure test in rats (Conway and Mitchell, 1977) suggesting that its analgesic mechanism is not opiate-like or anti-inflammatory in nature. Nefopam's antinociception is not blocked by nalaxone further suggesting that its analgesic action is not through opiate receptors. Although the precise mechanism of antinociception is not known it is thought to involve inhibition of synaptosomal uptake of dopamine, norepinephrine and serotonin (VonVoigtlander et al., 1983; Rosland and Hole, 1990; Mather et al., 2001). Previous in vitro and in vivo studies with nefopam enantiomers have shown that (+)-nefopam has more potent analgesic and dopamine, norepinephrine and serotonin uptake inhibitory properties than (−)-nefopam with the order of potency given as (+)-nefopam>(±)-nefopam>(−)-nefopam (Fasmer et al., 1987; Rosland and Hole, 1990; Mather et al., 2001).

WO03/092689 discloses that the single enantiomers of nefopam are useful for the treatment of pain and emesis.

### SUMMARY OF THE INVENTION

According to a first aspect of this invention, novel compounds are of general formula (1): 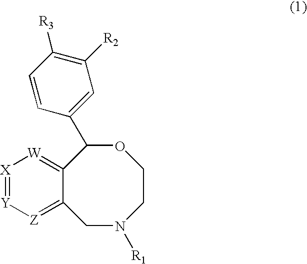
wherein

R₁ is H, C₁-C₆alkyl, optionally substituted with F or C₃-C₆cycloalkyl or C₂-C₆alkenyl;

either R₂ and R₃ are the same or different and are H, a halogen, CN, CF₃, C₁-C₆alkyl or OR₁, or R₂ and R₃ form a five or six membered ring which may be carbocyclic, heterocyclic (containing 1-2 heteroatoms taken from O, N or S), aromatic (such as in naphthalene for example), heteroaromatic (containing 1-2 heteroatoms taken from O, such as in benzofuran for example, N as in quinoline, isoquinoline and quinazoline for example); and

W, X, Y or Z are each N, CH or CR₄.

The case where W═X═Y=Z=CH is specifically excluded. When W is N or R₄, X═Y=Z=CH; when X is N or R₄, W═Y=Z=CH; when Y is N or R₄, W═X=Z=CH; and when Z is N or R₄, W═X═Y═CH.

R₄ is halogen, CF₃, CN, OR₇, SO₂N(R6)₂ (where each R₆is the same or different), COR₆, CO₂R₆, CON(R6)₂ (where R₆ maybe the same or different), NR₁COR₅, NR₁SO₂R₅, NR₁CO₂R₅, NR₁CON(R₆)₂ (where each R₆ is the same or different), OC₁-C₆ alkyl optionally substituted with R₄, C₁-C₆ alkyl optionally substituted with R₄, C₃-C₆cycloalkyl optionally substituted with R₄, C₂-C₆alkenyl optionally substituted with R₄, C₂-C₆alkynyl optionally substituted with R₄, and aryl optionally substituted with R₄. R₄ may also be a five or six membered aromatic heterocycle containing 1-4 heteroatoms selected from N (such as in pyrrole, pyridine, diazoles, diazines, triazoles, triazines or tetrazoles for example) and O (such as in furan, oxazoles, isoxazoles or oxadiazoles for example). Such rings can be linked either through carbon or nitrogen.

R₅ is C₁-C₆ alkyl, C₂-C₆ alkenyl, C₂-C₆ alkynyl, C₃-C₀ cycloalkyl, aryl or heteroaryl.

R₆ is H, C₁-C₆alkyl, C₂-C₆alkenyl, C₂-C₆ alkynyl, C₃-C₆cycloalkyl, aryl or heteroaryl.

R₇ is aryl or heteroaryl.

Novel compounds according to a second aspect of this invention are of general formula (1A): 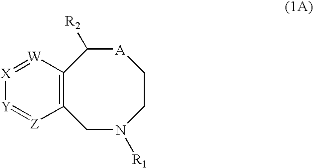
wherein R₁ is H, C₁-C₆ alkyl optionally substituted with F or C₃-C₆ cycloalkyl or C₂-C₄ alkenyl;

A is O, CH₂ or S(O)ₙ where n is 0-2;

one of W, X, Y and Z is N, CH or CR₃ and the others are CH;

R₂ is C₅-C₆ heteroaryl, C₅-C₁₀ cycloalkyl or cycloalkenyl optionally containing one or more heteroatoms selected from O, N and S(O)ₙ where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);

R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;

R₄ is C₁-C₆alkyl, C₂-C₆alkenyl, C₂-C₆ alkynyl, C₃-C₆cycloalkyl, aryl and heteroaryl; and

R₅ is H, C₁-C₆alkyl, C₂-C₆alkenyl, C₂-C₆ alkynyl, C₃-C₆cycloalkyl, aryl or heteroaryl and is the same as or different to another R₅.

Novel compounds according to a third aspect of this invention are of general formula (1B) 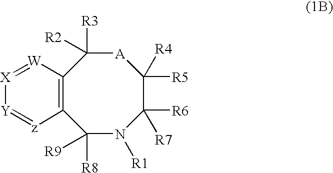
wherein:

A may be O, CH2, S, S(O)n; where n is an integer 0-2;

R₁ may be H, C₁-C₆ alkyl, optionally substituted with F or C₃-C₆ cycloalkyl or C₂-C₄ alkenyl; W, X, Y or Z may be N, CH or CR₁₀; when W is N or CR₁₀, X═Y=Z=CH; when X is N or R₁₀, W═Y=Z=CH; when Y is N or CR₁₀, W═X=*Z=CH; and when Z is N or CR₁₀, W═X═Y═CH;

R₂ may be C₅-C₆ heteroaryl, C₅-C₁₀ cycloalkyl or cycloalkenyl, optionally containing one or more heteroatom taken from the list O, N and S(O)n; where n is an integer 0-2, and optionally substituted with R₁₁;

R₃, R₄, R₅, R₆, R₇, R₈, and R₉ may be the same or different taken from H and C₁₋₆alkyl, but specifically excludes the compound where all are equal to H;

R₁₀ is represented by a group taken from the list of halogen; CF₃, CN; OR₁₃; SO₂N(R₁₃)₂ (where each R₁₃ maybe the same or different); COR₁₃; CO₂R₁₃; CON(R₁₃)₂ (where R₁₃ maybe the same or different); NR₁COR₁₂; NR₁SO₂R₁₂; NR₁CO₂R₁₂; NR₁CON(R₁₃)₂ (where each R₁₃ maybe the same or different) or can be OC₁-C₆ alkyl substituted with R₁₀, C₁-C₆ alkyl optionally substituted with R₁₀, C3-C₆ cycloalkyl optionally substituted with R₁₀, C₂-C₆ alkenyl optionally substituted with R₁₀, C₂-C₆ alkynyl optionally substituted with R₁₀ and aryl optionally substituted with R₁₀. R₁₀ may also be a five or six membered aromatic heterocycle containing 1-4 heteroatoms taken from N (such as in pyrrole, pyridine, diazoles, diazines, triazoles, triazines or tetrazoles for example) and O (such as in furan, oxazoles, isoxazoles or oxadiazoles for example). Such 2 0 rings can be linked either through carbon or nitrogen;

R₁₁ may be a halogen, CN, CF₃, C₁-C₆ alkyl or OR₁. R₁₁ may also be part of a fused five or six membered ring which may be carbocyclic, heterocyclic (containing 1-2 heteroatoms taken from O, N or S), aromatic, heteroaromatic (containing 1-2 heteroatoms taken from O or N );

R₁₂ can be C₁-C₆ alkyl, C₂-C₆ alkenyl, C₂-C₆ alkynyl, C₃-C₆ cycloalkyl, aryl and heteroaryl, and

R₁₃ can be H, C₁-C₆alkyl, C₂-C₆ alkenyl, C₂-C₆ alkynyl, C₃-C₆ cycloalkyl, aryl and heteroaryl.

According to a further aspect of this invention, novel compounds are of general formula (1C) 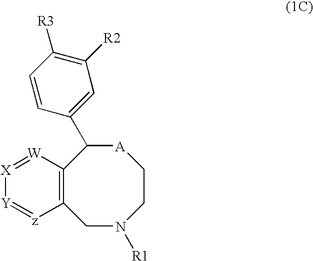
wherein:

A may be CH₂, S, S(O)n; where n is an integer 0-2;

R₁ may be H, C₁-C₆ alkyl, optionally substituted with F or C₃-C₆ cycloalkyl or C₂-C₄ alkenyl;

either R₂ and R₃ may be the same or different and may be H, a halogen, CN, CF₃, C₁-C₆ alkyl or OR₁, or R₂ end R₃ may form a five or six membered ring which may be carbocyclic, heterocyclic (containing 1-2 heteroatoms taken from O, N or S), aromatic (such as naphthalene for example), heteroaromatic (containing 1-2 heteroatoms taken from O, such as benzofuran for example, N as in quinoline, isoquinoline and quinazoline for example); and

W, X, Y or Z are each N, CH or CR₄.

The case where W═X═Y=Z=CH is specifically excluded. When W is N or CR₄, X═Y=Z=CH; when X is N or CR₄, W═Y=Z=CH; when Y is N or CR₄, W═X=*Z=CH; and when Z is N or R₄, W═X═Y═CH.

R₄ is halogen, CF₃, CN; OR₇; SO₂N(R₆)₂ (where each R₆ is the same or different); COR₆; CO₂R₆; CON(R₆)₂ (where N maybe the same or different); NR₁COR₅; NR₁SO₂R₅ ; NR₁CO₂R₅; NR₁CON(R₆)₂ (where each R₆ is the same or different), OC₁-C₆ alkyl substituted with R₄, C₁-C₆ alkyl optionally substituted with R₄, C₃-C₆ cycloalkyl optionally substituted with R₄, C₂-C₆ alkenyl optionally substituted with R₄, C₂-C₆ alkynyl optionally substituted with R₄ and aryl optionally substituted with R₄ R₄ may also be a five or six membered aromatic heterocycle containing 1-4 heteroatoms taken from N (such as in pyrrole, pyridine, diazoles, diazines, triazoles, triazines or tetrazoles for example) and O (such as in furan, oxazoles, isoxazoles or oxadiazoles for example). Such rings can be linked either through carbon or nitrogen.

R₅ can be C₁-C₆ alkyl, C₂-C₆ alkenyl, C₂-C₆ alkynyl, C₃-C₆ cycloalkyl, aryl and heteroaryl.

R₆ can be H, C₁-C₆ alkyl, C₂-C₆ alkenyl, C₂-C₆ alkynyl, C₃-C₆ cycloalkyl, aryl and heteroaryl.

R₇ is aryl or heteroaryl.

Salts, solvents, polymorphs and all isomeric forms of these compounds are included within the scope of the invention.

Compounds of the invention are useful as therapeutic agents. Further, in compounds of formula (1), those wherein R₄is a halogen atom such as Br are useful as intermediates.

### DESCRIPTION OF PREFERRED EMBODIMENTS

It will be appreciated that the compounds according to the invention contain an asymmetrically substituted carbon atom. The presence of this asymmetric centre can give rise to stereoisomers, and in each case the invention is to be understood to extend to all such stereoisomers, including enantiomers and diastereomers, and mixtures including racemic and non-racemic mixtures thereof.

As used in this specification, alone or in combination, the term “C₁-C₆ alkyl” refers to straight or branched chain alkyl moiety having from one to six carbon atoms, including for example, methyl, ethyl, propyl, isopropyl, butyl, tert-butyl, pentyl, hexyl and the like.

The term “C₂-C₆ alkenyl” refers to a straight or branched chain alkyl moiety having two to six carbon atoms and having in addition one double bond, of either E or Z stereochemistry where applicable. This term would include for example, vinyl, 1-propenyl, 1-and 2- butenyl, 2- methyl-2-propenyl etc.

The term “C₂-C₆ alkynyl” refers to a straight or branched chain alkyl moiety having two to six carbon atoms and having in addition one triple bond. This term would include for example, ethynyl, 1-propargyl, 1- and 2- butynyl etc.

The term “C₃-C₆ cycloalkyl” refers to a saturated alicyclic moiety having from three to six carbon atoms and includes for example cyclopropyl, cyclobutyl, cyclopentyl, cyclohexyl and the like.

The term “aryl” means an optionally substituted phenyl or naphthyl group.

The term “carbocyclic” refers to a saturated alicyclic moiety having five or six carbon atoms and includes for example benzofused cyclopentyl and cyclohexyl and the like.

The term “heterocyclic” refers to a saturated heterocyclic moiety having from five or six atoms but containing one or more heteroatom from the group N, O and S includes for example benzofused pyrrolidinyl, tetrahydrofuranyl, piperidinyl, dioxalane and the like.

The term “heteroaromatic” refers to aromatic ring systems of five or six atoms or which at least one atom is selected from the group O, N and S and includes for example benzofused furanyl, thiophenyl, pyridyl, indolyl, pyridazinyl, piperazinyl, pyrimidinyl and the like.

The term “halogen” means fluorine, chlorine, bromine or iodine.

Compounds of the invention may be prepared by any suitable method known in the art and/or by the processes described below. It will be appreciated that where a particular stereoisomer of formula (1) is required, the synthetic processes described herein may be used with the appropriate homochiral starting material and/or isomers maybe resolved from mixtures using conventional separation techniques (e.g. HPLC).

Compounds according to the invention may be prepared by the following process. In the description and formulae below, R groups and other variables are as defined above, except where otherwise indicated. It will be appreciated that functional groups, such as amino, hydroxyl or carboxyl groups, present in the various compounds described below, and which it is desired to retain, may need to be in protected form before any reaction is initiated. In such instances, removal of the protecting group may be the final step in a particular reaction. Suitable protecting groups for such functionality will be apparent to those skilled in the art. For specific details see “Protective Groups in Organic Synthesis”, Wiley Interscience, T W Greene, P G M Wuts.

A process for preparing compounds of general formula (1A), where W, X, Y or Z is N or C—Br and A is O or S(O)ₙ, comprises cyclisation with acid (for instance with p-toluenesulphonic acid) of a diol (2) which can in turn be obtained by reduction of ketone (3) with a suitable reducing agent.

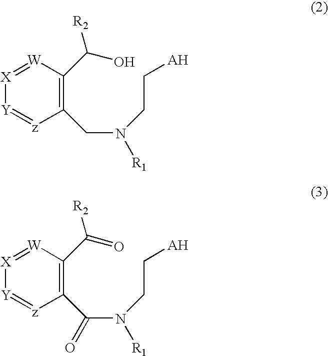

Reduction of a keto amide of general formula (3) can be carried out with reagents well known to those familiar in the art of synthetic organic chemistry. An example of a highly reactive reducing agent is lithium aluminium hydride, although reagents based on borane (e.g. borane tetrahydrofuran complex) or modified sodium borohydride reduction (e.g. with a nickel or cobalt salt enhancer) may be equally effective.

Equally, reduction of the ketone in (3), for example with sodium borohydride, followed by acid cyclisation, for example with p-toluenesulphonic acid, then ultimate reduction of the amide group, for example with borane, also leads to compounds of general formula (1).

Ketones (3) can be prepared by condensation of a carboxylic acid (4) or an active derivative thereof, with an amine (5). Active derivatives of acids of formula (4) include, for example, acid anhydrides or acid halides, such as acid chlorides.

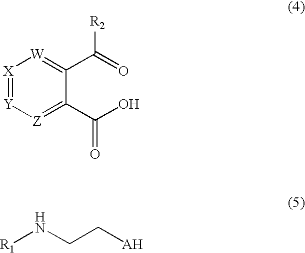

The coupling reaction may be performed using standard conditions for amidation reactions of this type. Thus, the reaction may be achieved in a solvent, for example an inert organic solvent such as an ether, e.g. a cyclic ether such as tetrahydrofuran, an amide, e.g. a substituted amide such as dimethylformamide, or a halogenated hydrocarbon such as dichloromethane at low temperature, e.g. −30° C. to ambient temperature, such as −20° C. to 0° C., optionally in the presence of as base, e.g. an organic base such as an amine, e.g. triethylamine or a cyclic amine such as N-methylmorpholine. Where an acid (4) is used directly, the reaction may additionally be performed in the presence of a condensing agent, for example a diimide such as N,N′-dicyclohexylcarbodiimide, advantageously in the presence of a triazole such as 1-hydroxybenzotriazole. Alternatively, the acid may be reacted with a chloroformate, for example ethyl chloroformate, prior to reaction with the amine (5).

Acids (4) may be prepared by Friedel-Crafts acylation of R₂ with an anhydride (7). This reaction is carried out in an inert solvent (such as dichloromethane) in the presence of a Lewis acid catalyst (such as aluminium trichloride).

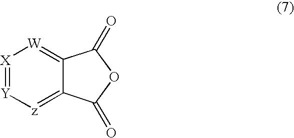

It is well recognised by those skilled in the art that such reactions may provide mixtures of products and in turn that these mixtures can often be separated by flash column chromatography. For example, where Y═C—Br, W═X=Z=CH and R₂ is phenyl, Friedel-Crafts acylation under aluminium trichloride catalysis provides two isomeric bromides (4a) and (4b). These can be readily separated by column chromatography and independently progressed to compounds of general formula (1), wherein X or Y Is C—Br, by the route described above.

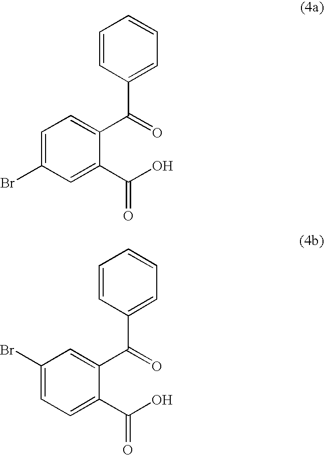

Compounds of general formula (1) where one of W, X, Y and Z is C-hal such as C—Br represent flexible intermediates that may be used for the preparation of other compounds of general formula (1). For instance, such compounds can be smoothly converted into the corresponding nitrile (R₃═CN) either by reaction with cuprous cyanide in a dipolar aprotic solvent such as N-methylpyrrolidinone (NMP) or under palladium-catalysed conditions.

The nitrile can be readily converted, by hydrolysis, into the primary amides (R₃═CONH₂), esters and the corresponding carboxylic acids (CO₂R₅) or into the corresponding tetrazoles, by treatment with a suitable azide donor such as sodium azide or trimethylsilylazide.

A specific example of such conversions is shown in the following scheme:

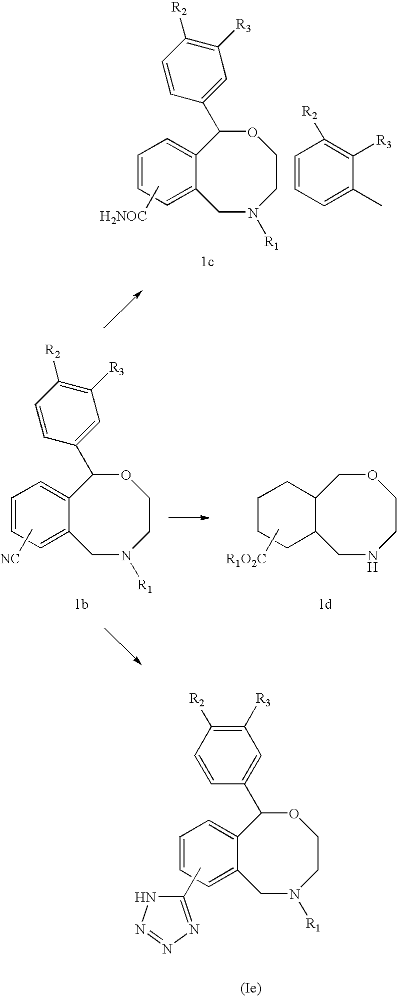

In addition, compounds of general formula (1A) where one of W, X, Y and Z is C-hal such as C—Br can be lithiated with n-, sec- or tert-butyllithium in an inert organic solvent such as an ether, e.g. a cyclic ether such as tetrahydrofuran, at very low temperature, e.g. −78° C. Treatment with either a carbon (e.g. carbon dioxide, N,N-dimethylformamide or paraformaldehyde), sulphur (e.g. SO₂Cl₂, followed by amidation, such as with ammonia) or nitrogen (diphenylphosphoryl azide, followed by reduction, such as with REDAL) provides access, by subsequent derivatisation, to derivatives where R₃ is CO₂R₅, CON(R₅), CH₂OR₅, SO₂N(R₄)₂, NR₁COR₄, NR₁CO₂R₅ or NR₁CON(R₅)₂.

In addition, compounds of general formula (1) where one or W, X, Y and Z is C-hal such as C—Br can undergo palladium-catalysed coupling reactions with carbon-based coupling partners. Thus, such compounds can be coupled to alkenes of the general type CH₂═CHR₃ under Heck conditions, to alkynes of the general type CH═CHR, under Sonogoshira conditions, or to metalloheterocycles, e.g. where the metal is tin, under Stille coupling conditions. This gives access to compounds where R₃ is optionally substituted C₂-C₆ alkenyl or C₂-C₆ alkynyl, or a five-membered aromatic heterocycle containing 1-4 heteroatoms selected from N (such as in pyrrole, diazoles, triazoles or tetrazoles) and O (such as in furan, oxazoles or oxadiazoles). Such coupling reactions ensure that chains and rings are linked through carbon.

In addition to the examples described above, additional compounds of formula (1) may be prepared by interconversion of other compounds of formula (1). Thus, for example, a compound wherein R₃ is alkyl may be prepared by hydrogenation (using palladium on carbon in a suitable solvent, such as an alcohol, e.g. ethanol) of a corresponding compound wherein R₃ is alkenyl.

While the procedures decribed relate to production of compounds of formula (1A), they can readily be adapted to prepare other compounds of the invention, as will be apparent to one of ordinary skill in the art.

Any mixtures of final products or intermediates obtained can be separated on the basis of the physico-chemical differences of the constituents, in known manner, into the pure final products or intermediates, for example by chromatography, distillation, fractional crystallization, or by formation of a salt if appropriate or possible under the circumstances.

The compounds according to the invention exhibit in vitro inhibiting activities with respect to monoamine (i.e. noradrenaline, serotonin and dopamine) reuptake. The activity and selectivity of the compounds may be determined by use of an appropriate monoamine reuptake assay.

This invention also relates to a method of treatment for patients (including man and/or mammalian animals raised in the dairy, meat or fur industries or as pets) suffering from disorders or diseases which can be attributed to monoamine reuptake as previously described, and more specifically, a method of treatment involving the administration of a compound of the invention.

Accordingly, compounds of the invention can be used among other things in the treatment of pain and emesis but also may find utility in a range of other therapeutic indications such as depression, post traumatic stress disorders, attention deficit disorders, obsessive compulsive disorders, pre-menstrual syndrome, substance abuse and sexual dysfunction;

a method of management (by which is meant treatment of prophylaxis) of disease or conditions mediated by monoamine reuptake in mammals, in particular in humans, which method comprises administering to the mammal an effective, amount of a compound of the invention above, or a pharmaceutically acceptable salt thereof;

and a compound of the invention for use in human or veterinary medicine, particularly in the management (by which is meant treatment or prophylaxis) of diseases or conditions mediated by monoamine reuptake;

and the use of a compound of the invention in the preparation of an agent for the management (by which is meant treatment or prophylaxis) of diseases or conditions mediated by monoamine reuptake.

The disease or conditions referred to above include pain, emesis depression, post traumatic stress disorders, attention deficit disorders, obsessive compulsive disorders, pre-menstrual syndrome, substance abuse and sexual dysfunction.

Compounds of the invention may be administered orally, topically, buccally, ocularly, rectally, vaginally, parenterally, intra-nasally, sublingually or by inhalation spray, e.g. in dosage unit formulations containing non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats etc, the compounds of the invention are effective in the treatment of humans.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. The composition may be in immediate or controlled release form.

Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyeryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules where in the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate polyvinyl-pyrrolidone, gum tragacanth and gum acacia; dispersing or wetting agents may be a naturally occuring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters dervied from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example gycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc containing a compound of the invention are employed. For the purposes of this application, topical application includes mouth washes and gargles.

Dosage levels of the order of from about 0.05 mg to about 140 mg per kilogram of body weight per day are useful in the treatment of the above-indicated conditions (about 2.5 mg to about 7 g per patient per day). For example, emesis may be effectively treated by the administration of from about 0.01 to 50 mg of the compound per kilogram of body weight per day (about 0.5 mg to about 3.5 g per patient per day).

The amount of active ingredient that may be combined with the carrier materials to produce a single dosage form will vary depending upon the host treated and the particular mode of administration. For example, a formulation intended for the oral administration of humans may vary from about 5 to about 95 percent of the total composition. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy.

The following Examples illustrate the invention

### 2-Benzoyl-4-bromobenzoic acid (2a) and 2-Benzoyl-5-bromobenzoic acid (2b)

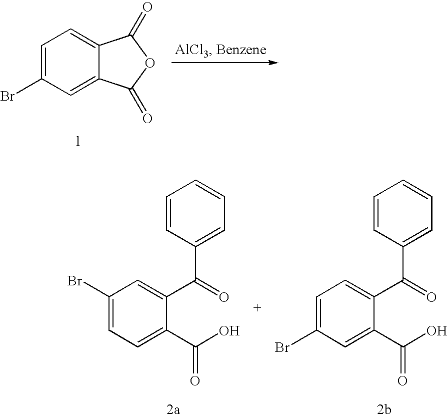

A mixture of 4-bromophthalic anhydride (1) (8.6 g, 37.9 mmol) and aluminium chloride (10 g, 75 mmol, 2 equiv.) were heated under reflux for six hours under an atmosphere of nitrogen. The hot reaction mixture was poured into a solution of water:conc. hydrochloric acid (9:1, 200 mL) and the aqueous layer extracted with dichloromethane (2×150 mL). The organic extract was dried over magnesium sulphate, filtered and evaporated under reduced pressure to furnish the crude product as an off white solid, The solid was dissolved in ethanol (80 mL) and water added until the solution remained turbid. The mixture was allowed to stand at room temperature for four hours; the precipitate formed was filtered, washed with hexane (2×10 mL) and dried under suction to furnish compound 2a. Yield 4.1 g, 35%.

¹H nmr (250 MHz, CDCl₃); 7.94 (1H, d, J 8.0, C Hₐᵣ), 7.71 (3H, m, C Hₐᵣ), 7.58(1H, t, J 7.5, C Hₐᵣ), 7.51 (1H, d, J 1.5 CHₐᵣ), 7.44 (2H, t, J7.5, CHₐᵣ), 7.0-6.4 (1H, bs, OH).

The mother liquors were concentrated under reduced pressure to half the volume and stirred at room temperature. The precipitate formed was filtered, washed with hexane (2×10 mL) and dried under suction to provide 2b. Yield 4.8 g, 42%

¹H nmr (250 MHz, CDCl₃); 8.70-8.30 (1H, bs, OH), 8.20 (1H, d, J 1.5, CHₐᵣ), 7.80 (2H, dd, J 8.0, 1.5, CHₐᵣ), 7.71 (2H, m, CHₐᵣ), 7.58 (1H, m, CHₐᵣ), 7.44 (2H, m, CHₐᵣ), 7.26 (1H, d, J 8.0 CHₐᵣ).

### N-(2-hydroxyethyl)-N-methyl-2-benzoyl-4-bromobenzamide (3a)

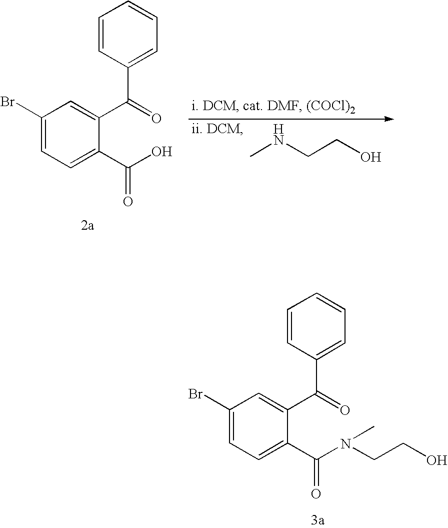

A solution of 2M oxalyl chloride (3.69 mL, 7.38 mmol, 1.1 equiv.) in dichloromethane was added dropwise to a suspension of compound 2a in dichloromethane (12 mL) and catalytic N,N-dimethylformamide (2 drops) at room temperature under a nitrogen atmosphere. Gas evolution was rapid and as the reaction proceeded the solid dissolved in the dichloromethane. After 2.5 hours the solvent was removed under reduced pressure and the resulting solid co-evaporated with dichloromethane (2×20 mL) to remove traces of excess oxalyl chloride. The crude acid chloride was dissolved in dichloromethane (15 mL) and added dropwise to a solution of N-methylaminoethanol (593 μL, 7.38 mmol, 1.1 equiv.) and triethylamine, (1.03 mL, 7.38 mmol, 1.1 equiv) in dichloromethane (15 mL) cooled to 0° C. in an ice bath. The resulting solution was stirred at room temperature for 3 hours, quenched with saturated aqueous sodium dicarbonate (20 mL) and separated. The aqueous layer was extracted with dichioromethane (30 mL) and the combined organic fractions washed with brine (20 mL), dried over magnesium sulfate and filtered. The solvent was removed under reduced pressure and the crude product pruified by column chromatography, eluting with ethyl acetate: hexane (4:1), followed by ethyl acetate (100%) as product eluted. Compound 3a was furnished as a semi solid. Yield 2.23 g, 92%. The product exists are a mixture of rotomers in a 3-2 ratio.

¹H nmr (250 MHz, CDCl₃); 7.85 (2H, m, CHₐᵣ), 7.75-7.62 (3H, m, CHₐᵣ), 7.34(0.6H, J8.0, CHₐᵣ), 7.33 (0.4H, J8.0, CHₐᵣ), 3.83 (1.2H, t, J4.5, CH₂OH), 3.76 (0.8H, t, J4.5, CH₂OH), 3.59 (2H, t, J4.5, NCH₂), 3.08 (0.4H, s, NCH₃) 2.99 (0.6H, s, NCH₃), 2.45 (1H, bs, OH).

### N-(2-hydroxyethyl)-N-methyl-2-benzoyl-5-bromobenzamide (3b)

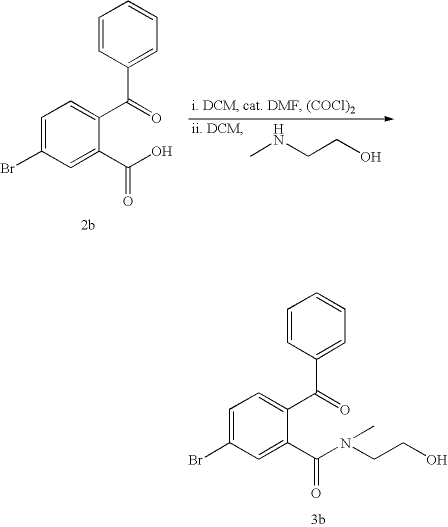

A solution of oxalyl chloride (6.5 mL, 74.3 mmol, 1.1 equiv.) in dichloromethane (150 mL) was added dropwise to a suspension of compound 2a (20.6 g, 67.5 mmol, ratio of 3:1 of 2a:2b respectively) in dichloromethane (50 mL) and catalytic N,N-dimethylformamide (4 drops) at room temperature under a nitrogen atmosphere. Gas evolution was rapid and as the reaction proceeded the solid dissolved in the dichloromethane. After 2.5 hours the solvent was removed under reduced pressure and the resulting solid co-evaporated with dichloromethane (2×50 mL) to remove traces of excess oxalyl chloride. The crude acid chloride was dissolved in dichloromethane (100 mL) and added dropwise to a solution of N-methylaminoethanol (6 mL, 74,3 mmol, 1.1 equiv.) and triethylamine, (10.4 mL, 74.3 mmol, 1.1 equiv) in dichloromethane (150 mL) cooled to 0° C. in an Ice bath. The resulting solution was stirred at room temperature for 3 hours, quenched with saturated brine (100 mL) and separated. The aqueous layer was extracted with dichloromethane (2×50 mL) and the combined organic fractions washed with brine (100 mL), dried over magnesium sulfate and filtered. The solvent was removed under reduced pressure and the crude product purified by column chromatography, eluting with ethyl acetate:hexane (4:1) to elute compound 3b. Yield 6.2 g, 25%. Eluting with ethyl acetate (100%) furnished compound 3a as a semi solid, Yield 14.6 g, 60%. The product exists are a mixture of rotomers in a 3:2 ratio.

¹H nmr (250 MHz, CDCl₃); 7.78(1.8H, m, CHₐᵣ), 7.60 (2.7H, m, CHₐᵣ), 7.49 (2.5H, CHₐᵣ), 7.45 (0.4H, d, J 8.5, CHₐᵣ), 7.37 (0.6H, d, J 8.5, CHₐᵣ), 3.86 (1.2H, m, CH₂OH), 3.76 (0.8H, m, CH₂OH), 3.61 (2H, m, NCH₂), 3.08 (0.4H, s, NCH₃), 3.00 (0.6H, s, NCH₃).

### N-(2-hydroxyethyl)-N-methyl-4-bromo-2-(1-hydroxy-1-phenyl)methyl benzylamine (4a)

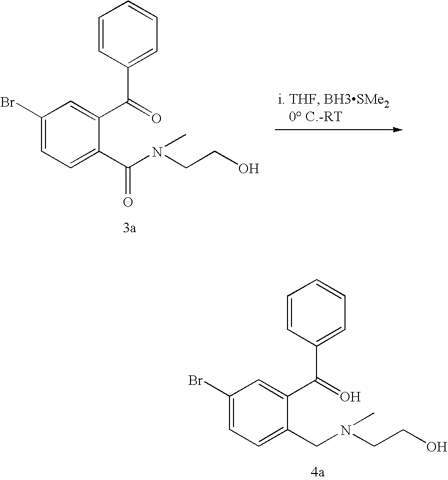

Amide 3a (13.8 g, 38 mmol) was dissolved in tetrahydrofuran (75 mL) and cooled to 0° C. in an ice bath. A 2M solution of borane dimethylsulfide complex (84 mL, 168 mmol, 4.4 equiv.) was added dropwise and the resulting solution stirred at room temperature for 17 hours. The reaction was carefully quenched with 6M hydrochloric acid solution (84 mL) and the resulting solution heated under reflux for 1 hour. Tetrahydrofuran was removed under reduced pressure and remaining solution diluted with water (70 mL) and extracted with diethyl ether (2×100 mL). The aqueous layer was basified with 3.75M sodium hydroxide solution and extracted with ethyl acetate (2×200 mL). The combined ethyl acetate extracts were dried over magnesium sulfate, filtered and evaporated under reduced pressure to furnish the desired product 4a as a colourless glass. Yield 10.2 g, 77%.

¹H nmr (250 MHz, CDCl₃); 7.38-7.27 (7H, m, CHₐᵣ), 7.08 (1H, d, J 80, CHₐᵣ), 5.83 (1H, s, CHOH), 3.70-3.65 (2H, m, OCH₂), 3.41 (1H, d, J 12.5, ArCHₐH_{b}N), 3.29(1 H, d, J 12.5, ArCHₐH_{b}N), 2.58-2.55 (2H, m, NCH₂), 2.21 (3H, s, NCH₃).

### N-(2-hydroxyethyl)-N-methyl-5-bromo-2-(1-hydroxy-1-phenyl)methyl benzylamine (4b)

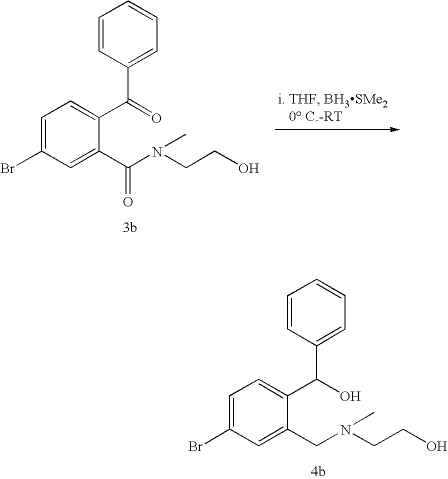

Amide 3b (7.4 g, 20.4 mmol) was dissolved in tetrahydrofuran (40 mL) and cooled to 0° C. in an ice bath. A 2M solution of borane dimethylsulfide complex (45 mL, 9 ommol, 4.4 equiv.) was added dropwise and the resulting solution stirred at room temperature for 17 hours. The reaction was carefully quenched with 6M hydrochloric acid solution (45 mL) and the resulting solution heated under reflux for 1 hour. Tetrahydrofuran was removed under reduced pressure and the remaining solution was diluted with water (45 mL) and extracted with diethyl ether (3×50 mL). The aqueous layer was basified with 3.75M sodium hydroxide solution and extracted with ethyl acetate (2×100 mL). The combined ethyl acetate extracts were dried over magnesium sulfate, filtered and evaporated under reduced pressure to furnish the desired product 4b as a colourless glass. Yield 6.5 g, 91%.

¹H nmr (250MHz, CDCl₃); 7.41-7.27 (7H, m, CHₐᵣ), 7.06 (1H, d, J 8.0, CHₐᵣ), 5.86 (1H, s, CHOH), 3.72-3.66 (2H, m, OCH₂), 3.44 (1H, d, J 12.5, ArCHₐH_{b}N), 3.32 (1H, d, J 12.5, ArCHₐH_{b}N), 2.59 (2H, m, NCH₂), 2.23 (3H, s, NCH₃).

### EXAMPLE 1

### 9-Bromo-5-methyl-1-phenyl -1,3,4,6-tetrahydro-5H-benz[f]-2,5-oxazocine (5a)

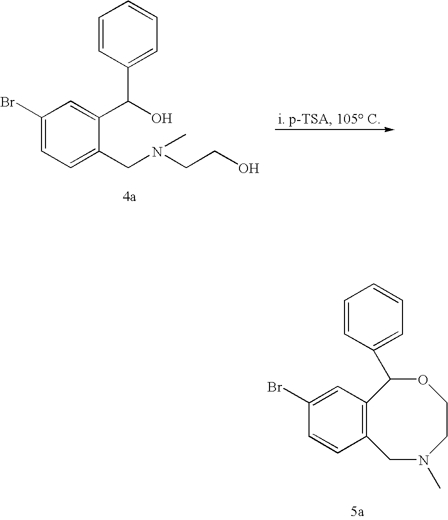

Diol 4a (14.6 g, 41.6 mmol) was dissolved in toluene (115 mL) and pare-toluenesulfonic acid monohydrate (11.9 g, 62.4 mmol, 1.5 equiv.) added. The toluene was removed under reduced pressure and the resulting oil heated at 105° C. for 4 hours. On cooling the oil was suspended in water (100 mL) and basified with 3.75M sodium hydroxide solution. The aqueous layer was extracted with ethyl acetate (2×200 mL), dried over magnesium sulfate, filtered and evaporated under reduced pressure to furnish the product 5a as pale brown oil. Yield 9.25 g, 67%.

¹H nmr(250 MHz, CDCl₃); 7.38-7.25 (5H, m, CHₐᵣ), 7.11 (2H, t, J 8.0, CHₐᵣ), 5.72 (1H, s, CHO), 4.82 (1H, d, J 13.0, ArCH₁H_{b}), 4.19 (1H, dt, J 3.0, 8.0, OCHₐH_{b}), 3.82 (1H, ddd, J 3.0, 6.0, 13.0, OCHₐH_{b}), 3.62 (1H, d, J 13.0,ArCHₐH_{b}), 2.81 (1H, m NCHₐH_{b}), 2.61 (1H, ddd, J 3.0, 8.0, 13.0, NCHₐH_{b}), 2.43(3H, 5, CH₃).

### EXAMPLE 2

### 8-Bromo-5-methyl-I-phenyl-I,3,4,6-tetrahydro-5H-benz(f]-2,5-oxazocine (5b)

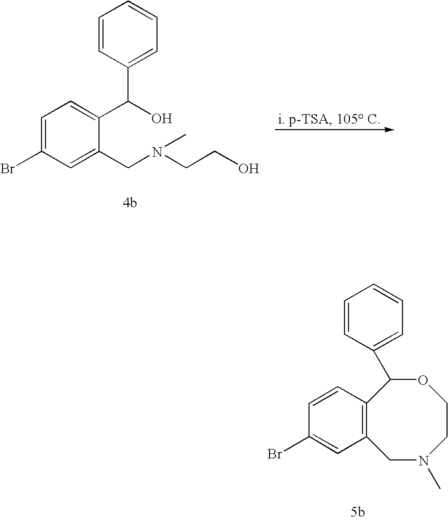

Diol 4b (6.5 g, 18.6 mmol) was dissolved in toluene (50 mL) and para-toluenesulfonic acid monohydrate (5.3 g, 27.8 mmol, 1.5 equiv.) added. The toluene was removed under reduced pressure and the resulting oil heated at 105° C. for 4 hours. On cooling the oil was dissolved in 3.75M sodium hydroxide solution and extracted with ethyl acetate (2×80 mL), dried over magnesium sulfate, filtered and evaporated under reduced pressure to furnish the crude product. The crude product was purified by dry flash chromatography eluting with ethyl acetate (100%, followed by ethyl acetate:methanol (5%)). Fractions containing product were combined and evaporated under reduced pressure to furnish 5b as pale brown oil. Yield 3.6 g, 58%.

¹H nmr (250 MHz, CDCl₃); 7.37-7.22 (7H, m, CHₐᵣ), 6.86 (1H, d, J 8.0, CHₐᵣ), 5.74 (1H, s, CHO), 4.79(1H, d, J 13.0, ArCHₐH_{b}), 4.16(1H, dt, J 3.0, 8.0, OCHₐH_{b}), 3.85 (1H, ddd, J2.0, 60, 13.0, OCHₐH_{b}), 3.61 (1H, d, J 13.0,ArCHₐH_{b}), 2.81(1H, ddd, J2.0, 8.0, 13.0, NCHₐH_{b}), 2.63 (1H, ddd, J3.0, 6.0, 13.0, NCHₐH_{b}), 2.46 (3H, s, CH₃).

### EXAMPLE 3

### 9-Cyano-5-methyl-I-phenyl-I,3,4,6-tetrahydro-5H-benz[f]-2,5-oxazocine (6a)

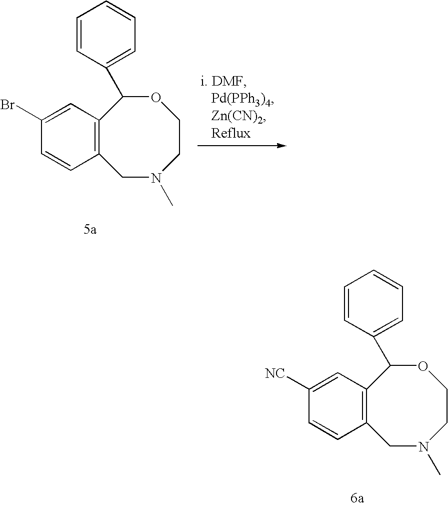

Bromo analogue 5a (0.2 g, 0.6 mmol), Zn(CN)₂ (53 mg, 0.6 mmol), and Pd(PPh₃)₄(34 mg, 0.03 mmol), were dissolved in degassed anhydrous DMF (3 mL) under a N₂atmosphere. The mixture was refluxed under N₂for 24 hours. The mixture was allowed to cool to room temperature, filtered through celite and washed through with DCM (50 ml). The filtrate was then quenched with water (10 ml) and solvent extracted. The organic extract was dried over MgSO₄, filtered and solvent removed under reduced pressure. The crude product was purified by dry flash chromatography eluting with DCM:MeOH (98:2). Fractions containing the product were combined and evaporated under reduced pressure to produce 6a as pale brown oil. Yield 106 mg, 63%.

¹H nmr(250MHz, CDCl₃); 7.51 (1H, dd, J2.0, 8.0, CHₐᵣ), 7.35-7.23(7H, m, CHₐᵣ), 5.79 (1H, s, CHO), 4.90 (1H, d, J 13.0, ArCHₐH_{b}), 4.25-4.16 (1H, m, OCHₐH_{b}), 3.86 (1H, ddd, J3.0, 6.0, 13.0, OHCₐH_{b}) 3.71 (1H, d, J 13.0, ArCHₐH_{b}), 2.78(1H, ddd, J 3.0, 8.0, 13.0, NCHₐH_{b}), 2.68 (1H, ddd, J 3.0, 5.0, 13.0, NCHₐH_{b}), 2.45 (3H, s, CH₃).

### EXAMPLE 4

### 8-Cyano-5-methyl-1-phenyl-I,3,4,6-tetrahydro-5H-benz[f]-2,5-oxazocine (6b)

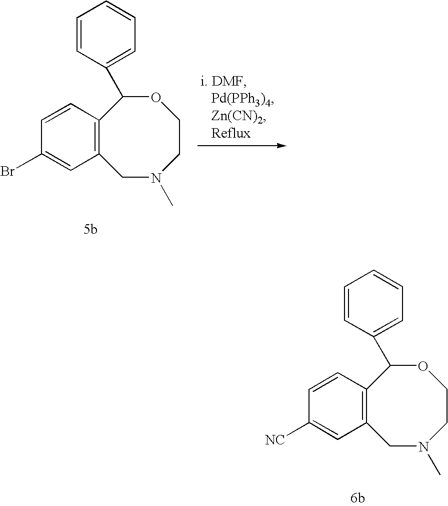

Bromo analogue 5b (0.2 g, 0.6 mmol), Zn(CN)₂ (53 mg, 0.6 mmol), and Pd(PPh₃)₄ (34 mg, 0.03 mmol), were dissolved in degassed anhydrous DMF (3 mL) under a N₂ atmosphere. The mixture was refluxed under N₂ for 24 hours. There is no change in Rf value for compound 6b from the starting material 5b. Completion of the reaction can only be determined by ¹H nmr. The mixture was allowed to cool to room temperature, filtered through celite and washed through with DCM (50 ml). The filtrate was quenched with water (10 ml) and solvent extracted. The organic extract was dried over MgSO₄, filtered and solvent removed under reduced pressure. The crude product was purified by dry flash chromatography eluting with DCM:MeOH (98:2). Fractions containing the product were combined and evaporated under reduced pressure to produce 6b as pale brown oil. Yield 110 mg, 66%.

¹H nmr (250MHz, CDCl₃); 7.46 (2H, td, J 2.0. 8.0, CHₐᵣ), 7.31 (5H, m, CHₐᵣ), 7.10 (1H, d, J 8.0, CH,), 5.76 (1 H, s, CHO), 4.97 (1H, d, J 13.0, ArCHₐH_{b}), 4.25 (1H, ddd, J 3.0, 8.0, 13.0. OCHₐH_{b}), 3.82 (1H, ddd, J 3.0, 5.0, 13.0, ArCHₒH_{b}) 3.62 (1H, d, J 13.0, NCHₐH_{b}), 2.74(1 H, ddd, J 2.0, 8.0, 13.0, NCHₐH_{b}), 2.44 (3H, s, CH₃).

### EXAMPLE 5

### 5-methyl-1-phenyl-1,3,4,6-tetrahydro-5H-benz[f]-2,5-oxazocine-9-carboxamide (7a)

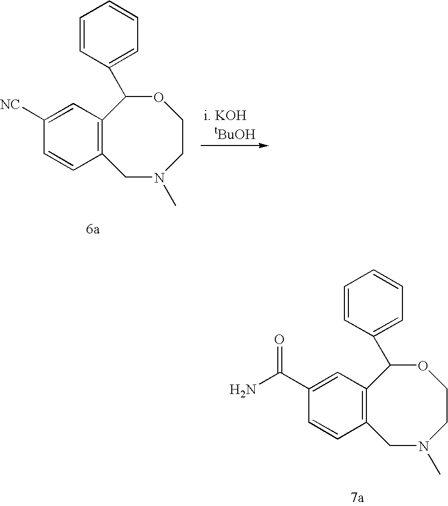

Finely ground potassium hydroxide (130 mg, 2.32 mmol) in tertiary butanol (4 ml) was added to nitrile 6a (169 mg, 0.61 mmol). The solution was heated under reflux for 1.5 hours with stirring. On cooling, the reaction was diluted with brine (12.5 ml) and extracted with chloroform (3×10 ml). The combined organic layers were washed with brine (2×5 ml), dried over potassium carbonate and concentrated under reduced pressure to yield a yellow solid. Flash column chromatography (15%-25% methanol in ethyl acetate) furnished amide 7b as a cream coloured solid. Yield 195 mg, quantitative yield.

IR (vₘₐₓ/cm⁻¹)1656;

¹H nmr (250 MHz, CD₃OD); 7.75 (1H, dd, J 7.9, 1.8, CHₐᵣ), 7.60 (1H, d, J 1.5, CHₐᵣ), 7.36 (1H, d, J 8.0, CHₐᵣ), 7.28 (5H, brs, CHₐᵣ), 5.86 (1H, s, CHO), 5.02 (1H, d, J 12.5, ArCHₐCH_{b}), 4.22 (1H, m, OCHₐH_{b}), 3.88 (1H, dq, J 12.7, 4.7, 2.5, OCHₐH_{b}), 3.72 (1H, d, J 12.5, ArCHₐCH_{b}), 2.76 (1H, m, NCHₐH_{b}), 2.56 (1H, dq, J 14.2, 3.1, NCHₐH_{b}), 2.43 (3H, s, NCH₃).

### EXAMPLE 6

### 5-methyl-1-phenyl-1,3,4,6-tetrahydro-5H-benz[f]-2,5-oxazocine-8-carboxamide (7b)

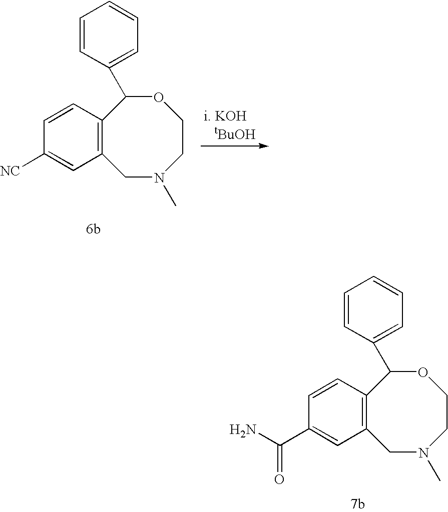

Finely ground potassium hydroxide (87 mg, 1.55 mmol) in tertiary butanol (4 ml) was added to nitrile 6b (135 mg, 0.49 mmol). The solution was heated under reflux for 1.5 hours with stirring. On cooling, the reaction was diluted with brine (12.5 ml) and extracted with chloroform (3×10 ml). The combined organic layers were washed with brine (2×5 ml), dried over potassium carbonate and concentrated under reduced pressure to yield a yellow solid. Flash column chromatography (15%-25% methanol in ethyl acetate) furnished amido 7b as a cream coloured solid. Yield 140 mg, 97%.

¹H nmr (250 MHz, CD₃OD); 7.74-7.63 (2H, m, CHₐᵣ), 7.25-7.09 (6H, m, CHₐᵣ), 5.80 (1H, s, CHO), 4.96 (1H, d, J 10.1, ArCHₐCH_{b}), 4.25 (1H, d, J 10.0, ArCHₐCH_{b}), 3.89-3.65 (2H, m, OCHₐH_{b}), 2.83 (1H, m, NCHₐH_{b}), 2.66 (1H, m, NCHₐH_{b}), 2.53 (3H, s, NCH₃).

### EXAMPLE 7

### N-(1,1,1-trimethylmethoxycarbonyl)-5-methyl-I-phenyl-1,3,4,6-tetrahydro5H-benz[f]-2,5-oxazocine-9-methylamine (8a)

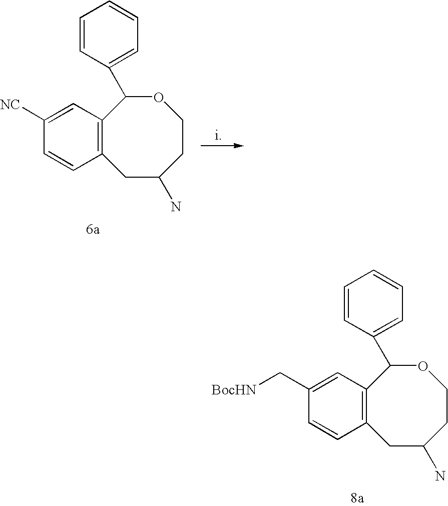

Sodium borohydride (250 mg, 6.61 mmol) was cautiously added to a solution of nickel chloride (122 mg, 0.94 mmol), Boc₂O (412 mg, 1.89 mmol) and nitrile 6a (260 mg, 0.94 mmol) in anhydrous methanol (10 ml) at 0° C. Once the vigorous initial reaction had subsided, the mixture was left to stir overnight under a nitrogen atmosphere at room temperature. The methanol was removed under reduced pressure and the resulting precipitate dissolved in ethyl acetate. Saturated aqueous sodium bicarbonate was added, the mixture sonicated and the resulting precipitate filtered. The organic layer was separated from the aqueous which was extracted with ethyl acetate (2×10 mL). The combined organic layers were dried over sodium sulfate and concentrated under reduced pressure to yield a brown oil. Flash column chromatography (5%-10% methanol in dichloromethane) furnished protected amine 8a as an orange/brown oil. Yield 141 mg, 39%.

¹H nmr (250 MHz, CDCl₃); 7.29-7.15 (7H, m, CHₐᵣ), 6.88 (H, s, CHₐᵣ), 5.74 (1H, s, CHO), 4.79 (1H, d, J 13, ArCHₐCH_{b}), 4.19-4.17 (3H, m, CH₂, OCHₐH_{b}), 3.83 (1H, d, J2, 6, 13, ArCHₐCH_{b}), 3.65 (1H, d, J13.0, NCHₐH_{b}) 2.78 (1H, ddd, J 2, 8, 15, OCHₐH_{b}), 2.61 (1H, ddd, J 2, 6, 15, NCHₐH_{b}) 2.43 (3H, S, CH₃), 1.45 (9H, s, Boc, ^{t}Bu).

### EXAMPLE 8

### N-(1,1,1-trimethylmethoxycarbonyl)-5-methyl-1-phenyl-1,3,4,6-tetrahydro5H-benz[f]-2,5-oxazocine-8-methylamine (8b)

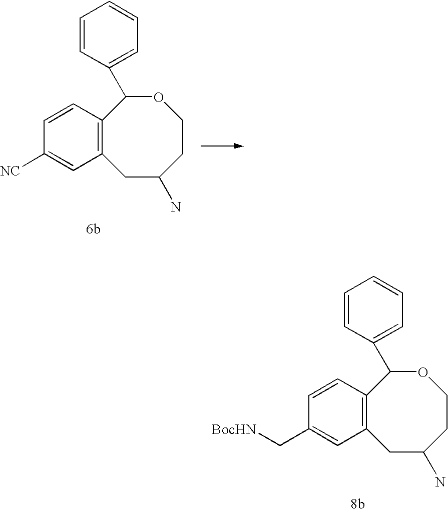

Sodium borohydride (470 mg, 12.6 mmol) was cautiously added to a solution of nickel chloride (460 mg, 3.6 mmol), Boc₂O (780 mg, 3.6 mmol) and nitrile 6a (500 mg, 1.8 mmol) in anhydrous methanol (20 ml) at 0° C. Once the vigorous initial reaction had subsided, the mixture was left to stir overnight under a nitrogen atmosphere at room temperature. The methanol was removed under reduced pressure and the resulting precipitate dissolved in ethyl acetate. Saturated aqueous sodium bicarbonate was added, the mixture sonicated and the resulting precipitate filtered. The organic layer was separated from the aqueous which was extracted with ethyl acetate (2×10 mL). The combined organic layers were dried over sodium sulfate and concentrated under reduced pressure to yield a brown oil. Flash column chromatography (5%-10% methanol in dichloromethane) furnished protected amine 8b as an orange/brown oil. Yield 260 mg, 48%.

¹H nmr (250 MHz, CDCl₃); 7.30-7.16 (7H, m, CHₒᵣ) 6.90 (H, s, CH), 5.74 (1H, s, CHO), 4.83 (1H, d, J 12.8, ArCHₐCH_{b}), 4.77 (H, brs, NH), 4.23 (3H, m, CH₂, OCHₐH_{b}), 3.87 (1H, ddd, J 2, 5, 13, ArCHₐCH_{b}), 3.69 (1H, d, J 13.0, NCHₐH_{b}) 2.81 (1H, m, OCHₐH_{b}) 2.64 (1H, ddd, J 3, 5, 14 NCHₐH_{b}), 2.47 (3H, s, CH₃), 1.42 (9H, s, Boc, ^{t}Bu)

### EXAMPLE 9

### 5-methyl-1,9-diphenyl-1,3,4,6-tetrahydro-5H-benz[f]-2,5-oxazocine (9a)

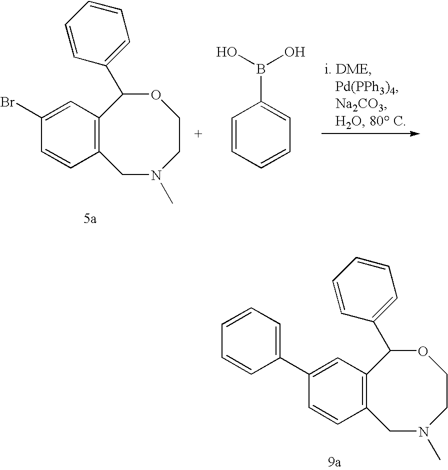

To a solution of 5a (280 mg, 0.84 mmol) in dimethoxyethane (6 mL) was added tetrakis(triphenylphosphine)palladium (105 mg, 10 mol %) and the solution stirred at room temperature for 10 minutes under and atmosphere of nitrogen. A solution of sodium carbonate (536 mg, 5.0 mmol, 6 equiv.) in water (2 mL) was added and the biphasic system stirred for 10 minutes. Phenylboronic acid (103 mg, 1.1 mmol, 1.3 equiv.) was added and the mixture heated at 80° C. for 1.5 hours. The reaction was diluted with dichloromethane (20 mL), washed with water (20 mL) and the organic layer dried over MgSO₄. Filtration and evaporation under reduced pressure furnished a brown oil. Diethyl ether (25 mL) was added and a precipitate formed. After filtration the filtrate was evaporated under reduced pressure and the resulting oil purified by column chromatography, eluting with diethyl ether: methanol (4:1). Fractions containing product were combined, evaporated under reduced pressure to furnish the desired product as a pale yellow oil. Yield 172 mg, 61%.

¹H nmr (250 MHz, CDCl₃); 7.52-7.47 (3H, m, CHₐᵣ), 7.43-7.25 (10H, m, CHₐᵣ), 5.87 (1H, s, CHO), 4.88 (1H, d, J 12.5, ArCHaCH_{b}), 4.24 (1H, m, OCHₐCH_{b}), 3.92 (1H, m, OCHₐCH_{b}), 3.72 (1H, d, J 12.5, ArCHₐCH_{b}), 2.89 (1H, m, NCHₐCH_{b}), 2.68 (1H, m, NCHₐCH_{b}), 2.51 (3H, s, NCH₃).

### EXAMPLE 10

### 5-methyl-1,8-diphenyl-1,3,4,6-tetrahydro-5H-benz[f]-2,5-oxazocine (9b)

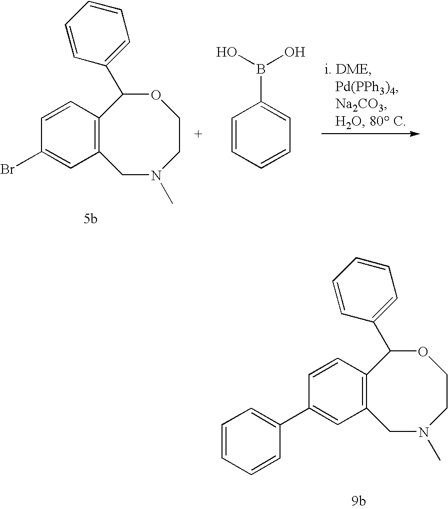

To a three neck flask containing 5b (200 mg, 0.72 mmol) in toluene (4 ml) under a nitrogen atmosphere at room temperature was added tetrakis(triphenylphosphine)palladium (105 mg, 0.08 mmol). After stirring for 10 mins, Caesium carbonate (1.4 g, 4.32 mmol) was added followed by phenylboronic acid (96 mg, 0.93 mmol). The reaction was refluxed at 110° C. for 3 hours. When cooled, the reaction was filtered through celite which was subsequently washed with ethyl acetate. The organic layer was then separated from the aqueous and dried over MgSO₄. The crude compound was concentration under reduced pressure and purified using column chromatography eluted with EtOAc to produce the pure compound as an off white solid. Yield 55 mg, 23%.

¹H nmr (250 MHz, CDCl₃); 7.50 (2H, d, J 7.2, CHₐᵣ), 7.45-7.29 (10H, m, CHₐᵣ), 7.07 (1H, d, J 7.8, CHₐᵣ), 5.84 (1H, s, CHO), 4.90 (1H, d, J 12.8, ArCHₐCH_{b}), 4.23 (1H, td, J 10.5, 2.5, OCHₐCH_{b}), 3.91 (1H, ddd, J 12.5, 5.7, 2.1, OCHₐCH_{b}), 3.79 (1H, d, J 12.7, ArCHₐCH_{b}), 2.90 (1H, ddd, J 14.2, 8.4, 2.1, NCHₐCH_{b}), 2.68 (1H, ddd, J 14.1, 5.6, 2.5, NCHₐCH_{b}), 2.52 (3H, s, NCH₃).

### EXAMPLE 11

### 9-(3,5-dimethylisoxazol-4-yl)-5-methyl-1-phenyl-1,3,4,6-tetrahydro-5H-benz[f]-2,5-oxazocine (10a)

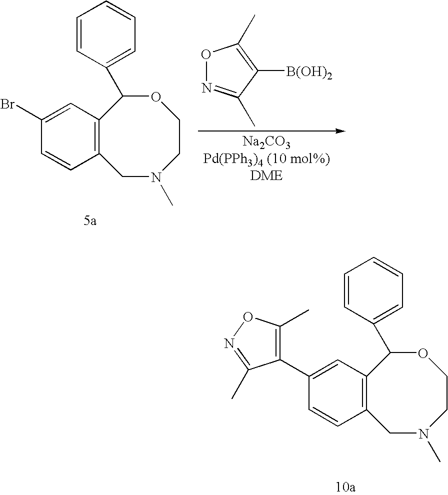

To a three neck flask containing 5a (300 mg, 0.91 mmol) in DME (4 ml) under a nitrogen atmosphere at room temperature was added tetrakis(triphenylphosphine)palladium (105 mg, 0.09 mmol). After stirring for 10 mins, Na₂CO₃ (576 mg, 5.44 mmol) dissolved in water (2 ml) was added followed by 3,5-dimethyl-4-isoxazolylboron ic acid (166 mg, 1.18 mmol). The reaction was refluxed at 85° C. for 2 hours. When cooled, the reaction was filtered through celite which was subsequently washed with ethyl acetate. The organic layer was then separated from the aqueous and dried over MgSO₄ Concentration under reduced pressure and column chromatography (10% MeOH in DCM) gave a brown oil. Yield 304 mg, 97%.

¹H nmr (250 MHz, CDCl₃); 7.28 (6H, m, CHₐᵣ) 7.13 (1H, dd, J 7.6, 1.5, CHₐᵣ), 6.85 (1H, d, J 1.1, CHₐᵣ), 5.84 (1H, s, CHO), 4.76 (1H, d, J 12.8, ArCHₐCH_{b}), 4.19 (1H, m, OCHₐCH_{b}), 3.90 (1H, m, OCHₐCH_{b}), 3.73 (1H, d, J 12.8, ArCHₐCH_{b}), 2.89 (1H, ddd, d, J 14.1, 7.9, 1.8, NCHₐCH_{b}), 2.68 (1H, ddd, J 14.1, 6.1, 2.2, NCHₐCH_{b}), 2.50 (3H, s, NCH₃), 2.26 (3H, s, CH₃), 2.12 (3H, s, CH₃).

### EXAMPLE 12

### 8-(3,5-dimethylisoxazol-4-yl)-5-methyl-1-phenyl-1,3,4,6-tetrahydro-5H-benz[f]-2,5-oxazocine (10b)

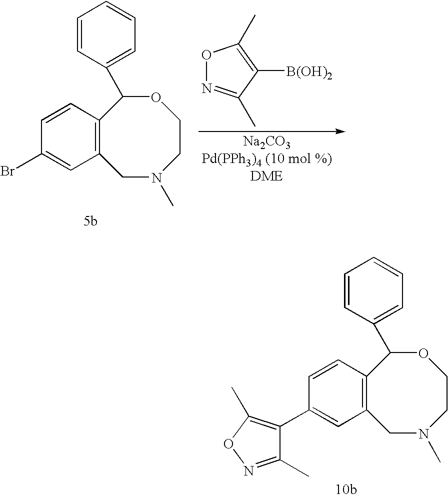

To a three neck flask containing 5b (382 mg, 1.15 mmol) in DME (5 ml) under a nitrogen atmosphere at room temperature was added tetrakis(triphenylphosphine)palladium (133 mg, 0.12 mmol). After stirring for 10 mins, Na₂CO₃ (734 mg, 6.92 mmol) dissolved in water (2 ml) was added followed by 3,5-dimethyl-4-isoxazolylboron ic acid (212 mg, 1.5 mmol). The reaction was refluxed at 85° C. for 2 hours. When cooled, the reaction was filtered through celite which was subsequently washed with ethyl acetate. The organic layerwas then separated from the aqueous and dried over MgSO₄. Concentration under reduced pressure and column chromatography (10% MeOH in DCM) gave a brown oil. Yield 269 mg, 67%.

¹H nmr (250 MHz, CDCl₃); 7.30 (5H, m, CHₐᵣ), 7.08 (3H, m, CHₐᵣ), 5.82 (1H, S, CHO), 4.88 (1H, d, J 12.8, ArCHₐCH_{b}), 4.21 (1H, m, OCHₐCH_{b}), 3.89 (1H, ddd, J 12.6, 5.7, 2.2, OCHₐCH_{b}), 3.72 (1H, d, J 13.0, ArCHₐCH_{b}), 2.86 (1H, ddd, d, J 14.2, 8.3, 2.2, NCHₐCH_{b}), 2.67 (1H, ddd, J 14.2, 5.7, 2.6, NCHₐCH_{b}), 2.47 (3H, s, NCH₃), 2.41 (3H, s, CH₃), 2.27 (3H, s, CH₃).

### EXAMPLE 13

### 2-(5-methyl-I-phenyl-1,3,4,6-tetrahydro-SH-benz[f]-2,5-oxazocine-9-ethenyl)carboxamide (11a)

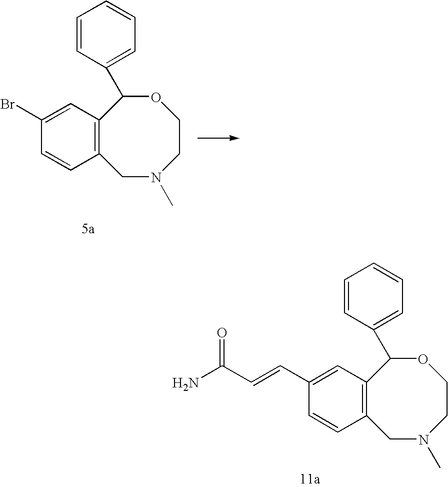

To a sample vial containing a solution of 5a (245 mg, 0.74 mmol), in DMF (3 ml) was added acrylamide (105 mg, 1.47 mmol), followed by Pd(OAc)₂ (17 mg, 0.074 mmol), P(o-tolyl)₃ (64 mg, 0.21 mmol), NaOAc (73 mg, 0.88 mmol), LiCl (63 mg, 1.47 mmol), K₂CO₃ (122 mg, 0.88 mmol), and water (300 μL) sequentially. The reaction vial was sealed and placed in a personal chemistry Microwave, Emrys optimiser at 140° C. for 4 min. On cooling, the compound was purified by column chromatography, gradient elution of DCM; MeOH (5%, 10%, 15% and finally 20%). The compound was isolated as a colourless solid, (138 mg, 58% yield).

¹H nmr (250 MHz, CDCl₃); 7.50 (H, d, J 16.8, CHₐCONH), 7.46-7.21 (7H, m, CHₐᵣ) 7.12 (H, d, J 1.6, CHₐᵣ), 6.34 (H, d, J 16.8, CH_{b}CONH) 5.78 (3H, brs, NH₂, CHO), 4.83 (1H, d, J 13.8, ArCHₐCH_{b}), 4.20 (1H, ddd, J 13.5, 9.2, 2.9 OCHₐCH_{b}), 3.85 (1H, ddd, J 13.5, 5.9, 2.3, OCHₐCH_{b}), 3.72 (1H, d, J 13.8, ArCHₐCH_{b}), 2.82 (1H, ddd, d, J 15.1, 8.9, 2.3, NCHₐCH_{b}), 2.64 (1H, ddd, J 15.4, 6.2, 2.9 NCHₐCH_{b}), 2.46 (3H, s, NCH).

### EXAMPLE 14

### 2-(5-methyl-1-phenyl-1,3,4,6-tetrahydro-5H-benz[f]-2,5-oxazocine-8-ethenyl)carboxamide (11b)

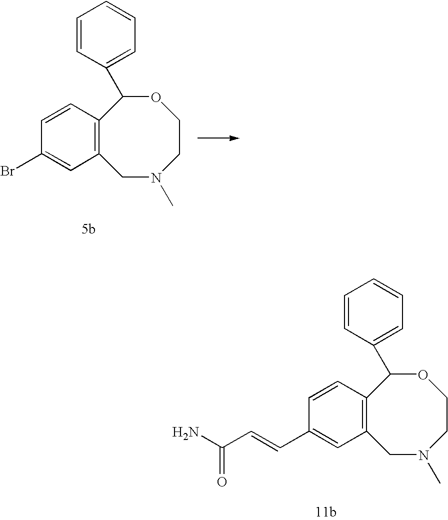

To a sample vial containing a solution of 5b (247 mg, 074 mmol), in DMF (3 ml) was added acrylamide (106 mg, 1.47 mmol), followed by Pd(OAc)₂ (17 mg, 0.07 mmol), P(o-tolyl)₃ (64 mg, 0.21 mmol), NaOAc (73 mg, 0.88 mmol), LiCl (63 mg, 1.47 mmol), K₂CO₃ (122 mg, 0.88 mmol), and water (300 μL) sequentially. The reaction vial was sealed and placed in a personal chemistry Microwave, Emrys optimiser at 140° C. for 4 min. On cooling, the compound was purified by column chromatography, gradient elution of DCM; MeOH (5%, 10%, 15% and finally 20% to elute the product). The compound was isolated as a colourless solid, (51 mg, 21% yield).

¹H nmr (250 MHz, CDCl₃); 7.62 (H, d, J 15.6, CHₐCONH), 7.39-7.26 (7H, m, CHₐᵣ), 7.03 (H, d, J 8.1 CHₐᵣ), 6.46 (H, d, J 15.7, CH_{b}CONH) 5.78 (H, S, CHO), 4.90 (1H, d, J 12.7 ArCHₐCH_{b}), 4.25 (1H, td, J 10.6, 2.7, OCHₐCH_{b}), 3.87 (1H, ddd, J 12.6, 3.5, 2.5, OCHₐCH_{b}), 3.71 (1H, d, J 12.7, ArCHₐCH_{b}), 2.85 (1H, ddd, d, J 14.1, 8.6, 2.2, NCHₐCH_{b}), 2.64 (1H, ddd, J 14.1, 5.1, 2.5, NCHₐCH_{b}), 2.50 (3H, s, NCH₃).

### EXAMPLE 15

### 2-(5-methyl-1-phenyl-1,3,4,6-tetrahydro-5H-benz[f]-2,5-oxazocine-9-ethyl)carboxamide (12a)

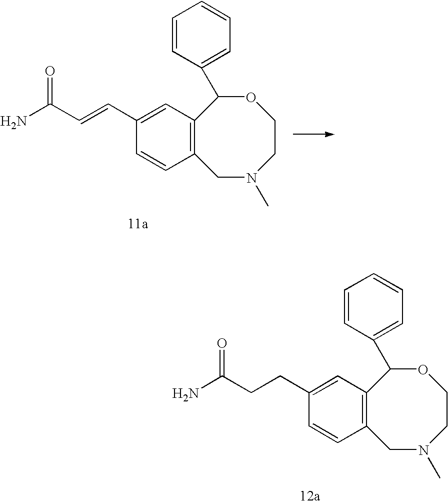

To a round bottom flask containing a solution of 2′-acrylamide-nefopam (130 mg, 0.4 mmol) in methanol (2 ml) was added 20 mg of 10% Pd on charcoal under N₂. On completion of addition, a balloon of H₂ was added and the reaction purged with H₂. The reaction mixture was then left at RT for 24 hours. Once the reaction was completed, the mixture was filtered through a plug of Celite and solvent evaporated. The crude reaction mixture was purified by column chromatography eluted with 10% MeOH:DCM (2 drops of TEA per 200 ml). The compound was isolated as a yellow oil, (80 mg, 62%).

¹H nmr (250 MHz, MeOD); 7.30-7.04 (7H, m, CHₐᵣ), 6.91 (1H, s, CHₐᵣ), 5.75 (H, s, CHO), 4.87 (1H, d, J 13.6 ArCHₐCH_{b}), 4.16 (1H, td, J 9, 3, OCHₐCH_{b}), 3.83 (1H, ddd, J 12.6, 4.7, 2.4, OCHₐCH_{b}), 3.62 (1H, d, J 12.5, ArCHₐCH_{b}), 2.83 (3H, m, d, NCHₐCH_{b}, CH₂), 2.69-2.38 (6H, m, NCHₐCH_{b}, NCH₃, CH₂).

### EXAMPLE 16

### 2-(5-methyl-1-phenyl-1,3,4,6-tetrahydro-5H-benz[f]-2,5-oxazocine-8-ethyl)carboxamide (12b)

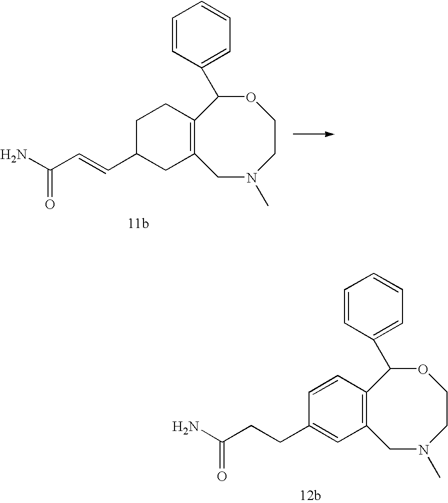

To a round bottom flask containing a solution of 3′-acrylamide-nefopam (96 mg, 0.3 mmol) in methanol (3 ml) was added 15 mg of 10% Pd on charcoal under N₂. On completion of addition, a balloon of H₂ was added and the reaction purged with H₂. The reaction mixture was then left at RT for 24 hours. Once the reaction was completed, the mixture was filtered through a plug of Celite and solvent evaporated. The crude reaction mixture was purified by column chromatography eluted with 10% MeOH:DCM (2 drops of TEA per 200 ml). The compound was isolated as a yellow oil, (96 mg, 100%).

¹H nmr (250 MHz, MeOD); 7.34-7.23 (5H, m, CHₐᵣ), 7.07-7.02 (2H, m, CHₐᵣ), 6.92 (1H, d, J 8, CHₐᵣ), 5.77 (H, s, CHO), 5.59 (2H, brs, NH₂), 4.71 (1H, d, J 12.8, ArCHₐCH_{b}), 4.16 (1H, ddd, J 12.5, 8.3, 2.8 OCHₐCH_{b}), 3.85 (1H, ddd, J 12.5, 5.8, 2.1, OCHₐCH_{b}), 3.65 (1H, d, J 12.5, ArCHₐCH_{b}), 2.93 (2H, t, J 7.3, CH₂), 2.83 (1H, ddd, 14, 7.9, 2.2, NCHₐCH_{b}), 2.60 (H, ddd, J 14, 5.8, 2.45, NCHₐCH_{b}), 2.57-2.48 (5H, m, CH₂, NCH₃, CH₂).

### EXAMPLE 17

### 5-methyl-1-phenyl-1,3,4,6-tetrahydro-5H-benz[f]-2,5-oxazocine-9-methylamine (13a)

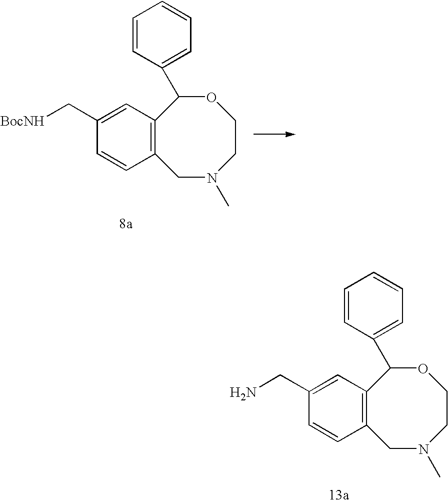

A dry round bottom flask was charged with 8a (70 mg, 0.18 mmol), and TFA (1 ml). The reaction was stirred at RT under N₂ for 1 hour. Once the reaction was completed, the solution was diluted with water (10 ml) and the mixture extracted three times with ether (10 ml). The aqueous portion was base washed with 1M NaOH until PH 14, the aqueous phase was then extracted three times with DCM (20 ml). The organic phase was then dried over MgSO₄ and solvent removed under reduced pressure. The compound was obtained as a yellow oil (25 mg, 60% yield).

¹H nmr (250 MHz, CDCl₃); 7.31-7.18 (7H, m, CHₐᵣ), 6.92 (H, s, CHₐᵣ), 5.76 (H, s, CHO), 4.82 (1H, d, J 13, ArCHₐCH_{b}), 4.19 (1H, td, J8, 3, OCHₐCH_{b}), 3.89-3.65 (4H, m, OCHₐCH_{b}, ArCHₐCH_{b}, CH₂), 2.82 (1H, ddd, d, J 14, 8, 2 NCHₐCH_{b}), 2.60 (1H, ddd, J 14, 5, 2, NCHₐCH_{b}), 2.46 (3H, S, NCH₃), 1.25 (2H, brs, NH₂).

### EXAMPLE 18

### 5-methyl-1-phenyl-1,3,4,6-tetrahydro-5H-benz[f]-2,5-oxazocine-8-methylamine (13b)

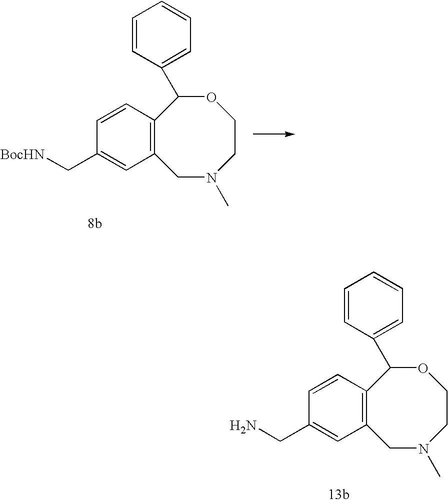

A dry round bottom flask was charged with 8b (60 mg, 0.16 mmol), and TFA (1 ml). The reaction was stirred at RT under N₂ for 1 hour. Once the reaction was completed, the solution was diluted with water (10 ml) and the mixture extracted three times with ether (10 ml). The aqueous portion was base washed with 1M NaOH until PH 14; the aqueous phase was then extracted three times with DCM (20 ml). The organic phase was then dried over MgSO₄ and solvent removed under reduced pressure. The compound was obtained as yellow oil. (26 mg, 60% yield).

¹H nmr (250 MHz, CDCl3); 7.31-7.10 (8H, m, CHₐᵣ), 6.97 (1 H, d, J7.6 CHₐᵣ), 5.79 (H, s, CHO), 4.91 (1H, d, J 12.5, ArCHₐCHb), 4.17 (1H, ddd, J 12.8, 8.3, 2.8, OCHₐCH_{b}), 4.0 (2H, brs, NH2), 3.89-3.83 (3H, m, OCHaCHb, CH₂), 3.67 1H, d, J 12.5, ArCHaCHb), 2.85 (1H, ddd, d, J 14, 8, 2 NCHaCHb), 2.65 (1H, ddd, J 14, 5.7, 2.4, NCHaCHb), 2.49 (3H, S, NCH3).

### 2-(4-Methoxy)benzoyl-4-bromobenzoic acid and 2-(4-methoxy)benzoyl-5-bromobenzoic acid (14)

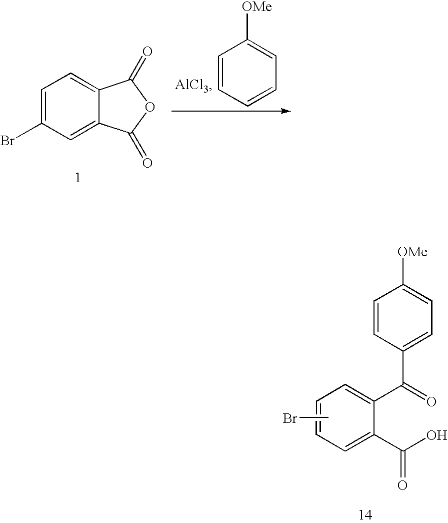

To a flask containing 4-bromophthalic anhydride (1) (25.65 g, 0.11 mol) was added finely crushed aluminium chloride (30.13 g, 0.23 mol). The solid mixture was further crushed and stirred using a spatula. To this mixture was added anisole (85.53 g, 86 m1, 0.79 mol) which initiated the production of HCl gas. Once gas evolution ceased the reaction was heated at 80° C. for 1.5 hours. The hot reaction mixture was poured into a solution of water: conc. hydrochloride acid (9:1, 600 mL) and the aqueous layer extracted with ether (2×150 mL). The organic layer was washed with water and then brine before being dried over magnesium sulphate, filtered and evaporated under reduced pressure to furnish the crude product. Overnight recrystallisation from a mixture of ether (200 ml) and hexane (400 ml) yielded 14, after filtration, a white solid as a mixture of regio isomers. Yield 22.77 g, 60%.

¹H nmr (250 MHz, CDCl₃); 9.28 (2H, brs, 2×CO₂H), 8.19 (1H, d, J 1.8, CHₐᵣ), 7.92 (1H, d, J 8.4, CHₐᵣ),7.78 (1H, d, J 1.8, CHₐᵣ), 7.74 (1H, d, J 1.8, CHₐᵣ), 7.69 (2H, d, J 2.0, CHₐᵣ), 7.65 (2H, d, J 1.8, CHₐᵣ), 7.48 (1H, d, J 1.8, CHₐᵣ), 7.2 (1H, d, J 8.1 CHₐᵣ), 6.91 (2H, d, J 2.5, CHₐᵣ), 6.88 (2H, d, J 2.5, CHₐᵣ), 3.86 (6H, s, 2×CH₃).

### N-(2-hydroxyethyl)-N-methyl-2-(4-methoxy)benzoyl-4-bromobenzamide (15a) and N-(2-hydroxyethyl)-N-methyl-2-(4-methoxy)benzoyl-5-bromobenzamide (15b)

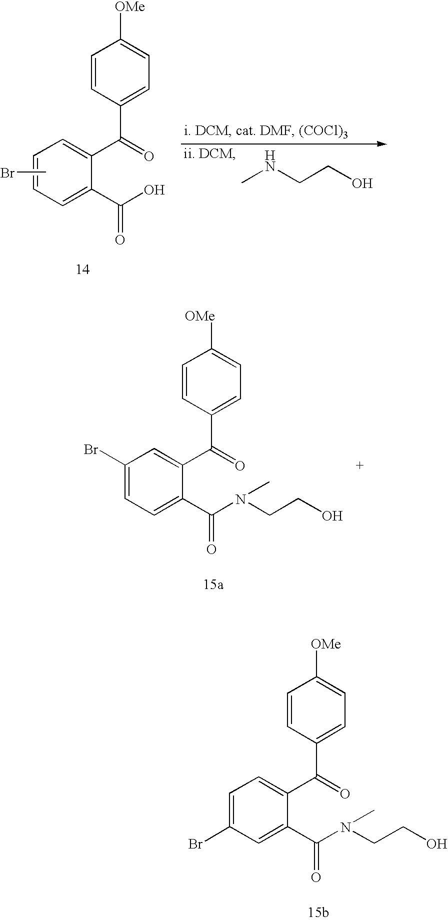

A solution of 2M oxalyl chloride (6.52 mL, 74.73 mmol, 1.1 equiv.) in dichloromethane was added dropwise to a suspension of the mixture of isomers 14 (22.77 g, 67.94 mmol) in dichloromethane (115 mL) and catalytic N,N dimethylformamide (5 drops) at room temperature under a nitrogen atmosphere. Gas evolution was rapid and as the reaction proceeded the solid dissolved in the dichloromethane. After 2.5 hours the solvent was removed under reduced pressure and the resulting solid co-evaporated with dichloromethane (2×100 mL) to remove traces of excess oxalyl chloride. The crude acid chloride was dissolved in dichloromethane (115 mL) and added dropwise to a solution of N-methylaminoethanol (6.0 mL, 74.73 mmol, 1.1 equiv.) and triethylamine, (10.42 mL, 74.73 mmol, 1.1 equiv) in dichloromethane (115 mL) cooled to 0° C. in an ice bath. The resulting solution was stirred at room temperature for 3 hours, quenched with saturated aqueous sodium dicarbonate (100 mL) and separated. The aqueous layer was extracted with dichloromethane (100 mL) and the combined organic fractions washed with brine (50 mL), dried over magnesium sulfate and filtered. The solvent was removed under reduced pressure and the crude product purified by column chromatography, eluting with ethyl acetate: hexane (4:1), followed by ethyl acetate (100%) as product eluted. Compound 15a was furnished as a yellow semi solid. Yield 5.00 g, 19%. The product exists are a mixture of rotomers in a 3:2 ratio.

¹H nmr (250 MHz, CDCl₃); 7.81 (2H, d, J 7.5, CHₐᵣ), 7.71-7.53 (2H, m, CHₐᵣ), 7.31 (1H, m, CHₐᵣ), 6.95 (2H, d, J 7.6, CHₐᵣ), 3.88 (3H, s, OCH₃), 3.80 (2H, t, J 5.1, CH₂OH), 3.72 (1H, m OH), 3.57 (2H, t, J4.9, NCH2), 3.06 (1.2H, S, NCH₃) 2.96 (1.8H, s, NCH₃).

Compound 15b was furnished as a red semi solid. Yield 5.00 g, 19%. The products exist as a mixture of rotomers in a 3:2 ratio.

¹H nmr (250 MHz, CDCl₃); 7.77 (2H, dd, J 9.0,2.4, CHₐᵣ), 7.59-7.53 (2H, m, CHₐᵣ), 7.42-7.31 (1H, m, CHₐᵣ), 6.91 (2H, dd, J 9.0, 2.4, CHₐᵣ), 3.85 (3H, s, OCH), 3.79 (2H, t, J 5.1, CH₂OH), 3.69 (1H, brs OH), 3.56 (2H, t, J 5.1, NCH₂), 3.03 (1.2H, s, NCH₃) 2.95 (1.8H, s, NCH₃).

### N-(2-hydroxyethyl)-N-methyl-4-bromo-2-[1-hydroxy-1-(4-methoxyphenyl)]methylbenzylamine (16a)

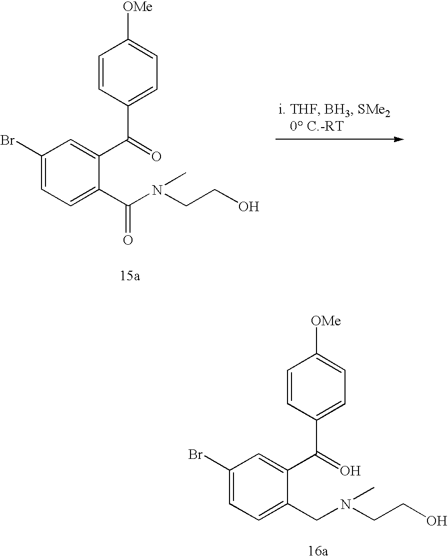

Amide 15a(1.56 g, 3.99 mmol) was dissolved in tetrahydrofuran (10 mL) and cooled to 0° C. in an ice bath. A 2M solution of borane dimethylsulfide complex (8.78 mL, 17.55 mmol, 4.4 equiv.) was added dropwise and the resulting solution stirred at room temperature for 17 hours. The reaction was carefully quenched with 2M hydrochloric acid solution (5 mL) and the resulting solution stirred at room temperature for 2 hours. Tetrahydrofuran was removed under reduced pressure and remaining solution diluted with water (10 mL) and extracted with diethyl ether (2×50 mL). The aqueous layer was basified with 3.75M sodium hydroxide solution and extracted with ethyl acetate (2×50 mL). The combined ethyl acetate extracts were dried over magnesium sulfate, filtered and evaporated under reduced pressure to furnish the desired product 16a as a white foam. Yield 754 mg, 50%.

¹H nmr (250 MHz, CDCl₃); 7.39-7.24 (4H, m, CHₐᵣ), 7.07-6.98 (1H, m, CHₐᵣ), 6.89-6.79 (2H, m, CHₐᵣ), 5.77 (1H, s, CHOH), 3.80 (3H, s, OCH₃), 3.67 (2H, m, OCH₂), 3.41 (1H, d, J, 12.4, ArCHₐH_{b}N), 3.28 (1H, d, J, 12.7, ArCHₐH_{b}N), 2.57-2.50 (2H, m, NCH₂), 2.17 (3H, s, NCH₃).

### N-(2-hydroxyethyl)-N-methyl-5-bromo-2-[1-hydroxy-1-(4-methoxyphenyl)]methylbenzylamine (16b)

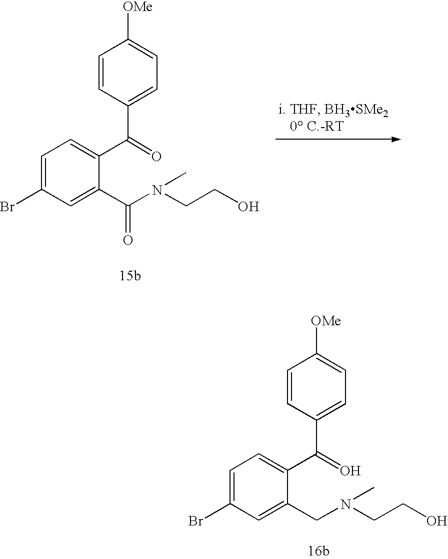

Amide 15b (1.54 g, 3.93 mmol) was dissolved in tetrahydrofuran (10 mL) and cooled to 0° C. in an ice bath. A 2M solution of borane dimethylsulfide complex (8.60 mL, 17.28 mmol, 4.4 equiv.) was added dropwise and the resulting solution stirred at room temperature for 17 hours. The reaction was carefully quenched with 2M hydrochloric acid solution (5 mL) and the resulting solution stirred at room temperature for 2 hours. Tetrahydrofuran was removed under reduced pressure and remaining solution diluted with water (10 mL) and extracted with diethyl ether (2×50 mL). The aqueous layer was basified with 3.75M sodium hydroxide solution and extracted with ethyl acetate (2×5 mL). The combined ethyl acetate extracts were dried over magnesium sulfate, filtered and evaporated under reduced pressure to furnish the desired product 16b as a white foam. Yield 606 mg, 40%.

¹H nmr (250 MHz, CDCl₃); 7.45-7.33 (2H, m, CHₐᵣ), 7.24 (2H, d, J 8.4, CHₐᵣ), 7.03-6.95 (1H, m, CHₐᵣ), 6.86 (2H, d, J 8.7, CHₐᵣ), 5.79 (1H, S, CHOH), 3.79 (3H, s, OCH₃), 3.65 (2H, m, OCH₂), 3.41 (1H, d, J, 12.5, ArCHₐH_{b}N), 3.28 (1H, d, J, 12.5, ArCHₐH_{b}N), 2.56-2.50 (2H, m, NCH₂), 2.18 (3H, s, NCH₃).

### EXAMPLE 19

### 9-Bromo-5-methyl-1-(4-methoxy)phenyl-1,3,4,6-tetrahydro-5H-benz[f]-2,5-oxazocine (17a)

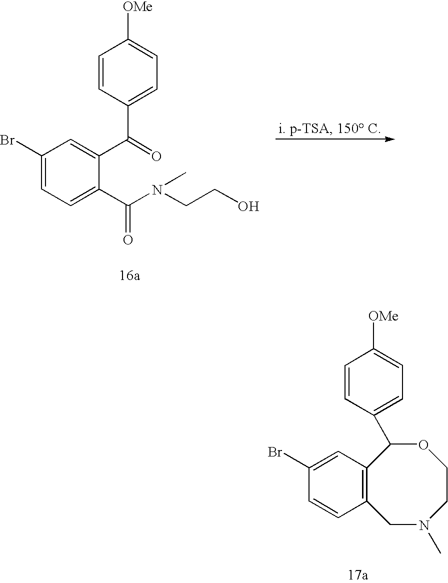 Diol 16a (754 mg, 1.99 mmol) was dissolved in toluene (10 mL) and para-toluenesulfonic acid monohydrate (568 mg, 2.98 mmol, 1.5 equiv.) added. The toluene was removed under reduced pressure and the resulting oil heated at 105° C. for 2 hours. On cooling the oil was suspended in water (10 mL) and basified with 3.75M sodium hydroxide solution. The aqueous layer was extracted with ethyl acetate (2×25 mL), dried over magnesium sulfate, filtered and evaporated under reduced pressure to furnish the crude product which was purified by chromatography eluting with 10% methanol in ethyl acetate. Fractions containing product were combined and evaporated under reduced pressure to furnish 17a. Yield 340 mg, 47%.

¹H nmr (250 MHz, CDCl₃); 7.34 (1H, dd, J 8.1, 1.8, CH,), 7.16 (2H, d, J 8.7, CHₐᵣ), 7.12-7.06 (2H, m, CHₐᵣ), 6.85 (2H, d, J 8.6, CHₐᵣ), 5.66 (1H, s, CHOH), 4.83 (1H, d, J 12.8, ArCHₐH_{b}), 4.15 (1H, m, OCHₐH_{b}), 3.83 (1H, OCHₐH_{b}), 3.78 (3H, s, OCH₃), 3.60 (1H, d, J 12.8, ArCHₐH_{b}), 2.77 (1H, m NCHₐH_{b}), 2.59 (1H, ddd, J 2.6, 5.5, 14.2, NCHₐH_{b}), 2.43 (3H, s, CH₃).

### EXAMPLE 20

### 8-Bromo-5-methyl-1-4-methoxy)phenyl-1,3,4,6-tetrahydro-5H-benz[f]-2,5-oxazocine (17b)

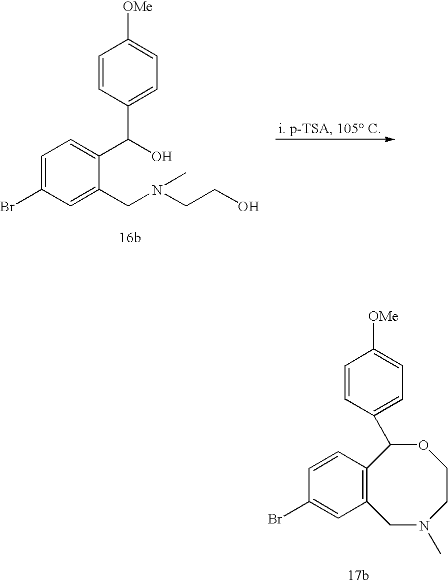

Diol 16b (200 mg, 0.53 mmol) was dissolved in toluene (5 mL) and para-toluenesulfonic acid monohydrate (150 mg, 0.79 mmol, 1.5 equiv.) added. The toluene was removed under reduced pressure and the resulting oil heated at 105° C. for 2 hours. On cooling the oil was suspended in water (10 mL) and basified with 3.75M sodium hydroxide solution. The aqueous layer was extracted with ethyl acetate (2×25 mL), dried over magnesium sulfate, filtered and evaporated under reduced pressure to furnish the crude product which was purified by chromatography eluting with 10% methanol in ethyl acetate. Fractions containing product were combined and evaporated under reduced pressure to furnish 17b as a clear oil. Yield 127 mg, 67%.

¹H nmr (250 MHz, CDCl₃); 7.36 (1H, d, J 1.5, CHₐᵣ), 7.29 (1H, dd, J 8.4, 1.8, CHₐᵣ), 7.15 (2H, d, J 8.7, CHₐᵣ), 6.85 (1H, m, CHₐᵣ), 6.83 (2H, d, J 8.6, CHₐᵣ), 5.68 (1H, s, CHOH), 4.81 (1H, d, J 12.8, ArCHₐH_{b}), 4.13(1H, m, OCHₐH_{b}), 3.83 (1H, OCHₐH_{b}), 3.76 (3H, s, OCH₃), 3.58 (1H, d, J 12.8, ArCHₐH_{b}), 2.77 (1H, m NCHₐH_{b}), 2.61 (1H, ddd, J 2.5, 5.7, 14.1, NCHₐH_{b}), 2.45 (3H, s, CH₃).

### 2-(3-Methoxy)benzoyl-4-bromobenzoic acid and 2-(3-methoxy)benzoyl-5-bromobenzoic acid (18)

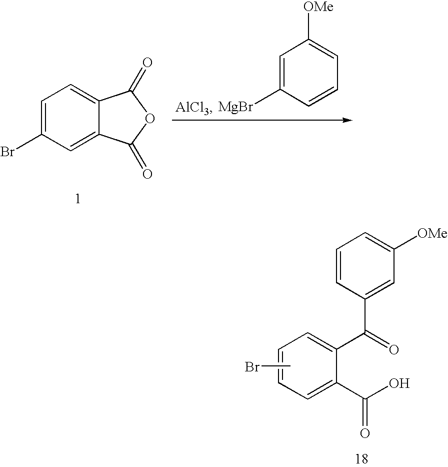

Magnesium (2.45 g, 0.1 mol) was suspended In anhydrous ether (250 ml) under a nitrogen atmosphere at room temperature. 3-Bromoanisole (18.75 g, 0.1 mol) and iodine (cat.) were added to form an initial red solution which became colourless after a few minutes. The reaction was allowed to stir overnight and a pale yellow precipitate formed. The precipitate was added, via a dropping funnel, to a stirred solution of 4-bromophthalic anhydride (1) (25 g, 0.1 mol) in toluene (150 ml) and ether (30 ml) under a nitrogen atmosphere. The subsequent reflux was maintained for 24 hours, cooled and quenched with saturated aqueous NH₄Cl. The aqueous layer was extracted with diethyl ether (3×200 ml), dried over MgSO₄, filtered and concentrated under reduced pressure to give the desired mixture of regioisomers (30.1 g, 82% yield).

¹H nmr (250 MHz, CD₃OD); 8.19 (H, s, CHₐᵣ), 7.92 (H, d, CHₐᵣ), 7.74 (2H, dd, J 23.5, 8.7, CHₐᵣ), 7.51 (H. s, CHₐᵣ), 7.36-7.09 (9H, m, CHₐᵣ), 6.95-6.85 (H, m, CHₐᵣ), 6.52-6.40 (H, m, CHₐᵣ), 2.35 (6H, S, OCH₃).

### N-(2-hydroxyethyl)-N-methyl-2-(3-methoxy)benzoyl-4-bromobenzamide (19a) and N-(2-hydroxyethyl)-N-methyl-2-(3-methoxy)benzoyl-5-bromobenzamide (19b)

A solution of 2M oxalyl chloride (8.65 mL, 0.1 mol) in dichloromethane was added dropwise to a suspension of the mixture of isomers 18 (30.0g, 0.09 mol) in dichloromethane (200 ml) and catalytic N,N-dimethylformamide (1 drop) at room temperature under a nitrogen atmosphere. Gas evolution was rapid and as the reaction proceeded the solid dissolved in the dichloromethane. After 2.5 hours the solvent was removed under reduced pressure and the resulting solid co-evaporated with dichloromethane to remove traces of excess oxalyl chloride. The crude acid chloride was dissolved in dichloromethane (200 mL) and added dropwise to a solution of N-methylaminoethanol (6 mL, 0.1 mol.) and triethyamine (14 mL) in dichloromethane (200 mL) cooled to 0° C. in an ice bath. The resulting solution was stirred at room temperature overnight, quenched with saturated aqueous ammonium chloride and separated. The organic layer was washed with water (2×400 mL), dried over magnesium sulfate and filtered. The solvent was removed under reduced pressure and the crude product purified by chromatography, eluting with hexane:ethyl acetate, (1:1) to yield 19a (8.88 g, 25% yield) and 19b (6.13 g, 18% yield) as viscous oils.

19a:

¹H nmr (250 MHz, CDCl₃); 7.61-7.56 (3H, m, CHₐᵣ), 7.49-7.44 (H, m, CHₐᵣ), 7.39-7.26 (8H, m, CHₐᵣ), 7.17-7.13 (2H, m, CHₐᵣ), 3.86-3.83 (9H, m, OCH₃, CH₂), 3.61 (4H, t, J 5.3, CH₂), 3.09 (3H, s, NCH₃), 2.99 (3H, s, NCH₃), 1.41 (2H, brs, OH).

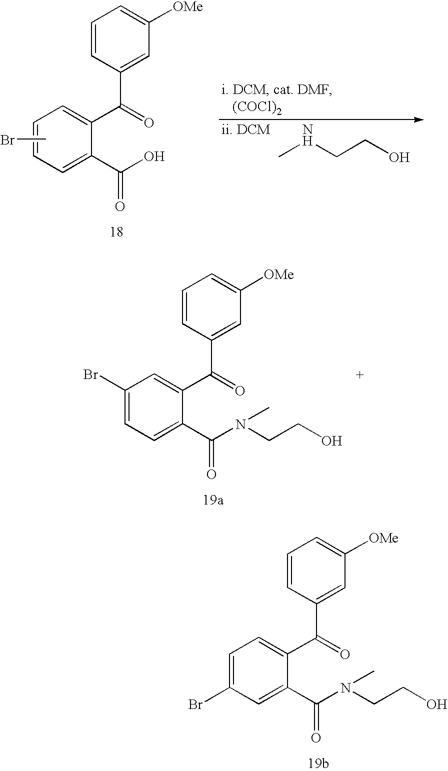

### N-2-hydroxyethyl)-N-methyl-4-bromo-2-[1-hydroxy-1-(3-methoxyphenyl)]methylbenzylamine (20a)

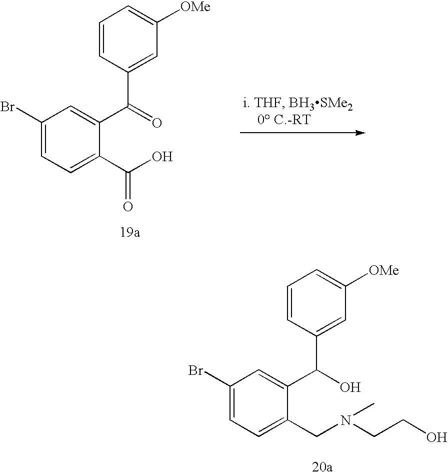

Amide 19a (700 mg, 1.78 mmol) was dissolved in tetrahydrofuran (3 mL) and cooled to 0° C. in an ice bath. A 2M solution of borane dimethylsulfide complex (3.6 mL, 7.12 mmol, 4.4 equiv.) was added dropwise and the resulting solution stirred at room temperature for 17 hours. The reaction was carefully quenched with 1 M hydrochloric acid solution and the resulting solution stirred overnight. Tetrahydrofuran was removed under reduced pressure and the remaining solution washed with diethyl ether until all by-products had been removed. The aqueous layer was basified with NaOH (2M) and extracted using ethyl acetate (30 ml). After washing with NaOH (2×50 ml) and water (50 ml), the ethyl acetate was dried over MgSO₄, filtered and concentrated under reduced pressure to furnish the desired product 20a as a viscous oil. Yield 400 mg, 59%.

¹H nmr (250 MHz, CDCl₃); 7.37-7.31 (2H, m, CHₐᵣ), 7.21 (1H, t, J 8.5, CHₐᵣ), 7.05-6.98 (2H, m, CHₐᵣ), 6.85-6.77 (2H, m, CHₐᵣ), 5.75 (1H, S, CHOH), 3.77 (3H, s, OCH₃), 3.65-3.61 (2H, m, OCH₂), 3.29 (2H, dd, J, 25.3, 13.5, ArCHₐH_{b}N), 2.50 (2H, dd, J 10.9, 5.4, NCH₂), 2.02 (3H, S, NCH₃).

### N-(2-hydroxyethyl)-N-methyl-5-bromo-2-[1-hydroxy-1-(4-methoxyphenyl)]methylbenzylamine (20b)

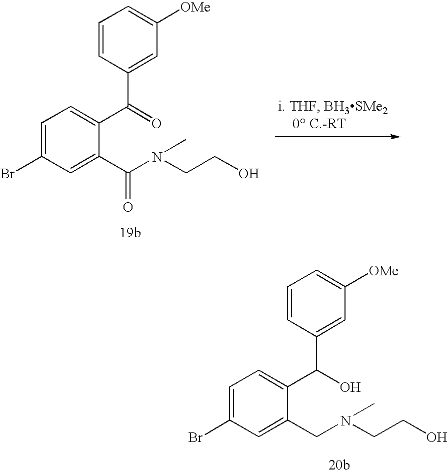

Amide 19b (550 mg, 1.40 mmol) was dissolved in tetrahydrofuran (3 mL) and cooled to 0° C. in an ice bath. A 2M solution of borane dimethylsulfide complex (2.8 mL, 5.60 mmol, 4.4 equiv.) was added dropwise and the resulting solution stirred at room temperature for 17 hours. The reaction was carefully quenched with 1M hydrochloric acid solution and the resulting solution stirred overnight. Tetrahydrofuran was removed under reduced pressure and the remaining solution washed with diethyl ether until all by-products had been removed. The aqueous layer was basified with NaOH (2M) and extracted using ethyl acetate (30 ml). After washing with NaOH (2×50 ml) and water (50 ml), the ethyl acetate was dried over MgSO₄, filtered and concentrated under reduced pressure to furnish the desired product 20b as a viscous oil. Yield 300 mg, 56%.

¹H nmr (250 MHz, CDCl₃); 7.40-7.35 (3H, m, CHₐᵣ), 7.26-7.20 (2H, m, CHₐᵣ), 7.07 (1H, d, J 8.6, CHₐᵣ), 5.80 (1H, S, CHOH), 3.79 (3H, S, OCH₃) 3.71-3.66 (2H, m, OCH₂), 3.36 (2H, dd, J, 19.6, 12.5, ArCH₂N), 2.62-2.49 (2H, m, NCH₂), 2.04 (3H, s, NCH₃).

### EXAMPLE 21

### 9-Bromo-5-methyl-1-(3-methoxy)phenyl-1,3,4,6-tetrahydro-5H-benz[f]-2,5-oxazocine (21a)

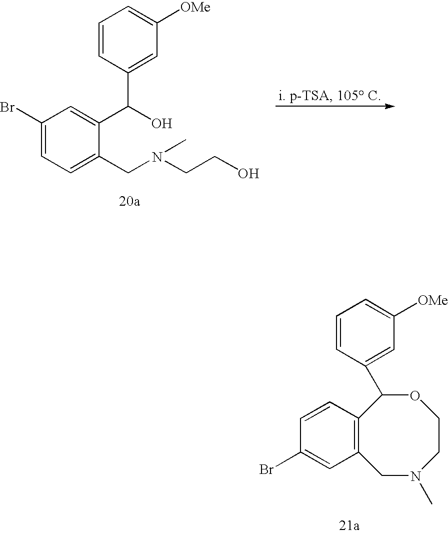

Diol 20a (400 mg, 1.05 mmol) was dissolved in toluene (2 mL) and para-toluenesulfonic acid monohydrate (300 mg, 1.58 mmol, 1.5 equiv.) added. The toluene was removed under reduced pressure and the resulting oil heated at 105° C. for 4 hours. On cooling the oil was suspended in water (100 mL) and basified with 3.75M sodium hydroxide solution. The aqueous layer was extracted with ethyl acetate, dried over magnesium sulfate, filtered and evaporated under reduced pressure to furnish the crude product. Purification by column chorography (10% methanol in ethyl acetate) furnished 21a as yellow solid. Yield 71 mg, 19%.

¹H nmr (250 MHz, CDCl₃); 7.39-7.05 (5H, m, CHₐᵣ), 6.84-6.79 (3H, m, CHₐᵣ), 5.66 (1H, S, CH), 4.78 (1H, d, J 13.8, ArCHₐH_{b}), 4.22-4.13 (1H, m, OCHₐH_{b}), 3.84-3.83 (1H, m, OCHₐH_{b}), 3.77 (3H, s, OCH₃), 3.64 (1H, d, J 13.7, ArCHₐH_{b}), 2.82-2.74 (1H, m NCHₐH_{b}), 2.64-2.57 (1H, m, CHₐH_{b}), 2.42 (3H, s, CH₃).

### EXAMPLE 22

### 8-Bromo-5-methyl-1-(3-methoxy)phenyl-1,3,4,6-tetrahydro-5H-benz[f]-2,5-oxazocine (21b)

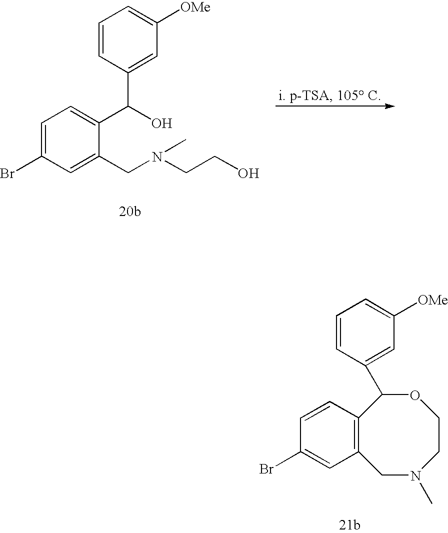

Diol 20b (300 mg, 0.79 mmol) was dissolved in toluene (2 mL) and para-toluenesulfonic acid monohydrate (228 mg, 1.2 mmol, 1.5 equiv.) added. The toluene was removed under reduced pressure and the resulting oil heated at 105° C. for 4 hours. On cooling the oil was dissolved in 3.75M sodium hydroxide solution and extracted with ethyl acetate, dried over magnesium sulfate, filtered and evaporated under reduced pressure to furnish 21b as a brown oil. Yield 75 mg, 26%. No further purification was performed.

¹H nmr (250 MHz, CDCl₃); 7.37-719 (3H, m, CHₐᵣ), 6.89-6.78 (4H, m, CHₐᵣ), 5.70((1H, s, CH), 4.75 (1H, d, J 14, ArCHₐH_{b}), 4.20-4.10 (1H, m, OCHₐH_{b}), 3.86-3.77 (4H, m, ArCHₐH_{b}), OCH₃), 3.60 (1H, d, J 14, ArCHₐH_{b}), 2.80(1H, ddd, J 2.1, 8.5, 15.3, NCHₐH_{b}), 2.62 (1H, ddd, J 2.7, 6.4, 15.3, NCHₐH_{b}), 2.44 (3H, s, CH₃).

### EXAMPLE 23

### 9-Cyano-5-methyl-1-(3-methoxy)phenyl-1,3,4,6-tetrahydro-5H-benz[t]-2,5-oxazocine (22a)

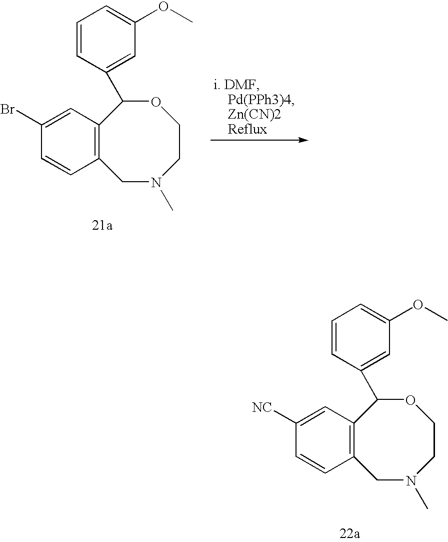

Bromo-nefopam analogue 21a (503 mg, 1.39 mmol), Zn(CN)₂ (245 mg, 2.09 mmol), and Pd(PPh₃)₄ (241 mg, 0.21 mmol), were dissolved in degassed anhydrous DMF (10 mL) under a N₂ atmosphere. The mixture was refluxed under N₂ for 24 hours. The mixture was allowed to cool to room temperature, filtered through celite and washed through with DCM (50 ml). The filtrate was then quenched with water (10 ml) and solvent extracted. The organic extract was dried over MgSO₄, filtered and solvent removed under reduced pressure. The crude product was purified by dry flash chromatography eluting with 5%-15% MeOH in DCM. Fractions containing the product were combined and evaporated under reduced pressure to produce 22a as pale brown oil. Yield 143 mg, 33%.

¹H nmr (250 MHz, CDCl₃); 7.51 (1H, dd, J 1.4, 7.8, CHₐᵣ), 7.32-7.23 (4H, m, CHₐᵣ), 6.84-6.81 (3H, m, CHₐᵣ), 5.76 (1H, s, CHO), 4.85 (1H, d, J 12.8, ArCHₐH_{b}), 4.18-4.15 (1H, m, OCHₐH_{b}), 3.86-3.81 (1H, m, OCHₐH_{b}), 3.79 (3H, s, OCH₃), 3.71 (1H, d, J 12.8, ArCHₐH_{b}),2.78(1H, ddd, J2.3, 8.2, 143, NCHₐH_{b}), 2.63 (1H, ddd, J 2.8, 5.6, 14.3, NCHₐH_{b}), 2.44 (3H, s, NCH₃).

### EXAMPLE 24

### 8-Cyano-5-methyl-1-(3-methoxy)phenyl-1,3,4,6-tetrahydro-5H-benz[f]-2,5-oxazocine (22b)

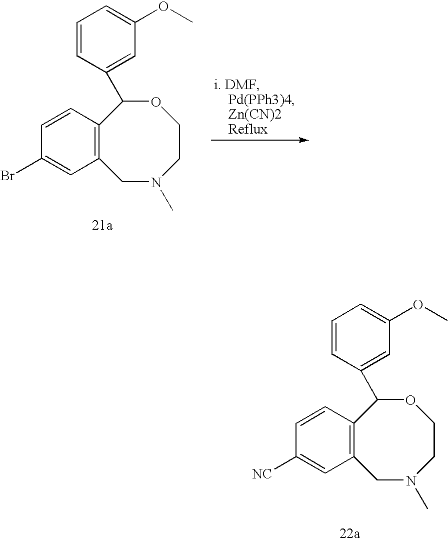

Bromo-nefopam analogue 21b (223 mg, 0.62 mmol), Zn(CN)₂ (109 mg 0.93 mmol), and Pd(PPh₃)₄ (107 mg, 0.09 mmol), were dissolved in degassed anhydrous DMF (5 mL) under a N₂ atmosphere. The mixture was refluxed under N₂ for 24 hours. The mixture was allowed to cool to room temperature, filtered through celite and washed through with DCM (50 ml). The filtrate was then quenched with water (10 ml) and solvent extracted. The organic extract was dried over MgSO₄, filtered and solvent removed under reduced pressure. The crude product was purified by dry flash chromatography eluting with 5%-15% MeOH in DCM. Fractions containing the product were combined and evaporated under reduced pressure to produce 22b as pale brown solid. Yield 94 mg, 50%.

¹H nmr (250 MHz, CDCl₃); 7.50 (1H, s, CHₐᵣ), 7.46 (1H, dd, J 1.7, 8.1, CHₐᵣ), 7.26-7.21 (1H, m, CHₐᵣ), 7.12 (1H, d, J7.9, CHₐᵣ), 6.83-6.80 (3H, m, CHₐᵣ), 5.74 (1H, S, CHO), 4.94 (1H, d, J 13.0, ArCHₐH_{b}), 4.28-4.19 (1H, m, OCHₐH_{b}), 3.87-3.79 (1H, m, OCHₐH_{b}), 3.77 (3H, s, OCH₃), 3.65 (1H, d, J 13.0, ArCHₐH_{b}), 2.75 (1H, ddd, J 2.3, 8.3, 14.3, NCHₐH_{b}), 2.63 (1H, ddd, J 3.0, 5.4, 14.3, NCHₐH_{b}), 2.45 (3H, s, NCH₃).

### EXAMPLE 25

### 9-Methoxy-5-methyl-I-phenyl-1,3,4,6-tetrahydro-5H-benz[f]-2,5-oxazocine (23a)

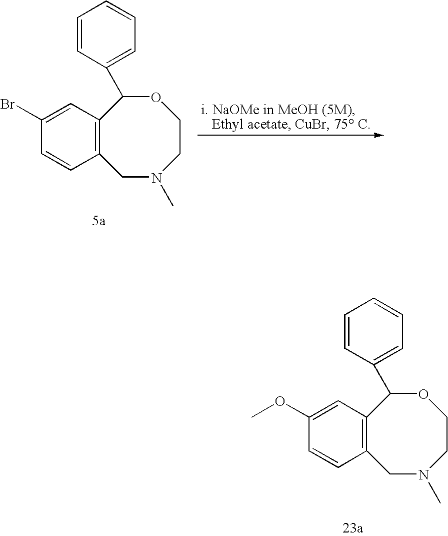

Ethyl acetate (0.1 ml) was added to a stirred 5M NaOMe methanol solution (1 ml) under an N₂ atmosphere at room temperature. Bromo-nefopam analogue 5a (210 mg, 0.58 mmol) in MeOH (1 ml) was then added followed by CuBr (17 mg, 0.12 mmol). The mixture was stirred at 75° C. overnight, cooled to room temperature and quenched using water (5 ml). The organic layer was separated and the aqueous was washed with ethyl acetate (2×10 ml). The combined organics were dried over MgSO₄, filtered and concentrated under reduced pressure. The crude product was purified by dry flash chromatography eluting with 10% MeOH in DCM. Fractions containing the product were combined and evaporated under reduced pressure to produce 23a as an orange oil. Yield 131 mg, 73%.

¹H nmr (250 MHz, CDCl₃); 7.31-7.24 (5H, m, CHₐᵣ), 7.16 (1H, d, J 8.4, CHₐᵣ), 6.77 (1H, dd, J 2.7, 8.3, CHₐᵣ), 6.53 (1H, d, J2.6, CHₐᵣ), 5.72 (1H, s, CHO), 4.74 (1H, d, J 12.8, ArCHₐH_{b}), 4.23-4.15 (1H, m, OCHₐH_{b}), 3.86 (1H, ddd, J2.3, 5.6, 12.6, OCHₐH_{b}), 3.71 (3H, s, OCH₃), 3.68 (1H, d, J 12.8, ArCHₐH_{b}), 2.84 (1H, ddd, J 2.3, 8.5, 14.2, NCHₐH_{b}), 2.62 (1H, ddd, J 2.7, 5.6, 14.3, NCHₐH_{b}), 2.45 (3H, s, NCH₃).

### EXAMPLE 26

### 8-Methoxy-5-methyl-1-phenyl-1,3,4,6-tetrahydro-5H-benz[f]-2,5-oxazocine (23b)

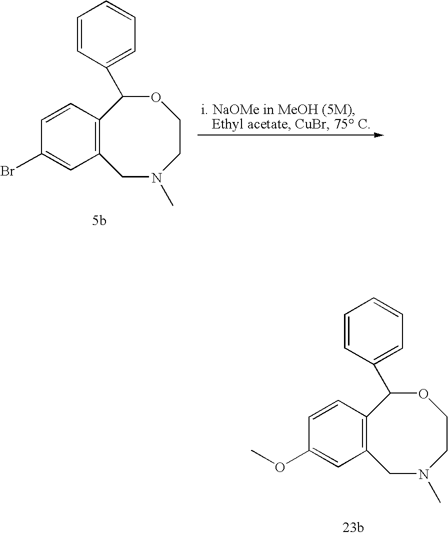

Ethyl acetate (0.1 ml) was added to a stirred 5M NaOMe methanol solution (1 ml) under an N₂ atmosphere at room temperature. Bromo-nefopam analogue 5b (215 mg, 0.60 mmol) in MeOH (1 ml) was then added followed by CuBr (17 mg, 0.12 mmol). The mixture was stirred at 75° C. overnight, cooled to room temperature and quenched using water (5 ml). The organic layer was separated and the aqueous was washed with ethyl acetate (2×10 ml). The combined organics were dried over MgSOₐ, filtered and concentrated under reduced pressure. The crude product was purified by dry flash chromatography eluting with 10% MeOH in DCM. Fractions containing the product were combined and evaporated under reduced pressure to produce 23b as an orange oil. Yield 161 mg, 88%.

¹H nmr (250 MHz, CDCl₃); 7.35-7.20 (5H, m CHₒᵣ), 6.89 (1H, d, J 8.3, CHₐᵣ), 6.76 (1H, s, CHₐᵣ), 6.72 (1H, m, CHₐᵣ), 5.80 (1H, s, CHO), 4.64 (1H, d, J 12.8, ArCHₐH_{b}), 4.11 (1H, ddd, J 2.5, 8.0, 12.7, OCHₐH_{b}), 3.87 (1H, ddd, J 2.2, 6.0, 12.7, OCHₐH_{b}), 3.80 (3H, s, OCH₃), 3.70 (1H, d, J 12.8, ArCHₐH_{b}), 2.87 (1H, ddd, J2.0, 8.0, 14.2, NCHₐH_{b}), 2.66 (1H, ddd, J 2.4, 6.1, 14.2, NCHₐH_{b}),2.48 (3H, S, NCHₑ).

### EXAMPLE 27

### 9-Methoxy-5-methyl-I-(3-methoxy)phenyl-1,3,4,6-tetrahydro-5H-benz[f]-2,5-oxazocine (24a)

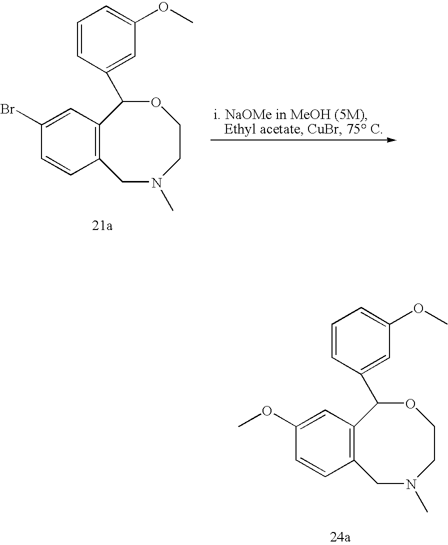

Ethyl acetate (0.1 ml) was added to a stirred 5M NaOMe methanol solution (1 ml) under an N₂ atmosphere at room temperature. Bromo-nefopam analogue 21a (173 mg, 0.48 mmol) in MeOH (1 ml) was then added followed by CuBr (14 mg, 0.10 mmol). The mixture was stirred at 75° C. overnight, cooled to room temperature and quenched using water (5 ml). The organic layer was separated and the aqueous was washed with ethyl acetate (2×10 ml). The combined organics were dried over MgSO₄, filtered and concentrated under reduced pressure. The crude product was purified by dry flash chromatography eluting with 10% MeOH in DCM. Fractions containing the product were combined and evaporated under reduced pressure to produce 24a as an orange oil. Yield 34 mg, 22%.

¹H nmr (250 MHz, CDCl₃); 7.20 (1H, d, J 8.2, CHₐᵣ), 7.15 (1H, s, CHₐᵣ), 6.87-6.84 (3H, m, CHₐᵣ), 6.77 (1H, dd, J2.6, 8.3, CHₐᵣ), 6.54 (1H, d, J2.6, CHₐᵣ), 5.68 (1H, s, CHO), 4.75 (1H, d, J 12.8, ArCHₐH_{b}), 4.24-4.15 (1H, m, OCHₐH_{b}), 3.85(1H, ddd, J 2.4, 5.3, 12.7, OCHₐH_{b}), 3.76 (3H, s, OCH₃), 3.71(1H, d, J 12.8, ArCHₐH_{b}), 3.70 (3H, s, OCH₃), 2.85 (1H, ddd, J2.1, 8.7, 14.1, NCHₐH_{b}), 2.64 (1H, ddd, J2.7, 5.3, 14.3, NCHₐH_{b}), 2.47 (3H, s, NCH₃).

### EXAMPLE 28

### 8-Methoxy-5-methyl-1-(3-methoxy)phenyl-1,3,4,6-tetrahydro-5H-benz[fJ-2,5-oxazocine (24b)

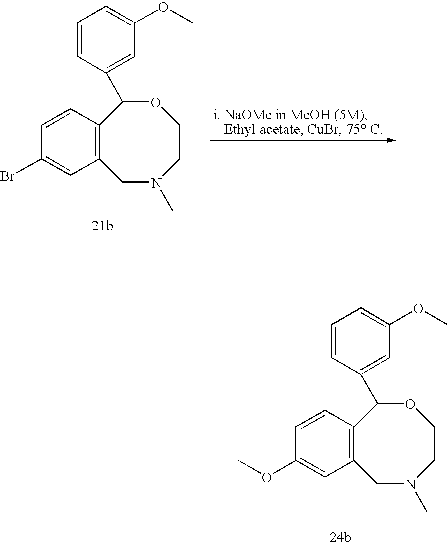

Ethyl acetate (0.1 ml) was added to a stirred 5M NaOMe methanol solution (1 ml) under an N₂ atmosphere at room temperature. Bromo-nefopam analogue 21b (168 mg, 0.47 mmol) in MeOH (1 ml) was then added followed by CuBr (13 mg, 0.09 mmol). The mixture was stirred at 75° C. overnight, cooled to room temperature and quenched using water (5 ml). The organic layer was separated and the aqueous was washed with ethyl acetate (2×10 ml). The combined organics were dried over MgSO₄, filtered and concentrated under reduced pressure. The crude product was purified by dry flash chromatography eluting with 10% MeOH in DCM. Fractions containing the product were combined and evaporated under reduced pressure to produce 24b as an orange oil. Yield 77 mg, 50%.

¹H nmr (250 MHz, CDCl₃); 7.22 (1H, t, J 8.2, CHₐᵣ), 6.92-6.71 (6H, m, CHₐᵣ), 5.78 (1H, s, CHO), 4.60 (1H, d, J 12.7, ArCHₐH_{b}), 4.09 (1H, ddd, J 2.4, 7.9, 12.7, OCHₐH_{b}), 3.90-3.82 (1H, m, OCHₐH_{b}), 3.80 (3H, S, OCH₃), 3.77 (3H, S, OCH3), 3.70 (1H, d, J 12.8, ArCHₐH_{b}), 2.85 (1H, ddd, J 1.9, 7.9, 14.2, NCHₐH_{b}), 2.65 (1H. ddd, J2.5, 6.1, 14.2, NCHₐH_{b}), 2.47 (3H, s, NCH₃).

### Cyclopropylboronic Acid (25)

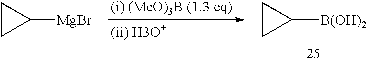

To a stirred solution of trimethylborate (1.69 g, 1.81 ml, 16.25 mmol) in THF (7 ml) at −78° C. under a N₂ atmosphere was added, by drop wise addition, cyclopropylmagnesium bromide (0.5M in THF, 25 m1, 12.5 mmol). A white precipitate formed. After 1 hr the reaction was warmed to room temperature and stirred overnight. The reaction was quenched with HCl aq. (20 ml, 2. ON) and the mixture stirred for 1 hour. After extracting with DCM (15 ml) and back extracting with H₂O (2×15 ml), the aqueous fractions were combined and extracted using TBME (4×40 ml). The combined organic extracts were dried over MgSO₄ and concentrated under reduced pressure to give a white solid. Recrystallisation from DCM and hexane (twice) furnished a white solid 25, 297 mg, 27% yield.

¹H nmr (250 MHz, CDCl₃); 0.56-0.50 (2H, m, CH₂), 0.42-0.40 (2H, m, CH₂), −0.08-0.20 (1H, m, CH).

### EXAMPLE 29

### 9-Cyclopropyl-5-methyl-1-phenyl-1,3,4,6-tetrahydro-SH-benz[f]-2,5-oxazocine (26a)

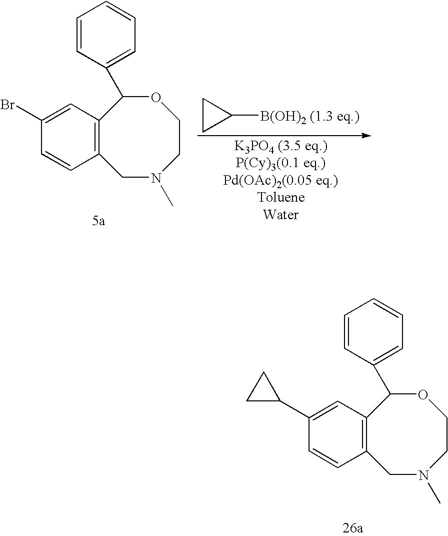

To a stirred solution of bromo-nefopam analogue 5a (211 mg, 0.64 mmol), cyclopropyl boronic acid (71 mg, 0.83 mmol), potassium phosphate (472 mg, 2.22 mmol) and tricyclohexylphosphine (18 mg, 0.06 mmol) in toluene (5 ml) and water (250 μl) under a N₂ atmosphere was added palladium acetate (7 mg, 0.03 mmol). The mixture was heated to 100° C. for 3hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 15% MeOH in DCM. Fractions containing the product were combined and evaporated under reduced pressure to produce 26a as a yellow oil. Yield 145 mg, 78%.

¹H nmr (250 MHz, CDCl₃); 7.32-7.24 (5H, m, CHₐᵣ), 7.11 (1H, d, J 7.8, CHₐᵣ), 6.87 (1H, dd, J 1.8, 7.8, CHₐᵣ), 6.73 (1H, d, J 1.5, CHₐᵣ), 5.74 (1H, S, CHO), 4.75 (1H, d, J 12.8, ArCHₐH_{b}), 4.17 (1H, ddd, J2.8, 8.3, 12.4, OCHₐH_{b}), 3.85 (1H, ddd, J 2.3, 6.0, 12.5, OCHₐH_{b}), 3.63 (1H, d, J 12.8, ArCHₐH_{b}), 2.81 (1H, ddd, J 2.2, 8.2, 14.1, NCHₐH_{b}), 2.61 (1H, ddd, J2.7, 5.9, 14.2, NCHₐH_{b}), 2.44 (3H, s, NCH₃), 1.77 (1H, m, CH), 0.91-0.86 (2H, m, CH₂), 0.61-0.57 (2H, m, CH₂)

### EXAMPLE 30

### 8-Cyclopropyl-5-methyl-1-phenyl-1,3,4,6-tetrahydro-5H-benz[f]-2,5-oxazocine (26b)

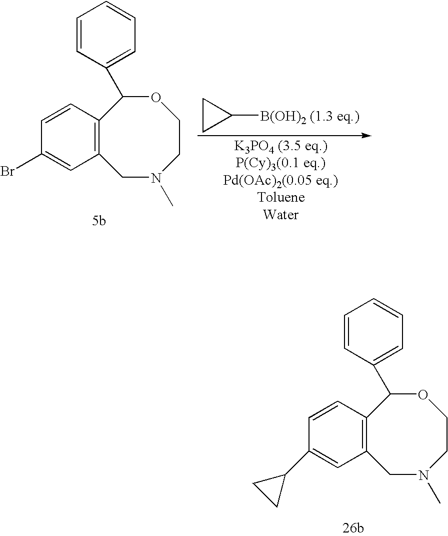

To a stirred solution of bromo-nefopam analogue 5b (178 mg, 0.54 mmol), cyclopropyl boronic acid (60 mg, 0.70 mmol), potassium phosphate (398 mg, 1.88 mmol) and tricyclohexylphosphine (15 mg, 0.05 mmol) in toluene (4 ml) and water (200 μl) under a N₂ atmosphere was added palladium acetate (6 mg, 0.03 mmol). The mixture was heated to 100° C. for 3 hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 15% MeOH in DCM. Fractions containing the product were combined and evaporated under reduced pressure to produce 26b as a yellow oil. Yield 132 mg, 84%.

¹H nmr (250 MHz, CDCl₃); 7.31-7.23 (5H, m, CHₐᵣ), 6.94 (1H, s, CHₐᵣ), 6.86 (2H, d, J 1.0, CHₐᵣ), 5.78 (1H, s, CHO), 4.70 (1H, d, J 12.5, ArCHₐH_{b}), 4.15 (1H, ddd, J 2.6, 8.2, 12.6, OCHₐH_{b}), 3.86 (1H, ddd, J 2.1, 6.0, 12.7, OCHₐH_{b}), 3.66 (1H, d, J12.7, ArCHₐH_{b}), 2.85 (1H, ddd, J2.1, 8.2, 14.2, NCHₐH_{b}), 2.63 (1H, ddd,J2.6, 6.0, 14.2, NCHₐH_{b}), 2.48 (3H, s, NCH₃), 1.86 (1H, m, CH), 0.96-0.90 (2H, m, CH₂), 0.72-0.66 (2H, m, CH₂).

### EXAMPLE 31

### N-(Acetyl)-5-methyl-1-phenyl-1,3,4,6-tetrahydro-5H-benz[t]-2,5-oxazocine-9-methylamine (27a)

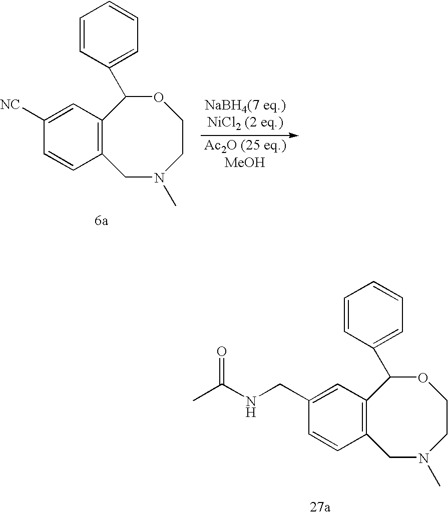

NaBH₄ (129 mg, 3.41 mmol) was cautiously added to a solution of NiCl₂ (126 mg, 0.97 mmol), acetic anhydride (0.1 ml, 0.97 mmol) and bromo-nefopam analogue 6a (134 mg, 0.49 mmol) in MeOH (8 ml) at room temperature under a N₂ atmosphere. Once the vigorous reaction had subsided the mixture was left to stir at room temperature overnight. Methanol was removed under reduced pressure and the precipitate partially dissolved in EtOAc and NaHCO₃ (aq.). After filtration the green solid was repeatedly washed with EtOAc and NaHCO₃ (aq.). The organic filtrate was separated from the aqueous which was subsequently extracted with more EtOAc (3×10 ml). The combined organic fractions were dried over MgSO₄, filtered and concentrated under reduced pressure. The crude product was purified by dry flash chromatography eluting with 20% MeOH in DCM. Fractions containing the product were combined and evaporated under reduced pressure to produce 27a as an orange oil. Yield 36 mg, 23%.

¹H nmr (250 MHz, CDCl₃); 7.33-7.10 (7H, m, CHₐᵣ), 6.87 (1H, s, CHₐᵣ), 5.91 (1H, brs, NH), 5.73 (1H, s, CHO), 4.80 (1H, d, J 12.7, ArCHₐH_{b}), 4.29 (1H, d, J 2.9, NHCHₐCH_{b}), 4.27 (1H, d, J 2.9, NHCHₐCH_{b}), 4.17 (1H, m, OCHₐH_{b}), 3.82 (1H, ddd, J 2.1, 5.6, 12.6, OCHₐH_{b}), 3.65 (1H, d, J 12.7, ArCHₐH_{b}), 2.78 (1H, ddd, J2.1, 8.4, 14.1, NCHₐH_{b}), 2.59 (1H, ddd, J2.5, 5.6, 14.1, NCHₐH_{b}), 2.43 (3H, s, NCH₃), 1.93 (3H, s, CH₃CO).

### EXAMPLE 32

### N-(Acetyl)-5-methyl-1-phenyl-1,3,4,6-tetrahydro-SH-benz[f]-2,5-oxazocine-8-methylamine (27b)

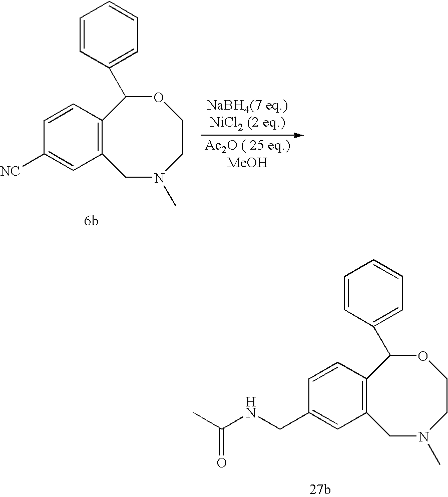

NaBH₄ (148 mg, 3.92 mmol) was cautiously added to a solution of NiC1₂ (145 mg, 1.12 mmol), acetic anhydride (0.11 ml, 1.12 mmol) and bromo-nefopam analogue 6b (154 mg, 0.56 mmol) in MeOH (8 ml) at room temperature under a N₂ atmosphere. Once the vigorous reaction had subsided the mixture was left to stir at room temperature overnight. Methanol was removed under reduced pressure and the precipitate partially dissolved in EtOAc and NaHCO₃ (aq.). After filtration the green solid was repeatedly washed with EtOAc and NaHCO₃ (aq.). The organic filtrate was separated from the aqueous which was subsequently extracted with more EtOAc (3×10 ml). The combined organic fractions were dried over MgSO₄, filtered and concentrated under reduced pressure. The crude product was purified by dry flash chromatography eluting with 10-15% MeOH in DCM. Fractions containing the product were combined and evaporated under reduced pressure to produce 27b as an orange oil. Yield 83 mg, 46%.

¹H nmr (250 MHz, CDCl₃); 7.30-7.23 (5H, m, CHₐᵣ), 7.14 (1H, s, CHₐᵣ), 7.11 (1H, d, J 8.1, CHₐᵣ), 6.97 (1H, d, J 7.8, CHₐᵣ), 5.83 (1H, brs, NH), 5.77 (1H, s, CHO), 4.75 (1H, d, J 12.5, ArCHₐH_{b}), 4.39 (2H, d, J 5.7, NHCH₂), 4.21-4.16 (1H, m, OCHₐH_{b}), 3.83 (1H, ddd, J 2.1, 6.2, 12.6, OCHₐH_{b}), 3.64 (1H, d, J 12.7, ArCHₐH_{b}), 2.84 (1H, ddd, J 2.1, 7.9, 14.2, NCHₐH_{b}), 2.62 (1H, ddd, J 2.6, 6.1, 14.2, NCHₐH_{b}), 2.49 (3H, S, NCH₃), 2.00 (3H, S, CH₃CO).

### EXAMPLE 33

### N-(Methylsulphonyl)-5-methyl-1-phenyl-1,3,4,6-tetrahydro-SH-benz[f]-2,5-oxazocine-9-methylamine (28b)

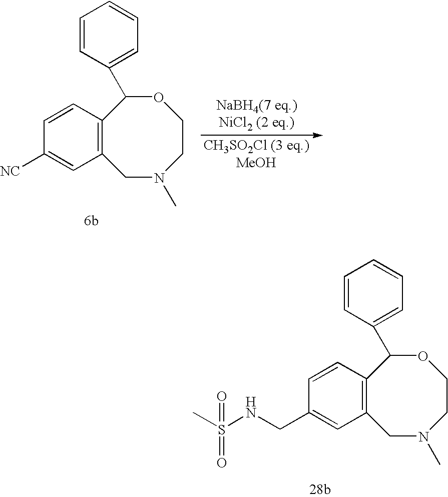

NaBH₄ (193 mg, 5.11 mmol) was cautiously added to a solution of NiCl₂ (188 mg, 1.45 mmol), methanesulfonyl chloride (0.17 ml, 2.19 mmol) and bromo-nefopam analogue 6b (200 mg, 0.73 mmol) in MeOH (8 ml) at room temperature under a N₂ atmosphere. Once the vigorous reaction had subsided the mixture was left to stir at room temperature overnight. Methanol was removed under reduced pressure and the precipitate partially dissolved in EtOAc and NaHCO₃ (aq.). After filtration the green solid was repeatedly washed with EtOAc and NaHCO₃ (aq.). The organic filtrate was separated from the aqueous which was subsequently extracted with more EtOAc (3×10 ml). The combined organic fractions were dried over MgSO4, filtered and concentrated under reduced pressure. The crude product was purified by dry flash chromatography eluting with 15-20% MeOH in DCM. Fractions containing the product were combined and evaporated under reduced pressure to produce 28b as a white solid. Yield 54 mg, 21%.

¹H nmr (250 MHz, CDCl₃); 7.95 (1H, s, CHₐᵣ), 7.77 (1H, dd, J 1.5, 8.1, CHₐᵣ), 7.39 (1H, d, J 8.2, CHₐᵣ), 7.33-7.24 (5H, m, CHₐᵣ), 6.00 (1H, s, CHO), 5.99 (1H, d, J 12.7, ArCHₐH_{b}), 4.64 (1H, m, OCHₐH_{b}), 4.48 (1H, d, J 12.8, ArCHₐH_{b}), 4.15 (1H, m, OCHₐH_{b}), 3.47 (3H, s, CH₃SO₂), 3.44 (1H, m, NCHₐH_{b}), 3.31 (1H, m, NCHₐH_{b}), 3.08 (2H, s, NHCH₂), 2.70 (3H, s, NCH₃).

### EXAMPLE 34

### 9-Cyclopropyl-5-methyl-1-(3-methoxy)phenyl-1,3,4,6-tetrahydro-5H-benz[f]-2,5-oxazocine (29a)

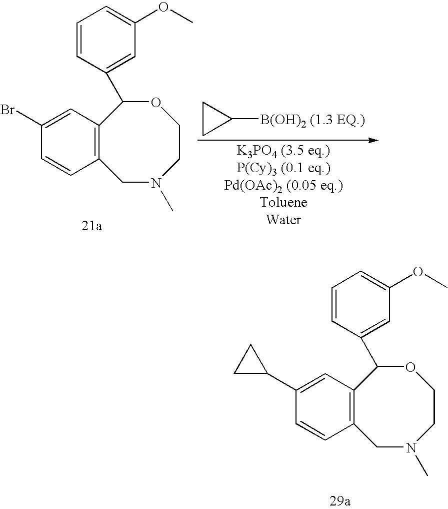

To a stirred solution of bromo-nefopam analogue 21a (104 mg, 0.29 mmol), cyclopropyl boronic acid (32 mg, 0.37 mmol), potassium phosphate (214 mg, 1.Ommol) and tricyclohexylphosphine (8 mg, 0.03 mmol) in toluene (5 ml) and water (250 μl) under a N₂ atmosphere was added palladium acetate (3 mg, 0.01 mmol). The mixture was heated to 100° C. for 3 hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 5% MeOH in DCM. Fractions containing the product were combined and evaporated under reduced pressure to produce 29a as an orange oil. Yield 65 mg, 70%.

¹H nmr (250 MHz, CDCl₃); 7.23 (1H, t, J 8.1, CHₐᵣ), 7.10 (1H, d, J 7.8, CHₐᵣ), 6.88-6.75 (5H, m, CHₐᵣ), 5.70 (1H, s, CHO), 4.75 (1H, d, J 12.7, ArCHₐH_{b}), 4.21-4.12 (1H, m, OCHₐH_{b}), 3.84 (1H, ddd, J 2.3, 5.7, 12.6, OCHₐH_{b}), 3.77 (3H, S, OCH₃), 3.65 (1H, d, J 12.7, ArCHₐH_{b}), 2.82 (1H, ddd, J 2.2, 8.3, 14.1, NCHₐH_{b}), 2.62 (1H, ddd, J2.7, 5.7, 14.2, NCHₐH_{b}), 2.45 (3H, S, NCH₃), 1.77 (1H, m, CH), 0.91-0.87 (2H, m, CH₂), 0.62-0.57 (2H, m, CH₂).

### EXAMPLE 35

### 8-Cyclopropyl-5-methyl-1-(3-methoxy)phenyl-1,3,4,6-tetrahydro-5H-benz[f]

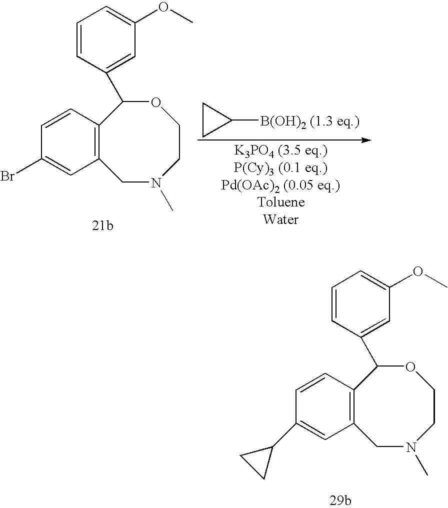

To a stirred solution of bromo-nefopam analogue 21b (115 mg, 0.32 mmol), cyclopropyl boronic acid (36 mg, 0.42 mmol), potassium phosphate (241 mg, 1.13 mol) and tricyclohexylphosphine (9 mg, 0.03 mmol) in toluene (4 ml) and water (250 μl) under a N₂ atmosphere was added palladium acetate (4 mg, 0.02 mmol). The mixture was heated to 100° C. for 3 hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 5% MeOH in DCM. Fractions containing the product were combined and evaporated under reduced pressure to produce 29b as an orange oil. Yield 77 mg, 75%.

¹H nmr (250 MHz, CDCl₃); 7.22 (1H, t, J 8.1, CHₐᵣ), 6.94-6.76 (6H, m, CHₐᵣ), 5.74 (1H, s, CHO), 4.69 (1H, d, J 12.7, ArCHₐH_{b}), 4.14 (1H, ddd, J 2.6, 8.4, 12.6, OCHₐH_{b}), 3.85 (1H, ddd, J2.1, 5.8, 12.7, OCHₐH_{b}), 3.77 (3H, s, OCH_{'}), 3.68 (1H, d, J 12.7, ArCHₐH_{b}), 2.85 (1H, ddd, J 2.0, 8.3, 14.2, NCHₐH_{b}), 2.64 (1H, ddd, J 2.5, 5.8, 14.3, NCHₐH_{b}), 2.49 (3H, s, NCH₃), 1.86 (1H, m, CH), 0.93 (2H, m, CH₂), 0.68 (2H, m, CH₂).

### EXAMPLE 36

### 9-Cyclopropyl-5-methyl-1-(4-methoxy)phenyl-1,3,4,6-tetrahydro-5H-benz[f]-2,5-oxazocine (30a)

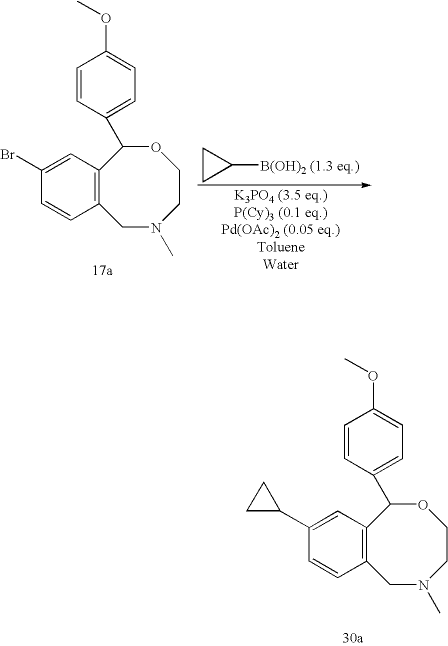

To a stirred solution of bromo-nefopam analogue 17a (110 mg, 0.31 mmol), cyclopropyl boronic acid (34 mg, 0.40 mmol), potassium phosphate (226 mg, 1.07 mmol) and tricyclohexylphosphine (9 mg, 0.03 mmol) in toluene (4 ml) and water (200 μl) under a N₂ atmosphere was added palladium acetate (4 mg, 0.02 mmol). The mixture was heated to 100° C. for 3 hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 5% MeOH in DCM. Fractions containing the product were combined and evaporated under reduced pressure to produce 30a as an orange oil. Yield 70 mg, 71%.

¹H nmr (250 MHz, CDCl₃); 7.18 (2H, d, J 8.5, CHₐᵣ), 7.12 (1H, d, J 7.8, CHₐᵣ), 6.88 (1H, d, J 1.4, CHₐᵣ), 6.84 (2H, d, J 8.7, CHₐᵣ) 6.73 (1H, d, J 1.2, CHₐᵣ), 5.68 (1H, s, CHO), 4.80 (1H, d, J 12.7, ArCHₐH_{b}), 4.18 (1H, m, OCHₐH_{b}), 3.83 (1H, m, OCHaH_{b}), 3.77 (3H, s, OCH₃), 3.64 (1H, d, J 12.7, ArCHₐH_{b}), 2.81 (1H, ddd, J 2.2, 8.5, 14.0, NCHₐH_{b}), 2.60 (1H, ddd, J 2.8, 5.5, 14.0, NCHₐH_{b}), 2.46 (3H, s, NCH₃), 1.77 (1H, m, CH), 0.87 (2H, m, CH₂), 0.60 (2H, m, CH₂).

### EXAMPLE 37

### 8-Cyclopropyl-5-methyl-1-(4methoxy)phenyl-1,3,4,6-tetrahydro-5H-benz[f]-2,5-oxazocine (30b)

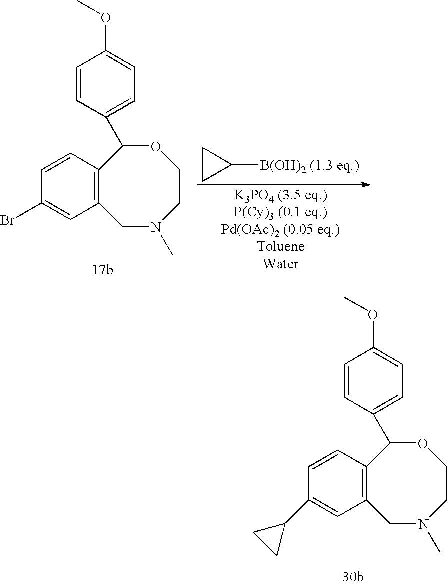

To a stirred solution of bromo-nefopam analogue 17b (117 mg, 0.32 mmol), cyclopropyl boronic acid (36 mg, 0.42 mmol), potassium phosphate (241 mg, 1.13 mmol) and tricyclohexylphosphine (9 mg, 0.03 mmol) in toluene (4 ml) and water (200 μl) under a N₂ atmosphere was added palladium acetate (4 mg, 0.02 mmol). The mixture was heated to 100° C. for 3 hrs and then cooled to room temperature. Water (10 ml) was added and the mixture extracted with ethyl acetate (2×15 ml). The combined organic extracts were washed with brine (10 ml), dried over MgSO₄ and concentrated under reduced pressure to give a yellow oil. The crude product was purified by dry flash chromatography eluting with 5% MeOH in DCM. Fractions containing the product were combined and evaporated under reduced pressure to produce 30b as an orange oil. Yield 62 mg, 60%.

¹H nmr (250 MHz, CDCl₃); 7.17 (2H, d, J 8.5, CHₐᵣ), 6.95 (1H, S, CHₐᵣ), 6.84 (4H, m, CHₐᵣ), 5.71 (1H, s, CHO), 4.77 (1H, d, J 12.5, ArCHₐH_{b}), 4.17 (1H, m, OCHₐH_{b}), 3.84 (1H, ddd, J 2.3, 5.5, 12.6, OCHₐH_{b}), 3.76 (3H, S, OCH₃), 3.66 (1H, d, J 12.5, ArCHₐH_{b}), 2.84 (1H, ddd, J2.1, 8.5, 14.0, NCHₐH_{b}), 2.61 (1H, ddd, J 2.7, 5.5, 14.1, NCHₐH_{b}), 2.49 (3H, s, NCH₃), 1.85 (1H, m, CH), 0.93 (2H, m, CH₂), 0.67 (2H, m, CH₂).

### EXAMPLE 38

### 9-Methoxy-5-methyl-1-(4-methoxy)phenyl-1,3,4,6-tetrahydro-5H-benz[f]-2,5-oxazocine (31a)

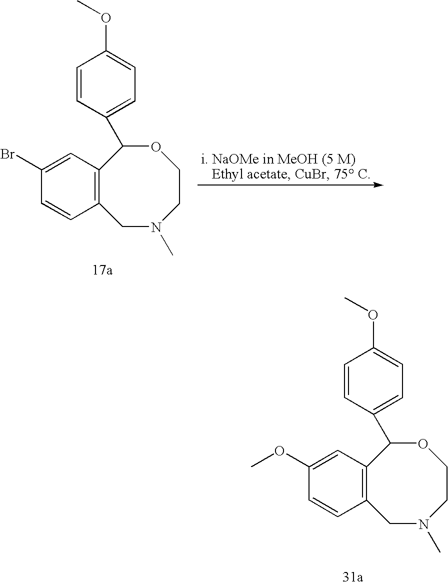

Ethyl acetate (0.1 ml) was added to a stirred 5M NaOMe methanol solution (1 ml) under an N₂ atmosphere at room temperature. Bromo-nefopam analogue 17a (104 mg, 0.29 mmol) in MeOH (1 ml) was then added followed by CuBr (8 mg, 0.06 mmol). The mixture was stirred at 75° C. overnight, cooled to room temperature and quenched using water (5 ml). The organic layer was separated and the aqueous was washed with ethyl acetate (2×10 ml). The combined organics were dried over MgSO₄, filtered and concentrated under reduced pressure. The crude product was purified by dry flash chromatography eluting with 10% MeOH in DCM. Fractions containing the product were combined and evaporated under reduced pressure to produce 31a as an orange oil. Yield 67 mg, 71%.

¹H nmr (250 MHz, CDCl₃); 7.18 (2H, d, J 8.5, CHₐᵣ), 7.19(1H, s, CHₐᵣ), 6.83 (2H, d, J8.6, CHₐᵣ), 6.77 (1H, dd, J2.5, 8.5, CHₐᵣ), 6.52 (1H, d, J2.3, CHₐᵣ), 5.72 (1H, s, CHO), 4.80 (1H, d, J 12.8, ArCHₐH_{b}), 4.20 (1H, m, OCHₐH_{b}), 3.84 (1H, ddd, J2.4, 5.1, 12.8, OCHₐH_{b}), 3.77 (3H, s, OCH₃), 3.71 (3H, s, OCH₃), 3.68 (1H, d, J 12.8, ArCHₐH_{b}), 2.85 (1H, m, NCHₐH_{b}), 2.61 (1H, m, NCHₐH_{b}), 2.47 (3H, s, NCH₃).

### EXAMPLE 39

### 8-Methoxy-5-methyl-1-(4-methoxy)phenyl-1,3,4,6-tetrahydro-5H-benz[fi]-2,5-oxazocine (31b)

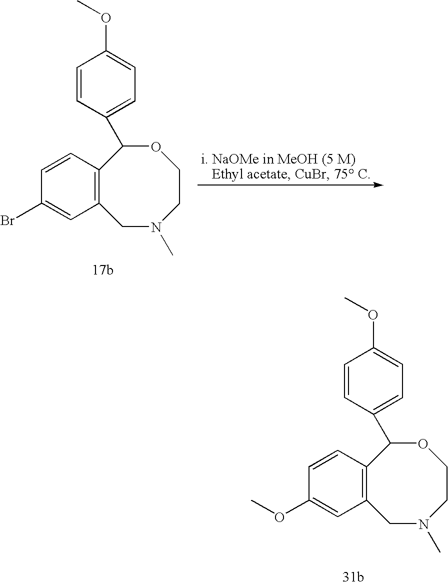

Ethyl acetate (0.1 ml) was added to a stirred 5M NaOMe methanol solution (1 ml) under an N₂ atmosphere at room temperature. Bromo-nefopam analogue 17b (115 mg, 0.32 mmol) in MeOH (1 ml) was then added followed by CuBr (9 mg, 0.06 mmol). The mixture was stirred at 75° C. overnight, cooled to room temperature and quenched using water (5 ml). The organic layer was separated and the aqueous was washed with ethyl acetate (2×10 ml). The combined organics were dried over MgSO₄, filtered and concentrated under reduced pressure. The crude product was purified by dry flash chromatography eluting with 10% MeOH in DCM. Fractions containing the product were combined and evaporated under reduced pressure to produce 31b as an orange oil. Yield 39 mg, 37%.

¹H nmr (250 MHz, CDCl₃); 7.18 (2H, d, J 8.6, CHₐᵣ), 6.91 (1H, s, CHₐᵣ), 6.84 (2H, d, J 8.5, CHₐᵣ), 6.76 (1H, S, CHₐᵣ), 6.73 (1H, d, J 2.6, CHₐᵣ), 5.73 (1H, S, CHO), 4.71 (1H, d, J 12.7, ArCHₐH_{b}), 4.13 (1H, m, OCHₐH_{b}), 3.86 (1H, m, OCHₐH_{b}), 3.80 (3H, S, OCH₃), 3.77 (3H, S, OCH₃), 3.68 (1H, d, J 12.7, ArCHₐH_{b}), 2.86 (1H, m, NCHₐH_{b}), 2.64 (1H, ddd, J 2.5, 5.7, 14.1 NCHₐH_{b}), 2.48 (3H, s, NCH₃).

### EXAMPLE 40

### 9-Cyano-5-methyl-1-(4-methoxy)phenyl-1,3,4,6-tetrahydro-5H-benz[f]-2,5-oxazocine (32a)

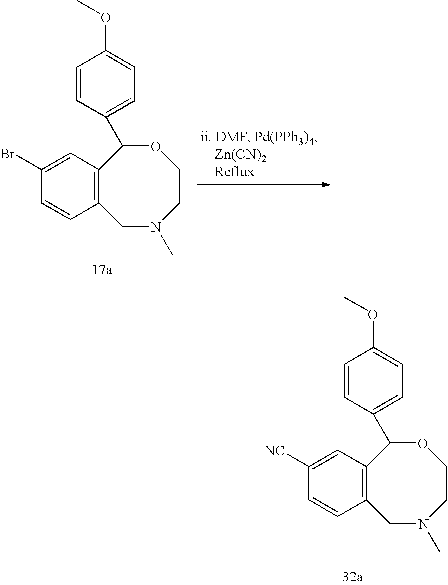

Bromo-nefopam analogue 17a (133 mg, 0.37 mmol), Zn(CN)₂ (65 mg, 0.55 mmol), and Pd(PPh₃)₄ (63 mg, 0.06 mmol), were dissolved in degassed anhydrous DMF (4 mL) under a N₂ atmosphere. The mixture was refluxed under N₂ for 24 hours. The mixture was allowed to cool to room temperature, filtered through celite and washed through with DCM (50 ml). The filtrate was then quenched with water (10 ml) and solvent extracted. The organic extract was dried over MgSO₄, filtered and solvent removed under reduced pressure. The crude product was purified by dry flash chromatography eluting with 5%-15% MeOH in DCM. Fractions containing the product were combined and evaporated under reduced pressure to produce 32a as pale brown oil. Yield 32 mg, 26%.

¹H nmr (250 MHz, CDCl₃); 7.50 (1H, d, J 7.6, CHₐᵣ), 7.31 (2H, d, J 9.0, CHₐᵣ), 7.15 (2H, d, J 8.2, CHₐᵣ), 6.86 (2H, d, J 8.2, CHₐᵣ), 5.73 (1H, s, CHO), 4.91 (1H, d, J 12.8, ArCHₐH_{b}), 4.20 (1H, m, OCHₐH_{b}), 3.82 (4H, brm, OCHₐH_{b}, OCH₃), 3.68 (1H, d, J 12.8, ArCHₐH_{b}),2.76 (1H, m, NCHₐH_{b}), 2.59 (1H, m, NCHₐH_{b}), 2.44 (3H, s, NCH₃).

The following Examples include compounds which are in the following Table with reference to formula 1A).

| Example | R1 | R2 | W, X, Y, Z |
|---|---|---|---|
| 41 | benzyl | phenyl | W═X═Y═Z═CH |
| 53 | methyl | (3-tosyloxy)phenyl | W═X═Z═CH |
| | | | Y═C—Br |
| 60 | methyl | (2-methyl-3- | W═X═Z═CH |
| | | methoxy)phenyl | Y═C—Br |
| 61 | methyl | (2-methyl-3- | W═X═Z═CH |
| | | methoxy)phenyl | Y═C—CN |
| 62 | methyl | (2-methyl-3- | W═X═Z═CH |
| | | methoxy)phenyl | Y═C-cyclopropyl |
| 84 | methyl | 2-methylphenyl | W═Y═Z═CH |
| | | | X═C—Br |
| 85 | methyl | 2-methylphenyl | W═Y═Z═CH |
| | | | X═C—CN |
| 86 | methyl | 2-methylphenyl | W═Y═Z═CH |
| | | | X═C-cyclopropyl |
| 87 | methyl | 2-methylphenyl | W═X═Z═CH |
| | | | Y═C—Br |
| 88 | methyl | 2-methylphenyl | W═X═Z═CH |
| | | | Y═C—CN |
| 89 | methyl | 2-methylphenyl | W═X═Z═CH |
| | | | Y═C-cyclopropyl |
| 90 | methyl | 3-pyridyl | W═Y═Z═CH |
| | | | X═C—Br |
| 91 | methyl | 3-pyridyl | W═X═Z═CH |
| | | | Y═C—Br |
| 92 | methyl | 3-pyridyl | W═X═Z═CH |
| | | | Y═C—CN |

All ¹H NMR recorded on a Bruker AC250. All chemical shifts (δ) have been rounded to 2 decimal places, and coupling constants (J) are measured in Hz and to the nearest 0.1 decimal place.

### EXAMPLE 41

### 5-Benzyl-1-phenyl-1,3,4,6-tetrahydro-5H-benz[f]-2,5-oxazocine

To a stirring solution of desmethylnefopam (96 mg, 0.41 mmol) in DMF (3 ml) were added sodium hydride (15 mg, 0.49 mmol) and then benzylbromide (0.7 ml, 0.61 mmol). The resulting blue solution was heated at 65° C. for 2 hours. The reaction was quenched by the addition of water and the organics were extracted into dichloromethane, dried (MgSO₄), filtered and concentrated in vacuo to provide the crude material. Purification by silica gel column chromatography (1→2% MeOH/DCM) furnished the target product (45 mg, 34%) as a brown oil.

δ_{H} (CDCl₃; 250 MHz) 2.74-2.80 (1H, m, one of CH₂), 2.96-3.00 (1H, m, one of CH₂), 3.88-4.07 (4H, m, four of CH₂), 4.27-4.36 (1H, m, one of CH₂), 4.92 (1H, d, J12.7, one of ArCH₂NR₂), 5.82 (1H, s, CHOR), 7.05-7.07 (1H, m, aromatic H), 7.26-7.42 (11H, aromatic H, 7.56-7.60 (2H, m, aromatic H)

### EXAMPLE 42

### 5-Allyl-1-phenyl-1,3,4,6-tetrahydro-5H-benz[f]-2,5-oxazocine

Sodium hydride (24 mg, 1.01 mmol) was added to a solution or desmethylnefopam (200 mg, 0.84 mmol) at RT. The resulting mixture was stirred for 2 hours before allyl bromide (0.12 ml, 1.33 mmol) was added dropwise. The blue solution was heated at 65° C. for a further two hours, then quenched with water. The aqueous mixture was extracted with dichloromethane and the combined extracts dried (MgSO₄), filtered and concentrated in vacuo to provide the target product (66 mg, 28%).

δ_{H} (CDCl₃; 250 MHz) 2.90-3.13 (2H, m, two of CH₂), 3.64 (2H, d, J6.1, two of CH₂), 4.01 (1H, bd, J13.6, one of CH₂), 4.26 (1H, d, J12.1, one of ArCH₂NR₂), 4.38-4.49 (1H, m, one of CH₂), 5.01 (1H, d, J12.1, one of ArCH₂NR₂), 5.42-5.49 (2H, m, two terminal alkenic H), 5.77 (1H, s, CHOR), 6.27-6.37 (1H, m, alkenic H), 7.11-7.39 (8H, aromatic H), 7.51 (1H, bs, aromatic H).

### EXAMPLE 43

### 5-Cyclopropyl-1-phenyl-1,3,4,6-tetrahydro-5H-benz[f]-2,5-oxazocine

Sodium cyanoborohydride (106 mg, 1.69 mmol) was added to a stirring suspension of desmethylnefopam (100 mg, 0.42 mmol), acetic acid (0.24 ml, 4.22 mmol), (1-ethoxycyclopropyloxy)trimethylsilane (0.51 mL, 2.53 mmol) and 4A molecular sieves in methanol (1.5 ml). The resulting brown solution was heated at reflux temperature overnight. The reaction mixture was quenched by the addition of sodium bicarbonate solution and extracted into dichloromethane. The combined organic phases were dried (MgSO₄), filtered and concentrated in vacuo. The crude material was purified by silica gel column chromatography (DCM→2% MeOH/DCM) to provide the target product (84 mg, 68%).

δ_{H} (CDCl₃; 250 MHz) 0.56 (4H, bs, cyclopropyl CH₂CH₂), 1.90-1.94 (1H, m, cyclpropyl CH), 2.88 (1H, ddd, J14.3, 6.1, 2.7, one of CH₂CH₂), 3.00 (1H, ddd, J14.3, 7.3, 2.1, one of CH₂CH₂), 3.87 (1H, ddd, J12.5, 6.1, 2.1, one of CH₂CH₂), 3.96 (1H, d, J12.8, one of ArCH₂NR₂), 4.15 (11 1, ddd, J12.5, 7.3, 2.7, one of CH₂CH₂),4.86 (1H, d, one of ArCH₂NR₂), 5.84 (1H, s, CHOR), 6.99-7.00 (1H, m, aromatic H), 7.18-7.30 (8H, aromatic H).

### EXAMPLE 44

### 5-Propyl-1-phenyl-1,3,4,6-tetrahydro-5H-benz[f]-2,5-oxazocine

5-Allyl-1-phenyl-1,3,4,6-tetrahydro-5H-benz[f]-2,5-oxazocine (66 mg, 0.24 mmol) was dissolved in THF (4 ml) and 10% Pd/C (2 micro-spatulas) added. The reaction vessel was shaken under a hydrogen atmosphere for 3 hours. After this time, the mixture was filtered and the filtrate concentrated in vacuo. Purifcation of the crude mixture by silica gel column chromatography (DCM→5% MeOH/DCM) furnished the target product as a pale yellow oil (42 mg, 62%).

δ_{H} (CDCl₃; 250 MHz)1.03 (3H, t, J7.3, CH₃), 1.99-2.10 (2H, m, two of propyl CH₂CH₂), 2.96-3.22 (4H, m, two of propyl CH₂CH₂ and two of ring CH₂CH₂), 4.02-4.10 (1H, m, ArCH₂NR₂), 4.37-4.51 (2H, m, two of CH₂CH₂) 5.03-5.31(1H, bs, one of ArCH₂NR₂), 5.77 (1H, CHOR), 7.15-7.18 (3H, m, aromatic H), 7.27-7.40 (5H, m, aromatic H), 7.63 (1H, bs, aromatic H).

### EXAMPLE 45

### 5-Methylcyclopropyl-1-phenyl-1,3,4,6-tetrahydro-5H-benz[f]-2,5-oxazocine

Desmethylnefopam (110 mg, 0.43 mmol) was dissolved in anhydrous DMF (2 ml) under a nitrogen atmosphere and sodium hydride (as a 60% suspension in mineral oil, 21 mg, 0.52 mmol) was added. The resulting mixture was heated to 80° C. (Bromomethyl)cyclopropane (0.06 ml, 0.65 mmol) was added and the reaction mixture kept between 80 and 90° C. Once the starting material was shown to have been consumed by TLC, the reaction was quenched by the addition of water and brine. The aqueous mixture was extracted three times with ethyl acetate, and the combined organics were washed with brine, before being dried (MgSO₄), filtered and concentrated in vacuo to give a red/pink oil. Purification by silica gel column chromatography (DCM→1.5% MeOH/DCM) furnished the target product as a red oil (35 mg, 26%).

δ_{H}(CDCl₃; 250 MHz) 0.08-0.20 (2H, m, two of cyclopropyl CH₂CH₂), 0.57-0.63 (2H, m, two of cyclopropyl CH₂CH₂), 0.99-1.04 (1H, m, cyclopropyl CH), 2.41 (1H, dd, J12.5, 7.0, one of CH₂), 2.70 (1H, dd, J12.5, 5.8, one of CH₂), 2.83-3.00 (2H, m, two of CH₂CH₂), 3.90 (1H, ddd, J12.5, 5.8, 2.4, one of CH₂CH₂), 3.95 (1H, d, J12.5, one of ArCH₂NR₂), 4.22 (1H, ddd, J12.5, 7.6, 3.2, one of CH₂CH₂), 4.77 (1H, d, J12.5, one of ArCH₂NR₂), 5.83 (1H, s, CHOR), 7.01 (1H, d, J7.0, aromatic H), 7.16-7.33 (8H, m, aromatic H).

### EXAMPLE 46

### 5-Isopropyl-1-phenyl-1,3,4,6-tetrahydro-5H-benz[f]-2,5-oxazocine

Desmethylnefopam (116 mg, 0.45 mmol) was dissolved in acetone (3 ml) over 4A molecular sieves (spatula tip). A drop of acetic acid was added and the resulting mixture was stirred at RT for one hour. After this time, sodium cyanoborohydrde (114 mg, 1.82 mmol) was added and the mixture was stirred overnight at RT, after which time, TLC showed that starting material had been consumed. The reaction mixture was filtered and the collected solids washed with ethyl acetate. The filtrate was concentrated in vacuo to remove the acetone, providing a pale yellow residue. This was re-dissolved in dichloromethane and washed with a sat. aq. solution of sodium bicarbonate. The organic layer was separated, dried (MgSO₄), filtered and concentrated in vacuo to provide the crude product. Purification by silica gel column chromatography (DCM→1% MeOH/DCM) furnished the desired compound (34 mg, 30%).

δ_{H} (CDCl₃; 250 MHz) 1.13 (3H, d, J6.7, one CH₃ of N(CH₃)₂), 1.25 (3H, d, J6.4, one CH₃ of N(CH₃)₂), 1.73 (1H, dd, J4.6, 1.5, CH), 2.77-3.11 (2H, m, two of CH₂CH₂), 3.71 (1H, d, J12.8, one of ArCH₂NR₂), 3.87 (1H, ddd, J12.2, 8.9, 1.8, one of CH₂CH₂), 4.11 (1H, ddd, J12.2, 5.0, 2.3, one of CH₂CH₂), 4.34 (1H, d, J12.8, one of ArCH₂NR₂), 5.90 (1H, s, CHOR), 7.02 (1H, d, J7.3, aromatic H), 7.16-7.30 (8H, aromatic H).

### EXAMPLE 47

### 8-(4-Pyridyl)-5-methyl-1-(3-methoxy)phenyl-1,3,4,6-tetrahydro-5H-benz[f]-2,5-oxazocine

Compound 21b (117 mg, 0.32 mmol), pyridine-4-boronic acid (52 mg, 0.42 mmol), Pd(PPh₃)₄ (10 mol %) and KOH (2M solution in water, 0.48 ml) were suspended in DME (2 ml). The mixture was degassed and then purged with nitrogen before heating at reflux temperature under a nitrogen atmosphere for 17 hours. The mixture was allowed to cool to RT and diluted with ethyl acetate (20 ml) and washed with water (20 ml). The aqueous phase was extracted into ethyl acetate (2×20 ml) and the combined organic extracts were dried (MgSO₄), filtered and concentrated in vacuo. Purification by silica gel column chromatography (EtOAc→10% MeOH/EtOAc) furnished the target compound as a pale yellow oil (25 mg, 22%).

δ_{H}(CDCl₃; 250 MHz) 2.50 (3H, s, NCH₃), 2.60 (1H, ddd, J14.2, 5.8, 2.6, one of CH₂CH₂), 2.86(1H, ddd, J14.2, 8.3, 2.1, one of CH₂CH₂), 3.75 (1H, d, J12.8, one of ArCH₂NR₂), 3.78 (3H, s, OCH₃), 3.88 (1H, ddd, J12.5, 5.8, 2.1, one of CH₂CH₂), 4.22 (1H, ddd, J12.5, 8.3, 2.6, one of CH₂CH₂), 4.87 (1H, d, J12.8, one of ArCH₂NR₂), 5.81 (1H, s, CHOR), 6.79-6.91 (4H, m, aromatic H), 7.14 (1H, d, J7.6, aromatic H), 7.22-7.28 (1H, m, aromatic H), 7.44-7.62 (4H, m, aromatic H), 8.65 (1H, d, J5.8, aromatic H).

### 5-Pyrimidineboronic acid

ⁿBuLi (3.02 ml, 2M solution in hexane, 7.55 mmol) was added dropwise to a stirring solution of 5-bromopyrimidine (1 g, 6.29 mmol) and triisopropylborate (1.46 ml, 7.55 mmol) in anhydrous toluene (16 ml) and anhydrous THF (4 ml) at −70° C. under a nitrogen atmosphere. The reaction mixture was stirred at −70° C. for 30 mins and then removed from the cold bath. When the internal temp. reached −20° C., the reaction was quenched by the dropwise addition of 2M HCl (10 ml). The mixture was allowed to warm to RT and then separated. The aqueous phase was taken to pH 5.5 with 2M KOH and extracted into THF (3×25 ml). The combined organic extracts were dried (MgSO₄), filtered and concentrated in vacuo to provide a colourless solid. The solid was slurried in acetonitrile (2 ml), collected by filtration and dried on the sinter to give the target boronic acid as a brilliant white solid (340 mg, 44%). δ_{H}(MeOD; 250 MHz) 8.98 (2H, s), 9.14 (1H, s).

### EXAMPLE 48

### 8-(5-Pyrimidine)-5-methyl-1-(3-methoxy)phenyl-1,3,4,6-tetrahydro-5H-benz[f]-2,5-oxazocine

Compound 21b (118 mg, 0.3 mmol), CsF (148 mg, 0.98 mmol), 5-pyrimidine boronic acid (52 mg, 0.42 mmol) and Pd(PPh₃)₄ (10 mol %) were suspended in DME (2 ml) and water (100 μL). The mixture was degassed and purged with nitrogen then heated to reflux temperature for 17 hours. The reaction mixture was allowed to cool to RT before diluting with ethyl acetate (20 ml) arid washing with water (20 ml). The aqueous phase was extracted with ethyl acetate (2×20 ml) and the combined organic extracts were dried (MgSO₄), filtered and evaporated to dryness to provide crude material. Purification by silica gel column chromatography (EtOAc→10% MeOH/EtOAc) furnished the target product as a pale yellow/brown solid (36 mg, 30%).

δ_{H} (CDCl₃; 250 MHz) 2.53 (3H, s, NCH), 2.68 (1H, ddd, J14.2, 5.8, 2.6, one of CH₂CH₂), 2.88 (1H, ddd, J14.2, 8.2, 2.2, one of CH₂CH₂), 3.79 (1H, d, J12.8, one of ArCH₂NR₂), 3.79 (3H, s, OCH₃), 3.89 (1H, ddd, J12.8, 5.8, 2.2, one of CH₂CH₂), 4.24 (1H, ddd, J12.8, 8.2, 2.6, one of CH₂CH₂), 4.89 (1H, d, J12.8, one of ArCH₂NR₂), 5.82 (1H, s, CHOR), 6.79-6.91 (3H, m, aromatic H), 7.17-7.29 (2H, m, aromatic H), 7.40-7.43 (2H, m, aromatic H), 8.95 (s, 2H, pyrimidine H), 9.20 (1H, s, pyrimidine H).

### EXAMPLE 49

### 5-Methyl-1-3-methoxy)phenyl-1,3,4,6-tetrahydro-5H-benz[f]-2,5-oxazocine

Compound 21b (206 mg, 0.57 mmol) was suspended in toluene (3 ml) and degassed under vacuum, then purged with nitrogen. Palladium tetrakis (33 mg) was added to the mixture, which was then subjected to the degassing/nitrogen purge sequence again. A degassed solution of sodium carbonate (2M, 12 ml) and boronic ester (117 mg, 0.57 mmol) were added and the whole mixture was then degassed, purged with nitrogen and heated to 80° C. overnight. The reaction mixture was allowed to cool, diluted with water (10 ml) and the resulting mixture extracted into ethyl acetate (3×50 ml). The combined organic phases were dried (MgSO₄), filtered and concentrated in vacuo to provide the crude product. Purification by silica gel column chromatography [EtOAc] was carried out twice to give a mixture of products.

Mass spec analysis suggested the debrominated species had been formed. m/z (ES⁺) 284 (MH⁺). δ_{H} (CDCl₃; 250 MHz) 2.51 (3H, s, N—CH₃), 2.68 (1H, ddd, J14.2, 5.2, 2.9, one of CH₂CH₂), 2.88 (1H, ddd, J14.2, 9.0, 2.6, one of CH₂CH₂), 3.78 (3H, s, OCH₃), 3.81 (1H, d, J12.8, one of ArCH₂NR₂), 3.88 (1H, ddd, J12.8, 5.2, 2.6, one of CH₂CH₂), 4.23 (1H, ddd, J12.8, 9.0, 2.9, one of CH₂CH₂), 4.86 (1H, d, J12.8, one of ArCH₂NR₂), 5.76 (1H, s, CHOR), 6.78-6.86 (3H, m, aromatic H), 7.03-7.05 (1H, m, aromatic H), 7.20-7.27 (4H, m, aromatic H).

### EXAMPLE 50

### 5-Methyl-1-(3-methoxy)phenyl-1,3,4,6-tetrahydro-5H-benz[f]-2,5-oxazocine-8-carboxamide

Compound 21b (73 mg, 0.24 mmol) was heated to reflux temperature in ^{t}BuOH (3 mL) with KOH (20 mg, 0.36 mmol) for 90 mins. At the end of this time, the reaction mixture was allowed to cool to RT. The mixture was then washed with brine (20 ml) and extracted into dichloromethane (3×20 ml). The combined organic extracts were dried (MgSO₄) and concentrated in vacuo to provide the crude amide as a yellow solid. The solid was slurried in ethyl acetate, collected by suction filtration, washed with heptane and dried on the sinter to provide amide (24 mg, 31%).

δ (MeOD; 250 MHz) 2.44 (3H, s, NCH₃), 2.51-2.60 (1H, m (poorly resolved ddd), one of CH₂CH₂), 2.72-2.81 (1H, m (poorly resolved ddd), one of CH₂CH₂), 3.75 (4H, s, OCH₃ and one of ArCH₂), 3.84-3.90 (1H, m (poorly resolved ddd), one of CH₂CH₂), 4.17-4.27 (1H, m (poorly resolved ddd), one of CH₂CH₂), 5.01 (1H, d, J12.5, one of ArCH₂), 5.83 (1H, s, CHOR), 6.80-6.83 (3H, m, aromatic H), 7.14-7.21 (2H, m, aromatic H), 7.70-7.76 (2H, m, aromatic H). MS (ESI⁻) 325 ([M−H]⁻); (ESI⁺) 349 ([M+Na]⁺)

### 2-Benzoyl-4-fluorobenzoic acid and 2-Benzoyl-5-fluorobenzoic acid

3-Fluorophthalic anhydride (5 g, 33 mmol) was suspended in benzene (17.7 ml) at RT under nitrogen. Aluminium chloride (8.8 g, 66 mmol) was added in portions, turning the mixture orange in colour. The reaction was heated at reflux temperature for 4 hours before cooling to RT. The yellow slurry was added carefully to 10% HCl (100 ml), producing a white precipitate. The aqueous mixture was extracted into ethyl acetate (3×100 mL) and the combined organic extracts were dried (MgSO₄), filtered and concentrated in vacuo to provide a mixture of the keto-acids 2-Benzoyl-4-fluorobenzoic acid and 2-benzoyl-5-fluorobenzoic acid as colourless crystals (8 g, quantitative yield).

δ_{H} (MeOD; 250 MHz) 7.17-7.25 (3H, m, ArH), 7.31-7.42 (2H, m, ArH), 7.48 (2H, d, J8.0, ArH), 7.69 (1H, d, J8.0, ArH).

### 2-Benzoyl-6-fluoro-N-(2-hydroxyethyl)-N-methylbenzamide and 2-Benzoyl-3-fluoro-N-(2-hydroxyethyl)-N-methylbenzamide

A crude mixture of 2-benzoyl-6-fluorobenzoic acid and 2-benzoyl-3-fluorobenzoic acid (7 g, 28 mmol) was suspended in dichloromethane (70 ml) at RT. A few drops of DMF, followed by oxalyl chloride (3 ml, 34 mmol) were added dropwise and the resulting mixture was stirred until gas evolution had ceased, and the suspended solid had dissolved. The mixture was concentrated in vacuo and co-evaporated with dichloromethane (2×40 mL) to provide the crude acid chloride. (2-methylamino)-ethanol (2.24 ml, 28 mmol) and triethylamine (3.9 ml, 28 mmol) were dissolved in dichloromethane (50 ml) and cooled in an ice bath. The acid chloride was dissolved in dichloromethane (50 ml) and added dropwise to the cooled solution of amine. The mixture was allowed to warm to RT and stirred for 4.5 hours, after which point the reaction was quenched by the addition of sat. aq ammonium chloride solution (200 ml). The organics were extracted into dichloromethane (3×100 ml), and the combined extracts were dried (MgSO₄), filtered and concentrated in vacuo. Purification was achieved by gravity silica gel column chromatography (50% EtOAc/heptane to 100% EtOAc) with pre-absorption of the crude amide onto silica.

2-Benzoyl-6-fluoro-N-(2-hydroxyethyl)-N-methylbenzamide (490 mg) was obtained as a 3:4 mixture of two rotamers:

δ_{H} (CDCl₃; 250 MHz) 3.04 (3H, s, CH₃, major rotamer), 3.10 (3H, s, CH₃, minor rotamer), 3.33-3.40 (2H, two of CH₂CH₂, major rotamer), 3.52-3.64 (2H, two of CH₂CH₂, minor rotamer), 3.78-3.96 (4H, two of CH₂CH₂ for major and minor rotamer), 7.22-7.27 (4H, m, ArH both rotamers), 7.28-7.42 (6H, m, ArH both rotamers), 7.55-7.61 (2H, ArH both rotamers), 7.75-7.83 (4H, m, ArH both rotamers).

2-Benzoyl-3-fluoro-N-(2-hydroxyethyl)-N-methylbenzamide (2.74 g) was obtained as two rotamers:

δ_{H} (CDCl₃; 250 MHz) 3.03 (3H, s, CH₃, one rotamer), 3.05 (3H, s, CH₃, one rotamer), 3.55-3.59 (4H, m, two of CH₂CH₂ for each rotamer), 3.75-3.79 (4H, m, two of CH₂CH₂ for each rotamer), 7.16-7.27 (4H, m, ArH both rotamers), 7.44-7.63 (4H, m, ArH both rotamers), 7.84 (2H, d, J7.9, ArH both rotamers).

### 2-{[3-Fluoro-2-(hydroxyphenyl-methyl)-benzyl]-methylamino}-ethanol

2-Benzoyl-3-fluoro-N-(2-hydroxyethyl)-N-methylbenzamide (2.58 g, 8.57 mmol) was dissolved in anhydrous THF (25 ml) at RT under a nitrogen atmosphere. Borane-dimethylsulfide complex (2.0M in THF, 18.8 ml, 37.7 mmol) was added dropwise and the resulting mixture was stirred at RT overnight. The reaction was carefully quenched by the addition of HCl (10%, 36 ml). The mixture was then heated to reflux temperature for approx. 1 hour before cooling to ambient temperature. The THF was removed in vacuo and the remaining solution was partitioned between MTBE (40 ml) and water (60 ml). The aqueous phase was separated, basified with 2M NaOH(aq) and extracted into ethyl acetate (4×50 ml). The combined organic extracts were dried (MgSO₄), filtered and concentrated in vacuo to give 2{[3-fluoro-2-(hydroxyphenyl-methyl)-benzyl]-methylamino}-ethanol as a viscous oil which later crystallised.

δ_{H} (CDCl₃; 250 MHz) 2.17 (3H, s, CH₃), 2.35-2.42 (1H, m, one of CH₂CH₂), 2.52-2.62 (1H, m, one of CH₂CH₂), 2.88 (1H, d, J12.5, one of ArCH₂NR₂), 3.42 (1H, d, J12.5, one of ArCH₂NR₂), 3.62-3.65 (2H, m, two of CH₂CH₂), 6.38 (1H, s, CHOH), 6.96 (1H, d, 17.3, ArH), 7.10-7.37 (7H, m, ArH).

### EXAMPLE 51

### 10-Fluoro-5-methyl-1-phenyl-1,3,4,6-tetrahydro-5H-benz[f]-2,5-oxazocine

Crude 2-{[3-fluoro-2-(hydroxyphenylmethyl)benzyl]methylamino}ethanol (234 mg, 0.81 mmol) was heated to reflux temperature with pTSA (231 mg, 1.21 mmol) in toluene (4 ml). The reaction was kept open to allow the toluene/water to evaporate. After approx. 1.5 hours, a gummy residue remained in the reaction flask. It was allowed to cool to RT at which point, sat. aq sodium bicarbonate solution and ethyl acetate were added to solubilise the residue. The aqueous phase was repeatedly extracted with ethyl acetate, and the combined extracts were dried (MgSO₄), filtered and evaporated to dryness to provide the crude cyclised product. Purification by gravity silica gel column chromatography [EtOAc] furnished the target compound, (90 g, 25%).

δ_{H} (CDCl₃; 250 MHz) 2.47 (3H, s, CH₃), 2.59 (1H, ddd, J13.9, 5.6, 3.3, one of CH₂CH₂), 2.78 (1H, ddd, J13.9, 8.5,2.6, one of CH₂CH₂), 3.58(1H, d, J12.4, one of ArCH₂NR₂), 3.83 (1H, ddd, J12.3, 5.6, 2.6, one of CH₂CH₂), 4.30 (1H, ddd, J12.3, 8.5, 3.3, one of CH₂CH₂), 5.09 (1H, d, J12.4, one of ArCH₂NR₂), 5.94 (1H, d, J1.9, CHOR), 6.89-6.96 (1H, m, ArH), 7.04 (1H, d, J7.6, ArH), 7.19-7.32 (6H, m, ArH)

### 2-{[2-Fluoro-6-(hydroxylphenylmethyl)benzyl]methylamino}ethanol

2-Benzoyl-fluoro-N-(2-hydroxyethyl)-N-methylbenzamide (440 mg, 1.46 mmol) was dissolved in dry THF (4 ml) under nitrogen. Borane-dimethylsulfide (2.0M solution in THF, 3.2 ml, 6.43 mmol) was added dropwise and the resulting mixture was stirred overnight at RT. The reaction was quenched by the addition of 2M HCl (6 ml) and heated at reflux temperature for 1 hour. The acidic mixture was then cooled and partitioned between MTBE (30 ml) and water (30 ml). The aqueous phase was separated and extracted with ethyl acetate (4×30 ml). The combined extracts were dried (MgSO₄), filtered and concentrated in vacuo to give the crude amine. Purification by gravity silica gel column chromatography [EtOAc] furnished 2-{[2-fluoro-6-(hydroxylphenylmethyl)benzyl]methylamino}ethanol (19 mg, 5%).

δ_{H} (250 MHz; CDCl₃) 2.72 (3H, s, NCH₃), 2.54-2.73 (2H, m, two of CH₂CH₂), 3.36 (1H, dd, J12.8, 2.1, one of ArCH₂NR₂), 3.63-3.72 (3H, two of CH₂CH₂ and one of ArCH₂NR₂), 5.91 (1H, s, CHOH), 6.97-7.07 (2H, m, aromatic H), 7.22-7.40 (6H, m, aromatic H).

### EXAMPLE 52

### 7-Fluoro-5-methyl-1-phenyl-1,3,4,6-tetrahydro-5H -benz[f]-2,5-oxazocine

Crude 2-{[2-fluoro-6-(hydroxyphenyl-methyl)benzyl]methylamino}ethanol (20 mg, 0.069 mmol) was dissolved in toluene and pTSA (20 mg, 0.1 mmol) added. The mixture was heated to 120° C. in an open vessel to allow evaporation of the solvent. After 30 mins, a gummy residue remained in the reaction vessel. The vessel was allowed to cool and the residue dissolved in ethyl acetate (5 ml) and sat. aq. sodium bicarbonate solution (10 ml). The aqueous phase was washed with ethyl acetate and the combined organic extracts dried (MgSO₄), filtered and concentrated in vacuo to provide the product as a crude light brown oil (10 mg, 53%).

δ_{H} (CDCl₃, 250 MHz) 2.50 (3H, s, NCH₃), 2.68 (1H, ddd, J13.9, 6.3, 2.8, one of CH₂CH₂), 2.84 (1H, ddd, J13.9, 7.8, 2.1, one of CH₂CH₂), 3.90 (1H, ddd, J 12.5, 6.3, 2.1, one of CH₂CH₂), 4.05 (1H, d, J13.4, one of ArCH₂), 4.21 (1H, ddd, J12.5, 7.8, 2.8, one of CH₂CH₂), 4.71 (b 1H, dd, J13.4, 3.4, one of ArCH₂), 5.81 (1H, s, CHOR), 6.82 (1H, d, J7.6, aromatic H), 7.37 (1H, t, J8.5, aromatic H), 7.10-7.23 (2H, m, aromatic H), 7.25-7.35 (3H, m, aromatic H), 7.37-7.45 (1H, m, aromatic H).

Purity measured at 50%.

### TBS-protected 3-bromophenol

To a solution of 3-bromophenol (100 g, 0.6 mol) in dichloromethane (600 ml) was added imidazole (100 g, 1.46 mol) at 0° C. After 10 min, TBDMS-Cl (96 g, 0.64 mol) was added, and the mixture was stirred at RT overnight. The mixture was then diluted with MTBE (1L) and filtered. The filtrate was washed with sat. ammonium chloride (4×200 ml), sat. bicarb. (200 ml) and brine (200 ml), before being dried (MgSO₄), filtered and concentrated in vacuo to give the product as a yellow oil (156.6 g) that was used without further purification.

δ_{H} (CDCl₃; 250 MHz) 7.10-6.77 (4H, m, aromatics), 0.99 (9H, s, C(CH₃)₃), 0.21 (6H, s, 2×CH₃).

### 4Bromo-2-(3-^{t}butyidimethylsilyloxy-benzoyl)-benzoic acid and 5-Bromo-2-(3-^{t}butydimethylsilyloxy-benzoyl)-benzoic acid

A solution of TBS-protected 3-bromophenol (156.6 g, 0.54 mol) in anhydrous THF (160 ml) was added dropwise to a stirring suspension of magnesium turnings (13 g, 0.54 mol) in dry THF (240 ml). The Grignard formation initiated following the addition of 1 ml of solution. The resulting mixture was left stirring until the reflux had stopped, and then this was added in a single portion to a stirred solution of 4-bromophthalic anhydride (122.4 g, 0.54 mol) in anhydrous THF (240 ml). The reaction mixture was heated at reflux temperature overnight. After cooling to RT, the reaction was quenched by the addition of sat. aq. solution of ammonium chloride (250 ml) and the organics were extracted into ethyl acetate (3×250 ml). The combined organic extracts were dried (MgSO₄), filtered and concentrated in vacuo to provide the crude acid as a brown oil (226.6 g). The crude material was carried through to the next step without further purification.

δ_{H} (CDCl₃; 250MHz) 8.07-6.75 (7H, m, aromatics), 0.99-0.94 (9H, m, C(CH₃)₃), 0.20-0.13 (6H, m, 2×CH₃).

### 5-Bromo-N-(2-hydroxyethyl)-N-methyl-2-3-^{t}butyidimethylsilyloxy-benzoyl)-benzamide

To a solution of a mixture of 4-bromo-2-(3-^{t}butyldimethylsilyloxy-benzoyl)-benzoic acid and 5-bromo-2-(3-^{t}butyidimethylsilyloxy-benzoyl)-benzoic acid (226.6 g, 0.52 mol) in anhydrous dichloromethane (900 ml), under an atmosphere of nitrogen, was added anhydrous N,N-dimethylformamide (1 ml) followed by the slow addition of oxalyl chloride (50.8 ml, 0.57 mol), and the reaction stirred at RT overnight. The dichloromethane was then removed in vacuo. Dichloromethane (2×500 ml) was added to the crude oil and evaporated in vacuo. The crude acid chloride was dissolved in dichloromethane (900 ml), cooled to 0° C. (ice bath) and triethylamine (81 ml, 0.57 mol) added dropwise. N-methylethanolamine (42.8 g, 0.57 mol) was then added dropwise and the mixture left to warm to RT overnight under an atmosphere of nitrogen. The mixture was then quenched with brine (800 ml) and the layers separated. The aqueous layer was then separated and washed with dichloromethane (800 ml), and the combined organics were dried (MgSO₄), filtered and concentrated in vacuo to yield the mixture of regioisomers (255.5 g) as a dark brown oil.

100 g of the above brown oil was purified by silica gel column chromatography (3:1 EtOAc:Heptane). This afforded 14 g of 5-bromo-N-(2-hydroxyethyl)-N-methyl-2-(3-^{t}butyldimethylsilyloxy-benzoyl)-benzamide.

δ_{H} (CDCl₃; 250 MHz) 7.59-7.25 (7H, m, aromatics), 3.91-3.57 (4H, m, CH₂—CH₂), 3.11 and 2.99 (3H, 2×s, NCH₃), 0.98 (9H, s, C(CH₃)₃), 0.21 (6H, s, 2×CH.

### 5-Bromo-N-(2-hydroxyethyl)-N-methyl-2-(3-tosyloxy-benzoyl )-benzamide

TBAF (10.1 ml, 1M solution in THF, 10.14 mmol) was added to a stirred solution of 5-bromo-N-(27hydroxyethyl)-N-methyl-2-(3-^{t}butyldimethylsilyloxy-benzoyl)-benzamide (5 g, 10.14 mmol) in THF (60 ml) at RT. A solution of tosyl chloride (1.9 g, 10.14 mmol) in THF (60 ml) was then added dropwise and the mixture heated to reflux temperature for 3 h. A further 10 mmol of triethylamine followed by 7 mmol of TsCl were added over a further hour to drive the reaction is to completion. After cooling to RT the mixture was diluted with ethyl acetate (100 ml), washed with water (2×100 ml), dried (MgSO₄), filtered and concentrated in vacuo to give 7 g of an orange oil. Purification by silica gel column chromatography [2:1 EtOAc:Heptane] afforded the title compound (3.14 g, 58%) as a pale yellow oil.

δ_{H} (CDCl₃; 250 MHz) 7.74-7.20 (11H, m, aromatics), 3.86-3.53 (4H, m, CH₂CH₂), 3.07 and 2.97 (3H, 2×s, NCH₃), 2.47 (3H, s, Ar—CH×₃).

### 2-({5-Bromo-2-[hydroxyl-(3-tosyloxy-phenyl)-methyl]-benzyl}-methylamino)-ethanol

Borane dimethylsulfide complex (2.0M in THF, 12.94 ml, 25.88 mmol) was added dropwise to a solution of 5-bromo-N-(2-hydroxyethyl)-N-methyl-2-(3-tosyloxy-benzoyl)-benzamide (3.14 g, 5.88 mmol) in anhydrous THF (20 ml) at 0° C. The reaction mixture was left to stir at RT overnight. The crude mixture was then quenched carefully by addition of an aqueous 6M HCl solution (20 ml) and then heated to reflux temperature for 2 h. After cooling to RT, the THF was removed in vacuo. Water (10 ml) was added and the mixture was then basified to pH 10 by addition of an aqueous solution of sodium hydroxide solution (50%), and then extracted with ethyl acetate (2×20 mL). The combined organic layers were then dried (MgSO₄), filtered and concentrated in vacuo to give a white oil/solid (3.17 g). Purification by silica gel column chromatography [5:1, EtOAc:Heptane] afforded the title compound (0.74 g, 24%) as a colourless oil.

δ_{H} (CDCl₃; 250 MHz) 7.67-6.83 (11H, m, aromatics), 5.72 (1H, s, CHOH), 3.73-3.67 (2H, m, CH₂), 3.39-3.27 (2H, m, CH₂), 2.63-2.53 (2H, m, CH₂), 2.44 (3H, s, Ar—CH₃), 2.22 (3H, s, NCH₃).

### EXAMPLE 53

### 8-Bromo-5-methyl-1-(3-tosyloxy)phenyl-1,3,4,6-tetrahydro-5H-benz[f]-2,5-oxazocine

2-({5-Bromo-2-[hydroxyl-(3-tosyloxyphenyl)methyl]benzyl}-methylamino)ethanol (0.74 g, 1.43 mmol) was heated to reflux temperature under Dean-Stark conditions with pTSA (0.41 g, 2.14 mmol) in toluene (20 ml) for 2 h. The reaction mixture was allowed to cool and then diluted with ethyl acetate (10 ml). The mixture was washed with sat. aq. sodium bicarbonate solution (2×10 ml). The combined organic extracts were dried (MgSO₄), filtered and concentrated in vacuo to provide 0.65 g of product that was used without further purification.

δ_{H} (CDCl₃; 250 MHz) 7.67-6.72 (11H, m, aromatics), 5.67 (1H, s, CH—O), 4.55 (1H, d, J 13, ArCH_{A}H_{B}N), 4.11 (1H, ddd, J 13, 8 and 2.5, CH_{A}H_{B}CH₂), 3.83 (1H, ddd, J 13, 8 and 2.5, CH_{A}H_{B}CH₂), 3.60 (1H, d, J 13, ArCH_{A}H_{B}N), 2.79-2.59 (2H, m, CH₂CH₂), 2.48 and 2.46 (6H, 2×s, 2×CH₃).

### EXAMPLE 54

### 8-Bromo-5-methyl-1-(3-hydroxy)phenyl-1,3,4,6-tetrahydro-5H-benz[f]-2,5-oxazocine

Potassium hydroxide (0.30 g, 5.33 mmol) was added to a stirred solution of 8-bromo-5-methyl-1-(3-tosyloxy)phenyl-1,3,4,6-tetrahydro-5H-benz[f]-2,5-oxazocine (0.65 g, 1.3 mmol) in MeOH (5 ml) and DMF (5 ml) and the mixture heated at 60° C. for 3 h. After cooling to RT, the crude mixture was poured into water (10 ml) and the mixture extracted with dichloromethane (2×10 ml). The aqueous layer was then acidified to pH 9 and extracted with dichloromethane (2×10 ml). The combined organics were dried (MgSO₄), filtered and concentrated in vacuo to give 0.42 g of a yellow oil. Purification by silica gel column chromatography (5% MeOH/DCM) afforded the title compound (0.20 g, 44%) as a white solid.

δ_{H} (CDCl₃; 250 MHz) 7.34-6.67 (7H, m, aromatics), 5.60 (1H, s, CH—O), 517 (1H, d, J 13, ArCH_{A}H_{B}N), 4.20 (1H, ddd, J 13, 8 and 2.5, CH_{A}H_{B}CH₂), 3.80 (1H, ddd, J 13, 8 and 2.5, CH_{A}H_{B}CH₂), 3.53 (1H, d, J 13, ArCH_{A}H_{B}N), 2.75-2.56 (2H, m, CH₂CH₂), 2.36 (3H, s, NCH₃).

### EXAMPLE 55

### 8-Cyano-5-methyl-1-(3-hydroxy)phenyl-1,3,4,6-tetrahydro-5H-benz[f]-2,5-oxazocine

8-Bromo-5-methyl-1-3-hydroxy)phenyl-1,3,4,6-tetrahydro-5H-benz[f]-2,5-oxazocine (100 mg, 0.29 mmol), Zn (1 mg, 0.02 mmol), Zn(OAc)₂ (7 mg, 0.034 mmol), Zn(CN)₂ (25 mg, 0.22 mmol), Pd₂(dba)₃ (37 mg, 0.04 mmol) and 1,1′-bis(diphenylphosphino)ferrocene (18 mg, 0.034 mmol) were heated to 140° C. in DMF (3 ml) and H₂O (30 μl) for 4 h. The black reaction mixture was allowed to cool to RT and diluted with water (20 ml). The resulting brown precipitate was collected by filtration and washed with water then brine. The filtrate was extracted with ethyl acetate (2×60 ml). The combined organic extracts were dried (MgSO₄), filtered and concentrated in vacuo to provide the crude product as a brown oil. Purification by gravity silica gel column chromatography [dichloromethane] furnished the desired product (25 mg, 29%) as a yellow oil.

δ_{H}(CDCl₃; 250 MHz) 7.45-6.70 (7H, m, aromatics), 5.65 (1H, s, CHO), 5.03 (1H, d, J 13, ArCH_{A}H_{B}—N), 4.26-4.18 (1H, m, CH_{A}H_{B}CH₂), 3.81-3.75 (1H, m, CH_{A}H_{B}CH₂), 3.61 (1H, d, J 13, ArCH_{A}H_{B}—N), 2.70-2.53 (2H, m, CH₂CH₂), 2.41 (3H, s, NCH₃).

### EXAMPLE 56

### 8-Cyclopropyl-5-methyl-1-(3-hydroxy)phenyl-1,3,4,6-tetrahydro-5H-benz[f]-2,5-oxazocine

8-Bromo-5-methyl-1-(3-hydroxy)phenyl-1,3,4,6-tetrahydro-5H-benz[f]-2,5-oxazocine (100 mg, 0.29 mmol), cyclopropylboronic acid (33 mg, 0.36 mmol), K₃PO₄ (217 mg, 1.01 mmol), Pd(OAc)₂(4 mg, 0.015 mmol) and P(cyclohexane)₃ (9 mg, 0.029 mmol) were heated to 100° C. in toluene (2.5 mL) and H₂O (125 μl) for 4 hours. After cooling to RT, water (2.5 ml) was added and the mixture extracted with ethyl acetate (2×5 ml), washed with brine, dried (MgSO₄), filtered and concentrated in vacuo. Purification by gravity silica gel column chromatography[dichloromethane] furnished the desired product (40 mg, 44%) as a colourless oil.

δ_{H} (CDCl₃; 250 MHz) 7.27-6.69 (7H, m, aromatics), 5.61 (1H, s, CHO), 5.04 (1H, d, J 13, ArCH_{A}H_{B}—N) 4.23-4.15 (1H, m, CH_{A}H_{B}CH₂), 3.83-3.78 (1H, m, CH_{A}H_{B}CH₂), 3.66 (1H, d, J 13, ArCH_{A}H_{B}—N), 2.78-2.58 (2H, m, CH₂CH₂), 2.41 (3H, s, NCH₃), 1.84-1.78 (1H, m, CHCH₂), 0.95-0.90 (2H, m, CH₂CH₂), 0.64-0.62 (2H, m, CH₂CH₂).

### EXAMPLE 57

### 8-Bromo-5-methyl-1-(3-allyloxy)phenyl-1,3,4,6-tetrahydro-5H-benz[f]-2,5-oxazocine

8-Bromo-5-methyl-1-(3-hydroxy)phenyl-1,3,4,6-tetrahydro-5H-benz[f]-2,5-oxazocine (50 mg, 0.14 mmol), allyl alcohol (33 mg, 0.57 mmol), Pd(OAc)₂ (1 mg, 0.0035 mmol), triphenylphosphine (2 mg, 0.0075 mmol), Ti(OⁱPr)₄ (10 mg, 0.035 mmol), molecular sieves (4 Å, 28 mg) were heated at 50° C. in benzene (1 ml) overnight. After cooling to RT, the black reaction mixture poured into water (5 ml), extracted with MTBE (2×5 ml). The combined organic extracts were dried (MgSO₄), filtered and concentrated in vacuo to provide the crude product as a brown oil. Purification by gravity silica gel column chromatography[dichloromethane] furnished the desired product (16 mg, 30%) as a pale yellow oil.

δ_{H}(CDCl₃; 250 MHz) 7.38-7.19 (3H, m, aromatics), 6.90-6.80 (4H, m, aromatics), 6.09-5.96 (1H, m, CH═CH₂), 5.71 (1H, s, CHO), 5.42-5.25 (2H, m, CH═CH₂), 4.76 (1H, d, J 13, ArCH_{A}H_{B}—N), 4.51 (2H, d, J 5, OCH₂), 4.21-4.12 (1H, m, CH_{A}H_{B}CH₂), 3.88-3.80 (1H, m, CH_{A}H_{B}CH₂), 3.63 (1H, d, J 13, ArCH_{A}H_{B}—N), 2.87-2.77 (1H, m, CH₂CH_{A}H_{B}), 2.69-2.60 (1H, m, CH₂CH_{A}H_{B}) 2.47 (3H, s, NCH₃).

### EXAMPLE 58

### 8-Cyano-5-methyl-1-(3-allyloxy)phenyl-1,3,4,6-tetrahydro-5H-benz[f]-2,5-oxazocine

8-Cyano-5-methyl-1-(3-hydroxy)phenyl-1,3,4,6-tetrahydro-5H-benz[f]-2,5-oxazocine (50 mg, 0.17 mmol), allyl alcohol (49 mg, 0.68 mmol), Pd(OAc)₂ (1 mg, 0.0035 mmol), triphenylphosphine (2 mg, 0.0075 mmol), Ti(OⁱPr)₄ (20 mg, 0.07 mmol), molecular sieves (4 Å, 40 mg) were heated at 50° C. in benzene (2 ml) overnight. After cooling to RT, the black reaction mixture poured into water (5 ml) and extracted with MTBE (2×5 ml). The combined organic extracts were dried (MgSO₄), filtered and concentrated in vacuo to provide the crude product as a brown oil. Purification by gravity silica gel column chromatography[dichloromethane] furnished the desired product (4 mg, 7%) as a pale yellow oil.

δ_{H}(CDCl₃; 250 MHz) 7.65-6.81 (7H, m, aromatics), 6.08-5.97 (1H, m, CH═CH₂), 5.74 (1H, s, CHO), 5.42-5.25 (2H, m, CH═CH₂), 4.98 (1H, d, J 13, ArCH_{A}H_{B}—N), 4.51 (2H, d, J 5, OCH₂), 4.31-4.23 (1H, m, CH_{A}H_{B}CH₂), 3.90-3.82 (1H, m, CH_{A}H_{B}CH₂), 3.74 (1H, d, J 13, ArCH_{A}H_{B}—N), 2.81-2.70 (2H, m, CH₂CH₂), 2.51 (3H, s, NCH₃).

### EXAMPLE 59

### 8-Cyclopropyl-5-methyl-1-(3-allyloxy)phenyl-1,3,4,6-tetrahydro-5H-benz[f]-2,5-oxazocine

8-Cyclopropyl-5-methyl-1-(3-hydroxy)phenyl-1,3,4,6-tetrahydro-5H-benz[f]-2,5-oxazocine (90 mg, 0.29 mmol), allyl alcohol (0.1 ml, 1.16 mmol), Pd(OAc)₂ (1 mg, 0.0035 mmol), triphenylphosphine (3 mg, 0.011 mmol), Ti(OⁱPr)₄ (0.03 mL, 0.07 mmol), molecular sieves (4 Å, 64 mg) were heated at 50° C. in benzene (3 ml) overnight. After cooling to RT, the black reaction mixture was poured into water (5 ml), extracted with MTBE (2×5 ml). The combined organic extracts were dried (MgSO₄), filtered and concentrated in vacuo to provide the crude product as a brown oil. Purification by gravity silica gel column chromatography[dichloromethane] furnished the desired product (3 mg, 3%) as a pale yellow oil.

δ_{H}(CDCl₃; 250 MHz) 7.24-6.76 (7H, m, aromatics), 6.04-5.97 (1H, m, CH═CH₂), 5.41 (1H, s, CHO), 5.41-5.24 (2H, m, CH═CH₂), 4.70 (1H, d, J 13, ArCH_{A}H_{B}—N), 4.49 (2H, d, J 5, OCH₂), 4.19-4.11 (1H, m, CH_{A}H_{B}CH₂), 3.88-3.82 (1H, m, CH_{A}H_{B}CH₂), 3.71 (1H, d, J 13, ArCH_{A}H_{B}—N), 2.92-2.82 (1H, m, CH₂CH_{A}H_{B}), 2.69-2.66 (1H, m, CH₂CH_{A}H_{B}), 2.49 (3H, s, NCH₃), 1.89-1.83 (1H, m, CHCH₂), 0.98-0.90 (2H, m, CHCH₂), 0.71-0.65 (2H, m, CHCH₂).

The following Examples were prepared in a similar manner.

The substituted ‘bromophenyl’ starting materials, used for the Grignard formation, were either sourced commercially or generated synthetically via known organic chemistry, e.g. as in the following example where a nitro group was converted to a bromo via the intermediate amine.

### 2-Methoxy6-aminotoluene

2-methyl-3-nitroanisole (20 g, 0.12 mol) was suspended in methanol (200 ml) at RT under nitrogen. Pd/C (5%, 2 g) was added and the system was shaken under an atmosphere of hydrogen overnight, until TLC indicated one product. The reaction mixture was filtered through celite, and the celite washed with methanol (3×100 ml) and the filtrate concentrated in vacuo. This afforded 18 g of a red oil that was used without further purification.

δ_{H} (CDCl₃; 250 MHz) 7.01 (1H, t, J 8, aromatics), 6.38 (2H, d, J 8, aromatics), 3.83 (3H, s, OCH₃), 2.08 (3H, s, CH₃).

### 2-Methoxy4-bromotoluene

Crude 2-methoxy-6-amino-toluene (max. 120 mmol) was suspended in HBr (48%, 48 ml) and water (120 ml) and cooled to 0° C. in an ice bath. Sodium nitrite (9.2 g) in water (24 ml) was added dropwise to the cold mixture, which turned yellow then brown. After 10 min, excess nitrite was destroyed by addition of urea (0.08 g) and the mixture was rapidly filtered into cold (0° C.) acetone (480 ml) to give a bright yellow solution. CuBr (99.999%, 18.89, 131 mmol) was then added portionwise and the resulting mixture stirred at 0° C. for 3 h. Gas evolution was observed. The mixture was allowed to warm to ambient temperature, and then concentrated in vacuo. Dichloromethane was then added and the mixture washed with sat. aq. sodium bicarbonate solution. The organic layer was separated, dried over magnesium sulphate and concentrated in vacuo to give the product (23.1 g, 96%) as a red oil.

δ_{H} (CDCl₃; 250 MHz) 7.18-6.77 (3H, m, aromatics), 3.83 (3H, s, OCH₃), 2.32 (3H, s, CH₃). 5-Bromo-2-(3-methoxy-2-methylbenzoyl)-benzoic acid and 4-Bromo-2-(3-methoxy-2-methylbenzoyl)-benzoic acid

2-methoxy-4-bromo-toluene (23.1 g, 115 mmol) in dry THF (35 ml) was added dropwise to a stirred suspension of magnesium turnings (2.77 g, 115 mmol) and a crystal of iodine in dry THF (40 ml) under a nitrogen atmosphere. Upon addition, the reaction mixture reached reflux. After cooling to RT, this Grignard solution was added dropwise to a stirred solution of 4-bromophthalic anhydride (26.1 g, 115 mmol) in dry THF (45 ml) and the resulting mixture heated to reflux temperature overnight. After cooling, the reaction was quenched with sat. aq. ammonium chloride solution, and extracted with ethyl acetate. The organics were dried (MgSO₄), filtered and concentrated in vacuo to give the crude mixture of keto-acids (34 g) as a brown solid. This material was carried straight on to the subsequent step.

### 5-Bromo-N-(2-hydroxyethyl)-N-methyl-2-(3-methoxy-2-methyl-benzoyl)-benzamide

A crude mixture of 5-bromo-2-(3-methoxy-2-methylbenzoyl)-benzoic acid and 4-bromo-2-(3-methoxy-2-methylbenzoyl)-benzoic acid (34 g, 98.5 mmol) was dissolved in dichloromethane (200 ml) at RT. DMF (1 ml) followed by oxalyl chloride (9.67 ml, 13.98 g, 108.35 mmol) were added dropwise and the reaction stirred at RT overnight. The reaction mixture was then concentrated in vacuo and co-evaporated with dichloromethane (2×200 ml) to provide the crude acid chloride. This was dissolved in dichloromethane (200 ml), cooled to 0° C., and triethylamine (15.35 ml, 11.22 g, 108.35 mmol) was added dropwise, followed by N-methylethanolamine and the mixture left to warm to RT overnight. The reaction was then quenched with brine and extracted with dichloromethane. The combined organic extracts were dried (MgSO₄), filtered and concentrated in vacuo to give the crude amide as a mixture of regioisomers. The 5-bromo-N-(2-hydroxyethyl)-N-methyl-2-(3-methoxy-2-methyl-benzoyl)-benzamide (10 g, 25%) was separated by silica gel column chromatography [4:1 EtOAc/heptane]. δ_{H}(CDCl₃; 250 MHz) 7.55-6.91 (6H, m, aromatics), 3.98-3.69 (7H, m, OCH₃ and CH₂CH₂), 3.15 and 2.99 (3H, 2×s, NCH₃), 2.20 and 2.17 (3H, 2×s, CH₃).

### 2-({5-Bromo-2-[hydroxyl-(3-methoxy-2-methylphenyl)methyl]benzyl}methylamino)-ethanol

Borane-dimethyl sulphide complex (2.0 M, 55 ml, 0.11 mol) was added dropwise to a stirring solution of 5-bromo-N-(2-hydroxyethyl)-N-methyl-2-3-methoxy-2-methyl-benzoyl)-benzamide (10 g, 0.025 mol) in anhydrous THF (80 ml) at RT under nitrogen, and allowed to stir overnight. The reaction was quenched by the careful addition of 6M HCl (80 ml), and the resulting mixture was heated to reflux temperature for 2 hours. After cooling to RT, the THF was removed in vacuo and the mixture partitioned between water (40 ml) and MTBE (80 ml). The aqueous phase was separated, basified with aq. 2M NaOH, and extracted into ethyl acetate (3×90 ml). The combined organic extracts were dried (MgSO₄), filtered and concentrated in vacuo to provide the desired amine as a viscous oil, which slowly crystallised (6.2 g, 63%). The material was used in the next step without further purification.

δ_{H} (CDCl₃; 250 MHz) 7.41-7.25 (4H, m, aromatics), 6.89 (1H, d, J8, aromatics), 6.61 (1H, d, J8, aromatics), 6.10 (1H, s, CHOH), 4.33 (1H, d, J 13, CH_{A}H_{B}N), 3.84 (3H, s, OCH₃), 3.84-3.62 (2H, m, CH₂), 3.26 (1H, d, J 13, CH_{A}H_{B}N), 2.77-2.73 (2H, m, CH₂), 2.33 (3H, s, NCH₃), 1.83 (3H, s, CH₃).

### EXAMPLE 60

### 8-Bromo-5-methyl-1-(2-methyl-3-methoxy)phenyl-1,3,4,6-tetrahydro-5H-benz[f]-2,5-oxazocine

2-({5-bromo-2-[hydroxyl-(3-methoxy-2-methylphenyl)-methyl]-benzyl}-methylamino)-ethanol (5 g, 12.6 mmol) was heated to reflux temperature in toluene (250 ml) with pTSA (3.65 g, 18.95 mmol) under Dean-Stark conditions for 4 hours. The reaction mixture was allowed to cool to RT, diluted with ethyl acetate (200 ml) and washed with a sat. aq. solution of sodium bicarbonate (300 ml). The aqueous layer was extracted with ethyl acetate (3×200 ml) and the combined organic extracts were dried over magnesium sulfate, filtered and concentrated in vacuo. Purification by silica gel column chromatography [DCM] gave the target product as a pale straw coloured oil (2.8 g, 59%).

δ_{H} (CDCl₃; 250 MHz) 7.42-6.70 (6H, m, aromatics), 5.97 (1H, s, CHO), 4.69 (1H, d, J 13, CH_{A}H_{B}N), 4.14-4.05 (1H, m, CH_{A}H_{B}), 3.89-3.73 (5H, m, CH_{A}H_{B}, OCH₃, and CH_{A}H_{B}N), 2.92-2.82 (1H, m, CH_{A}H_{B}), 2.69-2.59 (1H, m, CH_{A}H_{B}), 2.45 (3H, s, CH₃N), 2.15 (3H, s, CH₃).

### EXAMPLE 61

### 8-Cyano-5-methyl-1-(2-methyl-3-methoxy)phenyl-1,3,4,6-tetrahydro-5H-benz[f]-2,5-oxazocine

8-Bromo-5-methyl-1-(2-methyl-3-methoxy)phenyl-1,3,4,6-tetrahydro-5H-benz[f]-2,5-oxazocine (250 mg, 0.66 mmol), Zn (4 mg, 0.04 mmol), Zn(OAc)₂ (14 mg, 0.07 mmol), Zn(CN)₂ (50 mg, 0.44 mmol), 1,1′-bis(diphenylphosphino)ferrocene (36 mg, 0.07 mmol) and Pd₂(dba)₃ (74 mg, 0.08 mmol) were heated to 140° C. in degassed DMF (3 ml) and H₂O (30 μl) overnight. The mixture was allowed to cool to RT and water (10 ml) was added, producing a brown precipitate. The precipitate was collected by filtration and washed with water and brine. The filtrate was then extracted with ethyl acetate (3×20 mL). The combined organic extracts were dried (MgSO₄), filtered and concentrated in vacuo to provide 100 mg of the crude product. Purification by gravity silica gel column chromatography [DCM] furnished the target product as a yellow oil (70 mg, 33%).

δ_{H} (CDCl₃; 250 MHz) 7.60 (1H, d, J 1.5, aromatics), 7.48 (1H, dd, J 1.5 and 8, aromatics), 7.12-7.01 (2H, m, aromatics), 6.82 (1H, d, J8, aromatics), 6.46 (1H, d, J 8, aromatics), 5.97 (1H, s, CHO), 5.02 (1H, d, J 13, CH_{A}H_{B}N), 4.30-4.24 (1H, m, CH_{A}H_{B}CH₂), 3.96-3.80 (2H, m, CH_{A}H_{B}N and CH_{A}H_{B}CH₂), 3.82 (3H, s, OCH₃), 2.96-2.85 (1H, m, CH₂CH_{A}H_{B}), 2.76-2.65 (1H, m, CH₂CH_{A}H_{B}), 2.53 (3H, s, NCH₃), 2.20 (3H, s, CH₃).

### EXAMPLE 62

### 8-Cyclopropyl-5-methyl-1-(2-methyl-3-methoxy)phenyl-1,3,4,6-tetrahydro-5H-benz[f]-2,5-oxazocine

8-Bromo-5-methyl-1-(2-methyl-3-methoxy)phenyl-1,3,4,6-tetrahydro-5H-benz[f]-2,5-oxazocine (295 mg, 0.71 mmol), cyclopropylboronic acid (89 mg, 0.96 mmol), P(cyclohexane)₃ (24 mg, 0.08 mmol), K₃PO₄ (586 mg, 2.73 mmol) and Pd(OAc)₂ (10.4 mg, 0.04 mmol) were heated at 100° C. in toluene (7 ml) and water (336 μl) overnight. The reaction mixture was allowed to cool and then diluted with ethyl acetate (30 ml). The organic mixture was washed with water (30 ml) and the aqueous phase extracted into ethyl acetate (3×20 ml). The combined organic extracts were dried (MgSO₄), filtered and concentrated in vacuo to provide 0.29 g of the crude product. Purification by silica gel gravity column chromatography [DCM] furnished the target product (100 mg, 38%) as a yellow oil/solid.

δ_{H} (CDCl₃; 250 MHz) 7.11 (1H, t, J 8, aromatics), 6.97 (1H, d, J 1.5, aromatics), 6.88-6.71 (4H, m, aromatics), 5.98 (1H, s, CHO), 4.63 (1H, d, J 13, CH_{A}H_{B}N), 4.18-4.06 (1H, m, CH_{A}H_{B}CH₂), 3.89-3.82 (5H, m, CH_{A}H_{B}N, CH_{A}H_{B}CH₂ and OCH₃), 2.98-2.88 (1H, m, CH₂CH_{A}H_{B}), 2.72-2.64 (1H, m, CH₂CH_{A}H_{B}), 2.50 (3H, s, NCH₃), 2.16 (3H, s, CH₃), 1.91-1.84 (1H, m, CHCH₂), 0.99-0.91 (2H, m, CH₂CH₂), 0.73-0.66 (2H, m, CH₂CH₂).

### EXAMPLE 63

### 8-Bromo-5-methyl-1-(3-methoxy-4-methyl)phenyl-1,3,4,6-tetrahydro-5H-benz[f]-2,5-oxazocine

2-({5-bromo-2-[hydroxyl-(3-methoxy-4-methylphenyl)-methyl]-benzyl}-methylamino)-ethanol (879 mg, 2.23 mmol) was heated to reflux temperature in toluene (9 ml) with pTSA (635 mg, 3.34 mmol) under Dean-Stark conditions for 3 hours. The reaction mixture was allowed to cool to RT, diluted with ethyl acetate (50 ml) and washed with a saturated solution of sodium bicarbonate (60 ml). The aqueous layer was extracted into ethyl acetate (3×50 ml) and the combined organic extracts were dried (MgSO₄), filtered and concentrated in vacuo. Purification by gravity silica gel column chromatography [EtOAc] gave the target product as a pale straw coloured oil (23 g, 28%). A second impure batch of product was obtained from the column.

δ_{H} (CDCl₃; 250 MHz) 2.18 (3H, s, CH₃), 2.48 (3H, s, NCH₃), 2.66 (1H, ddd, J14.2, 5.6, 2.7, one of CH₂CH₂), 2.84 (1H, ddd, J14.2, 8.2, 2.1, one of CH₂CH₂), 3.67 (1H, d, J12.8, one of ArCH₂NR₂), 3.80 (3H, s, OCH₃), 3.86 (1H, ddd, J12.7, 5.6, 2.1, one of CH₂CH₂), 4.18 (1H, ddd, J12.8, 8.2, 2.1, one of CH₂CH₂), 4.80 (1H, d, J12.8, one of ArCH₂NR₂), 5.71 (1H, s, CHOR), 6.68 (1H, dd, J7.6, 1.2, one of ArH), 6.76 (1H, bs, one of ArH), 6.89 (1H, d, J 8.2, ArH), 7.06 (1H, d, J7.6, one of ArH), 7.32 (1H, dd, J8.2, 1.8, one of ArH), 7.39 (1H, d, J1.8, one of ArH).

### EXAMPLE 64

### 8-Cyano-5-methyl-1-(3-methoxy-4-methyl)phenyl-1,3,4,6-tetrahydro-5H-benz[f]-2,5-oxazocine

8-Bromo-5-methyl-1-(3-methoxy-4-methyl)phenyl-1,3,4,6-tetrahydro-5H-benz[f]-2,5-oxazocine (154 mg, 0.41 mmol), Zn (2 mg, 0.03 mmol), Zn(OAc)₂ (10 mg, 0.05 mmol), Zn(CN)₂ (29 mg, 0.25 mmol), 1,1′-bis(diphenylphosphino)ferrocene (30 mg, 0.05 mmol) and Pd₂(dba)₃ (55 mg, 0.06 mmol) were heated to 140° C. in DMF (3 ml) and H₂O (30 μl) overnight. The mixture was allowed to cool to RT and water (30 ml) was added, producing a brown precipitate. The precipitate was collected by filtration and washed with water and ethyl acetate. The filtrate was then washed with brine (2×40 ml) and the combined aqueous phases were extracted with ethyl acetate (3×20 ml). The combined organic extracts were dried (MgSO₄), filtered and concentrated in vacuo to provide the crude product. Purification by silica gel gravity column chromatography [EtOAc] furnished the target product as a brown oil (75 mg, 57%).

δ_{H} (CDCl₃; 250 MHz) 2.17 (3H, s, CH₃), 2.46 (3H, s, NCH₃), 2.65 (1H, ddd, J14.3, 5.1, 3.0, one of CH₂CH₂), 2.78 (1H, ddd, J14.3, 8.6, 2.5, one of CH₂CH₂), 3.71 (1H, d, J13.0, one of ArCH₂NR₂), 3.79 (3H, s, OCH₃), 3.84 (1H, ddd, J12.8, 5.1, 2.5, one of CH₂CH₂), 4.26 (1H, ddd, J12.8, 8.6, 3.0, one of CH₂CH₂), 4.99 (1H, d, J13.0, one of ArCH₂NR₂), 5.73 (1H, CHOR), 6.63 (1H, d, J1.5, ArH), 6.72 (1H, s, ArH), 7.06 (1H, d, J7.6, ArH), 7.14 (1H, d, J8.2, ArH), 7.47 (1H, dd, J8.2, 1.8, ArH), 7.53 (1H, d, J1.5, ArH).

### EXAMPLE 65

### 5-Methyl-1-(3-methoxy-4-methyl)phenyl-1,3,4,6-tetrahydro-5H-benz[f]-2,5-oxazocine-8-carboxamide

8-Cyano-5-methyl-1-(3-methoxy-4-methyl)phenyl-1,3,4,6-tetrahydro-5H-benz[f]-2,5-oxazocine (1.1 g, 3.56 mmol) was heated to reflux temperature in ^{t}BuOH (24 ml) with potassium hydroxide (0.29 mg, 5.34 mmol) for 2 h. At the end of this time, the reaction mixture was allowed to cool to RT. The mixture was then washed with brine (50 ml) and extracted into dichloromethane (3×50 ml). The combined organic extracts were dried (MgSO₄), filtered and concentrated in vacuo to provide the crude amide as a light brown solid. Purification by silica gel column chromatography [1% MeOH in DCM to 12% MeOH in DCM] afforded the title compound (385 mg, 32%).

δ (CDCl₃; 250 MHz) 2.16 (3H, s, CH₃), 2.47 (3H, s, NCH₃), 2.52-2.62 (1H, m (poorly resolved ddd), one of CH₂CH₂), 2.75-2.88 (1H, m (poorly resolved ddd), one of CH₂CH₂), 3.77 (4H, s, OCH₃ and one of ArCH₂), 3.81-3.92 (1H, m (poorly resolved ddd), one of CH₂CH₂), 4.15-4.28 (1H, m (poorly resolved ddd), one of CH₂CH₂), 4.89 (1H, d, J12.8, one of ArCH₂), 5.77 (1H, s, CHOR), 6.76-6.75 (3H, m, aromatic H), 7.03-7.12 (2H, m, aromatic H), 7.59-7.68 (2H, m, aromatic H).

### EXAMPLE 66

### 8-Cyclopropyl-5-methyl-1-(3-methoxy-4-methyl)phenyl-1,3,4,6-tetrahydro-5H-benz[f]-2,5-oxazocine

8-Bromo-5-methyl-1-(3-methoxy-4-methyl)phenyl-1,3,4,6-tetrahydro-5H-benz[f]-2,5-oxazocine (154 mg, 0.41 mmol), cyclopropylboronic acid (46 mg, 0.53 mmol), P(cyclohexane)₃ (11 mg, 0.041 mmol), K₃PO₄ (304 mg, 1.43 mmol) and Pd(OAc)₂ (5 mg, 0.02 mmol) were heated at 100° C. in toluene (3 ml) and water (150 μl) for 3 hours 15 mins. The reaction mixture was allowed to cool and then diluted with ethyl acetate (30 ml). The organic mixture was washed with water (30 ml) and the aqueous phase extracted into ethyl acetate (3×20 ml). The combined organic extracts were dried (MgSO₄), filtered and concentrated in vacuo to provide the crude product. Purification by silica gel gravity column chromatography [EtOAc] furnished the target product and recovered starting material.

δ_{H} (CDCl₃; 250 MHz) 0.64-0.71 (2H, m, two of cyclopropyl CH₂CH₂), 0.89-0.97 (2H, m, two of cyclopropyl CH₂CH₂), 1.80-1.89 (1H, m, cyclopropyl CH), 2.16 (3H, s, CH₃), 2.48 (3H, s, NCH₃), 2.63 (1H, ddd, J14.2, 5.6, 2.6, one of CH₂CH₂), 2.85 (1H, ddd, J14.2, 8.4, 2.3, one of CH₂CH₂), 3.70 (1H, d, J12.7, one of ArCH₂NR₂), 3.78 (3H, s, OCH₃), 3.84 (1H, ddd, J12.5, 5.6, 2.3, one of CH₂CH₂), 4.17 (1H, ddd, J12.5, 8.4, 2.6, one of CH₂CH₂), 4.70 (1H, d, J12.7, one of ArCH₂NR₂), 5.73 (1H, s, CHOR), 6.69 (1H, dd, J7.6, 1.2, one of ArH), 6.79 (1H, s, ArH), 6.86 (2H, d, J1.2, ArH), 6.94 (1H, s, ArH), 7.03 (1H, d, J7.6, ArH).

### EXAMPLE 67

### 9-Bromo-5-methyl-1-(3,4-methylenedioxy)phenyl-1,3,4,6-tetrahydro-5H-benz[f]-2,5-oxazocine

2-{[2-(Benzo-[1,3]-dioxol-yl-hydroxymethyl)-4-bromobenzyl]-methylamino}-ethanol (148 mg, 0.38 mmol) was heated to reflux temperature with pTSA (107 mg, 0.56 mmol) in toluene (3 ml) under Dean-stark conditions for 3 hours. The mixture was then cooled, diluted with ethyl acetate and washed with sat. aq. sodium bicarbonate solution. The organic phase was dried (MgSO₄), filtered and concentrated in vacuo to provide the crude product. Purification by silica gel gravity column chromatography [3%MeOH/EtOAc to 10%MeOH/EtOAc] furnished product (89 mg, 62%).

δ_{H}(CDCl₃; 250 MHz) 2.43 (3H, s, CH₃), 2.60 (1H, ddd, J14.2, 5.3, 2.9, one of CH₂CH₂), 2.78 (1H, ddd, J14.2, 8.7, 2.4, one of CH₂CH₂), 3.61 (1H, d, J12.7, one of ArCH₂N), 3.81 (1H, ddd, J12.5, 5.3, 2.4, one of CH₂CH₂), 4.20 (1H, ddd, J12.5, 8.7, 2.9, one of CH₂CH₂), 4.84 (1H, d, J12.7, one of ArCH₂N), 5.61 (1H, s, CHOR), 5.93 (2H, s, ROCH₂OR), 6.71-6.75 (3H, m, ArH), 7.07-7.14 (2H, m, ArH), 7.36 (1H, dd, J8.0, 4.0, ArH).

### EXAMPLE 68

### 9-Cyano-5-methyl-1-(3,4-methylenedioxy)phenyl-1,3,4,6-tetrahydro-5H-benz[f]-2,5-oxazocine

A solution of 9-bromo-5-methyl-1-(3,4-methylenedioxy)phenyl-1,3,4,6-tetrahydro-5H-benz[f]-2,5-oxazocine (89 mg, 0.23 mmol), Zn (1 mg, 0.012 mmol), Zn(CN)₂ (16 mg, 0.138 mmol), Zn(OAc)₂ (2 mg, 0.011 mmol), 1,1′-bis(diphenylphosphino)ferrocene (18 mg, 0.05 mmol) and Pd₂(dba)₃ (11 mg, 0.06 mmol) in DMF (2 ml) and H₂O (20 μl) was degassed under vacuum for 15 minutes and then heated at 140° C. overnight. The mixture was then allowed to cool and water added. The organics were extracted into ethyl acetate and then washed with brine to remove the DMF. The combined organic washings were dried (MgSO₄), filtered and concentrated in vacuo to provide the crude product. Purification by silica gel column chromatography [EtOAc] furnished product (21 mg, 27%).

δ_{H} (CDCl₃; 250 MHz) 2.45 (3H, s, NCH₃), 2.63 (1H, ddd, J14.3, 5.2, 2.8, one of CH₂CH₂), 2.76 (1H, ddd, J14.3, 8.4, 2.4, one of CH₂CH₂), 3.70 (1H, d, J12.8, one of ArCH₂NR₂), 3.81 (1H, ddd, J12.6, 5.2, 2.4, one of CH₂CH₂), 4.20 (1H, ddd, J12.6, 8.4, 2.8, one of CH₂CH₂), 4.90 (1H, d, J12.8, one of ArCH₂NR₂), 5.69(1H, s, CHOR), 5.94 (2H, s, ROCH₂OR), 6.69-6.78 (3H, m, ArH), 7.27-7.33 (2H, m, ArH), 7.51 (1H, dd, J7.9, 1.5, ArH).

### EXAMPLE 69

### 9-Cyclopropyl-5-methyl-1-(3,4-methylenedioxy)phenyl-1,3,4,6-tetrahydro-5H-benz[f]-2,5-oxazocine

A mixture of 9-bromo-5-methyl-1-(3,4-methylenedioxy)phenyl-1,3,4,6-tetrahydro-5H-benz[f]-2,5-oxazocine (280 mg, 0.75 mmol), cyclopropylboronic acid (84 mg, 0.97 mmol), K₃PO₄ (555 mg, 2.61 mmol), Pd(OAc)₂ (8.4 mg, 0.04 mmol) and P(cyclohexane)₃ (21 mg, 0.075 mmol) in toluene (5 ml) and H₂O (250 μl) was heated at 100° C. for 3 hours, during which time it turned black. After cooling, water (12 ml) was added and the organics were extracted into ethyl acetate. The combined organic extracts were dried (MgSO₄), filtered and concentrated in vacuo to provide the crude product. Purification (repeated) by silica gel column chromatography [EtOAc] furnished product (56 mg, 22%).

δ_{H} (CDCL₃; 250 MHz) 0.58-0.63 (2H, m, two of cyclopropyl CH₂CH₂), 0.86-0.94 (2H, m, two of cyclopropyl CH₂CH₂), 1.75-1.84 (1H, m, cyclopropyl CH), 2.44 (3H, s, CH₃), 2.55-2.63 (1H, m, one of CH₂CH₂), 2.75-2.84 (1H, m, one of CH₂CH₂), 3.62 (1H, d, J12.5, one of ArCH₂NR₂), 3.81 (1H, ddd, J12.5, 5.6, 2.4, one of CH₂CH₂), 4.14-4.22 (1H, m, one of CH₂CH₂), 4.79 (1H, d, J12.5, one of ArCH₂NR₂), 6.63 (1H, s, CHOR), 5.91 (2H, s, ROCH₂OR), 6.74 and 6.75 (4H, 2 x s, ArH), 6.87 (1H, dd, J7.6, 1.9, ArH), 7.10 (1H, d, J7.9).

### EXAMPLE 70

### 8-Bromo-5-methyl-1-(3,4-methylenedioxy)phenyl-1,3,4,6-tetrahydro-5H-benz[f]-2,5-oxazocine

2-{[2-(Benzo-[1, 3]-dioxol-5-yl-hydroxymethyl)-5-bromobenzyl]-methylamino}-ethanol (715 mg, 1.82 mmol) and pTSA (519 mg, 2.73 mmol) were heated to reflux temperature in toluene (8 ml) under Dean-Stark conditions for 2 hours. Once cooled to RT, the reaction mixture was diluted with ethyl acetate and washed with sat. aq. sodium bicarbonate solution. The organic phases were dried (MgSO₄), filtered and concentrated in vacuo to provide the crude product. Purification by silica gel column chromatography [100% EtOAc to 5%MeOH/EtOAc] yielded 214 mg, (31%).

δ_{H} (CDCl₃; 250 MHz) 2.45 (3H, s, CH₃), 2.61 (1H, ddd, J14.1, 5.8, 2.8, one of CH₂CH₂), 2.79(1H, ddd, J14.1, 8.2, 2.2, one of CH₂CH₂), 3.58 (1H, d, J12.8, one of ArCH₂NR₂), 3.81 (1H, ddd, J12.5, 5.8, 2.2, one of CH₂CH₂), 4.78 (1H, ddd, J12.5, 8.2, 2.8, one of CH₂CH₂), 4.80 (1H, d, J12.8, one of ArCH₂NR₂), 5.64(1H, s, CHOR), 5.91 (2H, s, ROCH₂OR), 6.70-6.77 (3H, m, ArH), 6.87 (1H, d, J8.3, ArH), 7.29-7.37 (2H, m, ArH).

### EXAMPLE 71

### 8-Cyano-5-methyl-1-(3,4-methylenedioxy)phenyl-1,3,4,6-tetrahydro-5H-benz[f]-2,5-oxazocine

8-Bromo-5-methyl-1-(3,4-methylenedioxy)phenyl-1,3,4,6-tetrahydro-5H-benz[f]-2,5-oxazocine (99 mg, 0.26 mmol), Zn (1 mg, 0.015 mmol), Zn(OAc)₂ (6 mg, 0.033 mmol), Zn(CN)₂ (18 mg, 0.156 mmol), 1,1′-bis(diphenylphosphino)ferrocene (18 mg, 0.054 mmol) and Pd₂(dba)₃ (33 mg, 0.036 mmol) were degassed and then heated at 140° C. overnight. The reaction mixture was cooled and water added. The organics were extracted three times with ethyl acetate, and the combined organic extracts were washed with brine, dried (MgSO₄), filtered and concentrated in vacuo to provide the crude target as a black oil. Purification by silica gel column chromatography [EtOAc] furnished product 21 mg, (25%).

δ_{H} (CDCl₃; 250 MHz) 2.46 (3H, s, CH₃), 2.63 (1H, ddd, J14.3, 5.4, 3.1, one of CH₂CH₂), 2.75(1H, ddd, J14.3, 8.3, 2.6, one of CH₂CH₂), 3.64(1H, d, J12.8, one of ArCH₂NR₂), 3.81 (1H, ddd, J12.7, 5.4, 2.6, one of CH₂CH₂), 4.24 (1H, ddd, J12.7, 8.3, 3.1, one of CH₂CH₂), 4.97 (1H, d, J12.8, one of ArCH₂NR₂), 5.68 (1H, CHOR), 5.93 (2H, s, ROCH₂OR), 6.69-6.78 (3H, m, ArH), 7.12 (1H, d, J7.9, ArH), 7.45-7.52 (2H, m, ArH).

### EXAMPLE 72

### 8-Cyclopropyl-5-methyl-1-(3,4-methylenedioxy)phenyl-1,3,4,6-tetrahydro-5H-benz[f]-2,5-oxazocine

8-Bromo-5-methyl-1-(3,4-methylenedioxy)phenyl-1,3,4,6-tetrahydro-5H-benz[f]-2,5-oxazocine (90 mg, 0.24 mmol), cyclopropylboronic acid (26.8 mg, 0.312 mmol), K₃PO₄ (178 mg, 0.84 mmol), Pd(OAc)₂ (3 mg, 0.012 mmol) and P(cyclohexane)₃ (7 mg, 0.024 mmol) were heated to 100° C. in toluene (2 ml) and water (100 μl) for 3 hours. The reaction mixture was cooled, diluted with water and extracted with ethyl acetate. The combined organic extracts were dried (MgSO₄), filtered and concentrated in vacuo to provide the crude product. Purification was by silica gel column chromatography [100%EtOAc to 5%MeOH/EtOAc] furnished 8-cyclopropyl-(3,4-methylenedioxy)-nefopam 49 mg,(61%).

δ_{H} (CDCl₃; 250 MHz) 0.67-0.72 (2H, m, two of cyclopropyl CH₂CH₂), 0.91-0.99 (2H, m, two of cyclopropyl CH₂CH₂), 2.48 (3H, s, CH₃), 2.61 (1H, ddd, J14.2, 5.7, 2.6, one of CH₂CH₂), 2.83 (1H, ddd, J14.2, 8.4, 2.5, one of CH₂CH₂), 3.64 (1H, d, J12.8, one of ArCH₂NR₂), 3.83 (1H, ddd, J12.5, 5.7, 2.5, one of CH₂CH₂), 4.42 (1H, ddd, J12.5, 8.4, 2.6, one of CH₂CH₂), 4.74 (1H, d, J12.8, one of ArCH₂NR₂), 5.67 (1H, s, CHOH), 5.91 (2H, s, ROCH₂OR), 6.75 (3H, s, ArH), 6.88-6.9 (3H, m, ArH)

### EXAMPLE 73

### 8-Bromo-5-methyl-1-(3-ethoxy)phenyl-1,3,4,6-tetrahydro-5H-benz[f]-2,5-oxazocine

2-({5-Bromo-2-[hydroxyl-(3-ethoxy-phenyl)-methyl]-benzyl}-methylamino)-ethanol (1.06 g, 2.69 mmol) was heated to reflux temperature under Dean-Stark conditions with pTSA (0.78 g, 4.03 mmol) in toluene (40 ml) for 2.5 h. The reaction mixture was allowed to cool and then diluted with ethyl acetate (20 ml). The mixture was washed with sat. aq sodium bicarbonate solution (2×20 ml) and the combined organic extracts were dried (MgSO₄), filtered and concentrated in vacuo to provide 0.94 g of crude product. Purification by silica gel gravity column chromatography [EtOAc] gave the product (0.60 g, 59%) as a colourless oil. 8H (CDCl₃; 250 MHz) 7.37-6.87 (7H, m, aromatics), 5.71 (1H, s, CH-O), 4.77 (1H, d, J 13, ArCH_{A}H_{B}N), 4.14 (1H, ddd, J 13, 8 and 2.5, CH_{A}H_{B}CH₂), 4.00 (2H, q, J 7, CH₂CH₃), 3.84 (1H, ddd, J 13, 8 and 2.5, CH_{A}H_{B}CH₂), 3.62 (1H, d, J 13, ArCH_{A}H_{B}N), 2.85-2.79 (1H, m, CH_{A}H_{B}CH₂), 2.68-2.60 (1H, m, CH_{A}H_{B}CH₂), 2.46 (3H, s, NCH₃), 1.39 (3H, t, J 7, CH₂CH₃).

### EXAMPLE 74

### 8-Cyano-5-methyl-1-(3-ethoxy)phenyl-1,3,4,6-tetrahydro-5H-benz[f]-2,5-oxazocine

8-Bromo-5-methyl-1-(3ethoxy)phenyl-1,3,4,6-tetrahydro-5H-benz[f]-2,5-oxazocine (250 mg, 0.65 mmol), Zn (3 mg, 0.05 mmol), Zn(OAc)₂ (15 mg, 0.077 mmol), Zn(CN)₂ (46 mg, 0.4 mmol), Pd₂(dba)₃ (93 mg, 0.1 mmol) and 1,1′-bis(diphenylphosphino)ferrocene (43 mg, 0.077 mmol) were heated to 140° C. in DMF (5 ml) and H₂O (100 μl) for 4 h. The black reaction mixture was allowed to cool to RT and diluted with water. The resulting brown precipitate was collected by filtration and washed with water then brine. The filtrate was extracted with ethyl acetate (2×60 ml). The combined organic extracts were dried (MgSO₄), filtered and concentrated in vacuo to provide the crude product as a brown oil. Purification by silica gel gravity column chromatography [DCM] gave clean product (56 mg, 27%).

δ_{H}(CDCl₃; 250 MHz) 7.50-6.78 (7H, m, aromatics), 5.72 (1H, s, CHO), 4.96 (1H, d, J 13, ArCH_{A}H_{B}—N), 4.39-4.19 (1H, m, CH_{A}H_{B}CH₂), 3.98 (2H, q, J7, CH₂CH₃), 3.85-3.79 (1H, m, CH_{A}H_{B}CH₂), 3.67 (1H, d, J 13, ArCH_{A}H_{B}—N), 2.82-2.58 (2H, m, CH₂CH₂), 2.43 (3H, s, NCH₃), 1.38 (3H, t, J7, CH₂CH₃).

### EXAMPLE 75

### 8-Cyclopropyl-5-methyl-1-(3-ethoxy)phenyl-1,3,4,6-tetrahydro-5H-benz[f]-2,5-oxazocine

8-Bromo-5-methyl-1-3-ethoxy)phenyl-1,3,4,6-tetrahydro-5H-benz[f]-2,5-oxazocine (100 mg, 0.26 mmol), cyclopropylboronic acid (29 mg, 0.33 mmol), K₃PO₄ (193 mg, 0.91 mmol), Pd(OAc)₂(3 mg, 0.013 mmol) and P(cyclohexane)₃ (8 mg, 0.026 mmol) were heated to 100° C. in toluene (2 ml) and H₂O (100 μl) for 4 hours. After cooling to RT, water (2 ml) was added and the mixture extracted with ethyl acetate, washed with brine, dried (MgSO₄), filtered and concentrated in vacuo. Purification by silica gel gravity column chromatography [DCM] furnished clean product (20 mg, 23%) as a colourless oil.

δ_{H}(CDCl₃; 250 MHz) 7.26-6.76 (7H, m, aromatics), 5.71 (1H, s, CHO), 4.75 (1H, d, J 12.5, ArCH_{A}H_{B}—N) 4.22-4.12 (1H, m, CH_{A}H_{B}CH₂), 3.99 (2H, q, J7, CH₂CH₃), 3.88-3.79 (1H, m, CH_{A}H_{B}CH₂), 3.81 (1H, d, J 12.5, ArCH_{AH}_{B}—N), 2.90-2.61 (2H, m, CH₂CH₂), 2.50 (3H, s, NCH₃), 1.89-1.82 (1H, m, CHCH₂), 1.39 (3H, t, J7, CH₂CH₃), 0.96-0.92 (2H, m, CH₂CH₂), 0.70-0.67 (2H, m, CH₂CH₂).

### EXAMPLE 76

### 9-Bromo-5-methyl-1-(3-ethoxy)phenyl-1,3,4,6-tetrahydro-5H-benz[f]-2,5-oxazocine

2-({4-Bromo-2-[hydroxyl-(3ethoxy-phenyl)-methyl]-benzyl}-methylamino)-ethanol (1.20 g, 3.04 mmol) was heated to reflux temperature under Dean-Stark conditions with pTSA (0.88 g, 4.57 mmol) in toluene (50 ml) for 4 h. The reaction mixture was allowed to cool and then diluted with ethyl acetate (25 ml). The mixture was washed with sat. aq sodium bicarbonate solution (2×25 ml), and the combined organic extracts were dried (MgSO₄), filtered and concentrated in vacuo to provide 1.4 g of crude product. Purification by silica gel gravity column chromatography [EtOAc] gave product (0.59 g, 51%) as a colourless oil.

δ_{H} (CDCl₃; 250 MHz) 7.37-6.76 (7H, m, aromatics), 5.67 (1H, s, CH—O), 4.79 (1H, d, J 13, ArCH_{A}H_{B}N), 4.16 (1H, ddd, J 13, 8 and 2.5, CH_{A}H_{B}CH₂), 4.01 (2H, q, J 7, CH₂CH₃), 3.81 (1H, ddd, J 13, 8 and 2.5, CH_{A}H_{B}CH₂), 3.63 (1H, d, J 13, ArCH_{A}H_{B}N), 2.84-2.78 (1H, m, CH_{A}H_{B}CH₂), 2.66-2.63 (1H, m, CH_{A}H_{B}CH₂), 2.44 (3H, s, NCH₃), 1.40 (3H, t, J7, CH₂CH₃).

### EXAMPLE 77

### 9-Cyano-5-methyl-1-(3-ethoxy)phenyl-1,3,4,6-tetrahydro-5H-benz[f]-2,5-oxazocine

9-Bromo-5-methyl-1-(3-ethoxy)phenyl-1,3,4,6-tetrahydro-5H-benz[f]-2,5-oxazocine (150 mg, 0.39 mmol), Zn (2 mg, 0.03 mmol), Zn(OAc)₂ (9 mg, 0.046 mmol), Zn(CN)₂ (28 mg, 0.24 mmol), Pd₂(dba)₃ (56 mg, 0.06 mmol) and 1,1′-bis(diphenylphosphino)ferrocene (25 mg, 0.046 mmol) were heated to 140° C. in DMF (3 ml) and H₂O (30 μl) overnight. The black reaction mixture was allowed to cool to RT and diluted with water. The resulting brown precipitate was collected by filtration and washed with water then brine. The filtrate was extracted with ethyl acetate (2×80 ml), and the combined organic extracts dried (MgSO₄), filtered and concentrated in vacuo to provide the crude product. Purification by silica gel gravity column chromatography [DCM] gave clean product (12 mg, 10%). δ_{H}(CDCl₃; 250 MHz) 7.53-6.80 (7H, m, aromatics), 5.75 (1H, s, CHO), 4.87 (1H, d, J 13, ArCH_{A}H_{B}N), 4.29-4.14 (1H, m, CH_{A}H_{B}CH_{z}), 4.01 (2H, q, J7, CH₂CH₃), 3.88-3.78 (1H, m, CH_{A}H_{B}CH₂), 3.73 (1H, d, J 13, ArCH_{A}H_{B}N), 2.82-2.71 (1H, m, CH_{A}H_{B}—CH₂), 2.68-2.59 (1H, m, CH_{A}H_{B}CH₂), 2.46 (3H, s, N—CH₃), 1.39 (3H, t, J7, CH₂CH₃).

### EXAMPLE 78

### 9-Cyclopropyl-5-methyl-1-(3-ethoxy)phenyl-1,3,4,6-tetrahydro-5H-benz[f]-2,5-oxazocine

9-Bromo-5-methyl-1-(3-ethoxy)phenyl-1,3,4,6-tetrahydro-5H-benz[f]-2,5-oxazocine (140 mg, 0.36 mmol), cyclopropylboronic acid (41 mg,0.45 mmol), K₃PO₄ (269 mg, 1.26 mmol), Pd(OAc)₂ (5 mg, 0.02 mmol) and P(cyclohexane)₃ (11 mg, 0.04 mmol) were heated to 100° C. in toluene (3 ml) and H₂O (150 μl) for 4 hours. After cooling to RT, water (2 ml) was added and the mixture extracted with ethyl acetate, washed with brine, dried (MgSO₄), filtered and concentrated in vacuo. Purification by silica gel gravity column chromatography [DCM] gave product (61 mg, 50%) as a colourless oil.

δ_{H}(CDCl₃; 250 MHz) 7.25-6.76 (7H, m, aromatics), 5.69 (1H, s, CHO), 4.79 (1H, d, J 13, ArCH_{A}H_{B}N), 4.23-4.16 (1H, m, CH_{A}H_{B}CH₂), 4.01 (2H, q, J7, CH₂CH₃), 3.89-3.81 (1H, m, CH_{A}H_{B}CH₂), 3.70 (1H, d, J 13, ArCH_{A}H_{B}N), 2.88-2.78 (1H, m, CH_{A}H_{B}—CH₂),2.68-2.60 (1H, m, CH_{A}H_{B}CH₂), 2.48 (3H, s, N—CH₃), 1.83-1.72 (1H, m, CHCH₂), 1.39 (3H, t, J 7, CH₂CH₃), 0.92-0.88 (2H, m, CH₂CH₂), 0.62-0.58 (2H, m, CH₂CH₂).

### EXAMPLE 79

### 8-Bromo-5-methyl-1-(3-trifluoromethoxy)phenyl-1,3,4,6-tetrahydro-5H-benz[f]-2,5-oxazocine

2-({5-bromo-2-[hydroxyl-(3-trifluoromethoxy-phenyl)-methyl]-benzyl}-methylamino)-ethanol (635 mg, 1.47 mmol) was heated to reflux under Dean-Stark conditions with pTSA (420 mg, 2.21 mmol) in toluene (6 ml) for 2 hours. The reaction mixture was allowed to cool to RT and then diluted with ethyl acetate (30 ml). The mixture was washed with sat. aq. sodium bicarbonate solution (50 ml) and was extracted into ethyl acetate. The combined organic extracts were dried (MgSO₄), filtered and concentrated in vacuo to provide the crude cyclised product. Purification by silica gel gravity column chromatography [EtOAc] furnished the target product (116 mg, 19%).

δ_{H} (CDCl₃; 250 MHz) 2.47 (3H, s, CH₃), 2.67 (1H, ddd, J14.3, 6.1, 2.5, one of CH₂CH₂), 2.84 (1H, ddd, J14.3, 7.8, 2.2, one of CH₂CH₂), 3.67 (1H, d, J12.8, one of ArCH₂NR₂), 3.86 (1H, ddd, J12.8, 6.1, 2.2, one of CH₂CH₂), 4.13 (1H, ddd, J12.8, 7.8, 2.5, one of CH₂CH₂), 4.61 (1H, d, J12.8, one of ArCH₂NR₂), 5.79 (1H, s, CHOR), 6.85 (1H, d, J8.2, ArH), 7.11-7.19 (3H, m, ArH), 7.31-7.36 (3H, m, ArH).

### EXAMPLE 80

### 8-Cyano-5-methyl-1-(3-trifluoromethoxy)phenyl-i,3,4,6-tetrahydro-5H-benz[f]-2,5-oxazocine

8-Bromo-5-methyl-1-(3-trifluoromethoxy)phenyl-1,3,4,6-tetrahydro-5H-benz[f]-2,5-oxazocine (116 mg, 0.28 mmol), Zn (1 mg, 0.02 mmol), Zn(OAc)₂ (7 mg, 0.038 mmol), Zn(CN)₂ (20 mg, 0.17 mmol), Pd₂(dba)₃ (40 mg, 0.04 mmol) and 1,1′-bis-(diphenylphosphine)ferrocene (20 mg, 0.036 mmol) were heated to 140° C. in DMF (3 ml) and H₂O (30 μl) overnight. The black reaction mixture was allowed to cool and diluted with water. The resulting brown precipitate was collected by filtration and washed with ethyl acetate. The filtrate was washed with brine (2×60 ml) and the combined aqueous phases were then washed with ethyl acetate (2×80 ml). The combined organic extracts were dried (MgSO₄) and concentrated in vacuo to provide the crude product. Purification by silica gel gravity column chromatography [1:1 ethyl acetate/heptane) furnished the desired product (9 mg, 9%).

δ_{H}(CDCl₃; 250 MHz) 2.50 (3H,s, CH₃), 2.61-2.87 (2H, m, two of CH₂CH₂), 3.76 (1H, d, J13.1, one of ArCH₂NR₂), 3.87 (1H, ddd, J12.7, 5.4, 2.5, one of CH₂CH₂), 4.25 (1H, ddd, J12.7, 8.4, 3.1, one of CH₂CH₂), 4.86 (1H, d, J13.1, one of ArCHNR₂), 5.82 (1H, s, CHOR), 7.11-7.16 (4H, m, ArH), 7.33-7.39 (1H, m, ArH), 7.52 (1H, dd, J7.9, 1.8, ArH), 7.57 (1H, bs, ArH).

### EXAMPLE 81

### 8-Cyclopropyl -5-methyl-1-(3-trifluoromethoxy)phenyl-1,3,4,6-tetrahydro-5H-benz[f]-2,5-oxazocine

8-Bromo-5-methyl-1-(3-trifluoromethoxy)phenyl-1,3,4,6-tetrahydro-5H-benz[f]-2,5-oxazocine (160 mg, 0.39 mmol), cyclopropylboronic acid (43 mg,0.5 mmol), K₃PO₄ (288 mg, 1.36 mmol), Pd(OAc)₂ (4 mg, 0.019 mmol) and P(cyclohexane)₃ (11 mg, 0.039 mmol) were heated to 100° C. in toluene (2 ml) and H₂O (100 μl) for 4 hours. The reaction mixture, now black in colour, was allowed to cool to RT. Water (50 ml) was added and the mixture extracted into ethyl acetate (3×80 ml). The combined organic extracts were dried (MgSO₄), filtered and concentrated in vacuo to provide the crude cyclopropyl analogue. Purification by silca gel column chromatography [1:1 EtOAc/heptane, then 100% EtOAc] furnished 8-cyclopropyl-(m-trifluoromethoxy)-nefopam.

δ_{H} (CDCl₃; 250 MHz) 0.67-0.74 (2H, m, two of cyclopropyl CH₂CH₂), 0.89-1.00 (2H, m, two of cyclopropyl CH₂CH₂), 1.82-1.93 (1H, m, cyclopropyl CH), 2.51 (3H, s, CH₃), 2.68 (1H, ddd, J14.2, 5.7, 2.5, one of CH₂CH₂), 2.89 (1H, ddd, J14.2, 8.2, 2.0, one of CH₂CH₂), 3.74 (1H, d, J12.8, one of ArCH₂NR₂), 3.87 (1H, ddd, J12.7, 5.7, 2.0, one of CH₂CH₂), 4.13 (1H, ddd, J12.7, 8.2, 2.5, one of CH₂CH₂), 4.58 (1H, d, J12.5, one of ArCH₂NR₂), 5.81 (1H, s, CHOR), 6.84-6.97 (3H, m, ArH), 7.09-7.20 (3H, m, ArH), 7.29-7.37 (1H, m, ArH).

### EXAMPLE 82

### 9-Bromo-5-methyl-1-(3-trifluoromethoxy)phenyl-1,3,4,6-tetrahydro-5H-benz[f]-2,5-oxazocine

Crude 2-({4-bromo-2-[hydroxyl-(3-trifluoromethoxy-phenyl)-methyl]-benzyl}-methylamino)-ethanol (1.159 g, 2.69 mmol) was heated to reflux with pTSA (768 mg, 4.04 mmol) in xylene (12 ml) with Dean-Stark for 4 hours, 45 mins. The reaction mixture was allowed to cool and then washed with a saturated solution of sodium bicarbonate (100 ml). The organics were extracted into ethyl acetate (2×100 ml) and the combined organic extracts dried (MgSO₄), filtered and concentrated in vacuo to provide the crude product as a 2:1 mixture with starting material. Purification by silica gel column chromatography provided the target product (189 mg, 17%).

δ_{H} (CDCl₃; 250 MHz) 2.44 (3H, s, CH₃), 2.64 (1H, ddd, J15.0, 5.0, 2.5, one of CH₂CH₂), 2.81 (1H, ddd, J15.0,7.5,2.5, one of CH₂CH₂), 3.67 (1H, d, J12.5, one of ArCH₂NR₂), 3.84 (1H, ddd, J12.5, 5.0, 2.5, one of CH₂CH₂), 4.14 (1H, ddd, J12.5, 5.0, 2.5, one of CH₂CH₂), 4.65 (1H, d, J12.5, one of ArCH₂NR₂), 5.75 (1H, S, CHOR), 7.09-7.19 (5H, m, ArH), 7.34 (1H, d, J8.0, one of ArH), 7.40 (1H, dd, J8.0, 1.9, one of ArH).

### EXAMPLE 83

### 9-Cyclopropyl-5-methyl-1-(3-trifluoromethoxy)phenyl-1,3,4,6-tetrahydro-5H-benz[f]-2,5-oxazocine

9-Bromo-5-methyl-1-(3-trifluoromethoxy)phenyl-1,3,4,6-tetrahydro-5H-benz[f]-2,5-oxazocine (95 mg, 0.28 mmol), K₃PO₄ (205 mg, 0.97 mmol), cyclopropylboronic acid (31 mg, 0.36 mmol), P(cyclohexane)₃ (8 mg, 0.028 mmol) and Pd(OAc)₂ (3 mg, 0.014 mmol) were heated at 100° C. in toluene (2 ml) and water (100 μl). The reaction mixture turned black in colour after about 20 mins. At the end of 3 hours, the mixture was allowed to cool to RT, and water (30 ml) was added. The organics were extracted into ethyl acetate (2×30 ml) and the combined extracts dried (MgSO₄), filtered and concentrated in vacuo. Purification by silica gel gravity column chromatography [EtOAc] furnished product.

δ_{H} (CDCl₃; 250 MHz) 0.58-4.63 (2H, m, two of cyclopropyl CH₂CH₂), 0.90-0.94 (2H, m, two of CH₂CH₂), 1.73-1.79 (1H, m, cyclopropyl CH), 2.45 (3H, s, CH₃), 2.68 (1H, ddd, J15.0, 5.0, 2.5, one of CH₂CH₂), 2.87 (1H, ddd, J15.0, 7.5, 2.5, one of CH₂CH₂), 3.75 (1H, d, J12.5, one of ArCH₂NR₂), 3.88 (1H, ddd, J 12.5, 5.0, 2.5, one of CH₂CH₂), 4.19 (1H, ddd, J 12.5, 7.5, 2.5, one of CH₂CH₂), 4.67 (1H, J12.5, one of ArCH₂NR₂), 5.75 (1H, s, CHOR), 6.72 (1H, d, J2.5, one of ArH), 6.93 (1H, dd, J7.5, 2.5, ArH), 7.10-7.19 (4H, m, ArH), 7.29-7.38 (1H, ArH)

### EXAMPLE 84

### 9-Bromo-5-methyl-1-(2-methyl)phenyl-1,3,4,6-tetrahydro-5H-benz[f]-2,5-oxazocine

2-[(4-bromo-2-(hydroxyl-o-tolylmethyl)-benzyl)-methylamino]-ethanol (790 mg, 2.18 mmol) was dissolved in toluene (8 ml) at RT. pTSA (623 mg, 3.27 mmol) was added and the resulting mixture was heated to reflux temperature under Dean-Stark conditions for 1.5 hours. Once cooled to RT, the mixture was diluted with ethyl acetate and washed with sat. aq. solution of sodium bicarbonate. The organic extracts were dried (MgSO₄), filtered and concentrated in vacuo to provide the crude material as an oil Purification by silica gel gravity column chromatography [20%EtOAc/heptane to 30%EtOAc] furnished the product (357 mg, 55% corrected yield).

δ_{H} (CDCl₃; 250 MHz) 2.31 (3H, s, ArCH₃), 2.44 (3H, s, NCH₃), 2.63 (1H, ddd, J14.4, 5.3,2.4, one of CH₂CH₂), 2.86 (1H, ddd, J14.4, 8.9, 2.1, one of CH₂CH₂), 3.76 (1H, d, J, 13.0, one of ArCH₂NR₂), 3.85 (1H, ddd, J12.7, 5.3, 2.1, one of CH₂CH₂), 4.12(1H, ddd, J12.7, 8.9, 2.4, one of CH₂CH₂), 4.68(1H, d, J13.0, one of ArCH₂NR₂), 5.95 (1H, s, CHOR), 7.02-7.22 (6H, m, ArH), 7.38 (1H, dd, J8.2, 2.1, ArH).

### EXAMPLE 85

### 9-Cyano-5-methyl-1-(2-methyl)phenyl-1,3,4,6-tetrahydro-5H-benz[f]-2,5-oxazocine

9-Bromo-5-methyl-1-(2-methyl)phenyl-1,3,4,6-tetrahydro-5H-benz[f]-2,5-oxazocine (156 mg, 0.45 mmol), Zn dust (2 mg, 0.03 mmol), Zn(CN)₂ (32 mg, 0.27 mmol), Zn(OAc)₂ (10 mg, 0.054 mmol), 1,1′-bis(diphenylphosphino)ferrocene (30 mg, 0.054 mmol) and Pd₂(dba)₃ (55 mg, 0.06 mmol) were heated to 140° C. in DMF (3 ml) and water (30 μl) under a nitrogen atmosphere over-night. After cooling, water was added and the resulting black/brown precipitate was collected by suction filtration and washed well with ethyl acetate. The filtrate was washed with brine and extracted into ethyl acetate. The combined organic extracts were dried (MgSO₄), filtered and concentrated in vacuo to provide the crude material as a very dark brown oil. Purification by silica gel gravity column chromatography [EtOAc] furnished the product (79 mg, 60%).

δ_{H} (CDCl₃; 250 MHz) 2.28 (3H, s, ArCH₃), 2.45 (3H, s, ArCH₃), 2.65 (1H, ddd, J14.4, 5.5, 2.4, one of CH₂CH₂), 2.85 (3H, ddd, J14.4, 8.9, 2.1, one of CH₂CH₂), 3.82-3.90 (2H, m, one of CH₂CH₂ and one of ArCH₂NR₂), 4.07-4.17 (1H, m, one of CH₂CH₂), 4.74 (1H, d, J12.8, one of ArCH₂NR₂), 6.02 (1H, s, CHOR), 7.05 (1H, d, J6.7, ArH), 7.14-7.27 (4H, m, ArH), 7.34(1H, d, J7.9, ArH), 7.54 (1H, dd, J7.9, 1.7, ArH).

### EXAMPLE 86

### 9-Cyclopropyl-5-methyl-1-(2-methyl)phenyl-1,3,4,6-tetrahydro-5H-benz[f]-2,5-oxazocine

9-Bromo-5-methyl-1-(2-methyl)phenyl-1,3,4,6-tetrahydro-5H-benz[f]-2,5-oxazocine (188 mg, 0.55 mmol) was dissolved in toluene (4 ml) and water (200 μl) at RT. Cyclopropylboronic acid (61 mg, 0.71 mmol), K₃PO₄ (409 mg, 1.93 mmol), Pd(OAc)₂(6 mg, 0.03 mmol) and P(cyclohexane)₃ (15.4 mg, 0.055 mmol) were added. The mixture was heated to 100° C. for a total of 3 hours, during which time the reaction mixture became black. After cooling to RT, the mixture was diluted with water and extracted into ethyl acetate. The organic phases were dried (MgSO₄), filtered and concentrated in vacuo to provide the crude product. Purification by silica gel gravity column chromatography [EtOAc] furnished the product (129 g, 81%).

δ_{H} (CDCl₃; 250 MHz) 0.54-0.60 (2H, m, two of cyclopropyl CH₂CH₂), 0.86-0.89 (2H, m, two of cyclopropyl CH₂CH₂), 1.69-1.80 (1H, m, cyclpropyl CH), 2.34 (3H, s, ArCH₃), 2.45 (3H, s, ArCH₃), 2.62 (1H, ddd, J14.3, 5.5, 2.4, one of CH₂CH₂), 2.87 (1H, ddd, J14.3, 8.5, 2.1, one of CH₂CH₂), 3.75 (1H, d, J13.0, one of ArCH₂NR₂), 3.85 (1H, ddd, J12.7, 5.5, 2.1, one of CH₂CH₂), 4.11 (1H, ddd, J12.7, 8.5, 2.4, one of CH₂CH₂), 4.64 (1H, d, J13.0, one of ArCH₂NR₂), 5.96 (1H, s, CHOR), 6.62 (1H, d, J1.8, ArH), 6.89 (1H, dd, J7.9, 1.8, ArH), 7.05-7.20 (5H, ArH).

### EXAMPLE 87

### 8-Bromo-5-methyl-1-(2-methyl)phenyl-1,3,4,6-tetrahydro-5H-benz[f]-2,5-oxazocine

2[(5-bromo-2-(hydroxyl-o-tolylmethyl)-benzyl)methylamino]-ethanol (777 mg, 2.16 mmol) was dissolved in toluene (8 ml) at RT. pTSA (616 mg, 3.24 mmol) was added, and the resulting mixture heated at reflux temperature under Dean-Stark conditions for 4 hours. The reaction was allowed to cool to RT and then diluted with ethyl acetate, and washed with sat. aq. sodium bicarbonate solution. The organic washings were dried (MgSO₄), filtered and concentrated in vacuo to provide the impure product. Purification by silca gel gravity column chromatography [1:1 EtOAc/heptane to 100% EtOAc] gave the desired product (504 mg, 64%).

δ_{H} (CDCl₃; 250 MHz) 2.29 (3H, s, ArCH₃), 2.46 (3H, s, NCH₃), 2.66 (1H, ddd, J14.4, 5.6, 2.4, one of CH₂CH₂), 2.89 (1H, ddd, J14.4, 8.5, 2.1, one of CH₂CH₂), 3.77 (1H, d, J13.1, one of ArCH₂NR₂), 3.86 (1H, ddd, J12.7, 5.6, 2.1, one of CH₂CH₂), 4.09 (1H, ddd, J12.7, 8.5, 2.4, one of CH₂CH₂), 4.64 (1H, d, J13.1, one of ArCH₂NR₂), 5.97 (1H, s, CHOR), 6.74 (1H, d, J8.2, ArH), 7.07-7.20 (4H, m, ArH), 7.29 (1H, dd, J8.6, 2.1, ArH), 7.41 (1H, d, J2.1, ArH).

### EXAMPLE 88

### 8-Cyano-5-methyl-1-(2-methyl)phenyl-1,3,4,6-tetrahydro-5H-benz[f]-2.5-oxazocine

8-Bromo-5-methyl-1-(2-methyl)phenyl-1,3,4,6-tetrahydro-5H-benz[f]-2,5-oxazocine (207 mg, 0.60 mmol), Zn (2 mg,0.03 mmol), Zn(OAc)₂ (13 mg, 0.071 mmol), Zn(CN)₂ (42 mg, 0.36 mmol), 1,1′-bis(diphenylphosphino)ferrocene (40 mg, 0.072 mmol) and Pd₂(dba)₃ (75 mg, 0.082 mmol) were heated to 140° C. In DMF (5 ml) and H₂O (50 μl) overnight. The black reaction mixture was cooled and water added, producing a brown precipitate. The precipitate was washed with ethyl acetate and the filtrate washed with brine to remove the DMF. The organic phase was dried (MgSO₄), filtered and concentrated in vacuo to provide the crude product. Purification by silica gel gravity column chromatography [EtOAc] furnished the desired product (39 g, 22%).

δ_{H} (CDCl₃; 250 MHz) 2.33 (3H, s, ArCH₃), 2.46 (3H, s, NCH₃), 2.64 (1H, ddd, J14.9, 5.1, 2.6, one of CH₂CH₂), 2.77 (1H, ddd, J14.9, 8.9, 2.4, one of CH>₂CH₂), 3.78 (1H, d, J13.1, one of ArCH₂NR₂), 3.86 (1H, ddd, J12.7, 5.1, 2.4, one of CH₂CH₂), 4.18(1H, ddd, J12.7, 8.9, 2.6, one of CH₂CH₂), 4.89(1H, d, J13.1, one of ArCH₂NR₂), 5.99 (1H, s, CHOR), 6.91-6.98 (2H, m, ArH), 7.12-7.22 (3H, ArH), 7.46 (1H, dd, J7.9, 1.8, ArH), 7.55 (1H, d, J1.5, ArH).

### EXAMPLE 89

### 8-Cyclopropyl -5-methyl-1-(2-methyl)phenyl-1,3,4,6-tetrahydro-5H-benz[f]-2,5-oxazocine

8-Bromo-5-methyl-1-(2-methyl)phenyl-1,3,4,6-tetrahydro-5H-benz[f]-2,5-oxazocine (205 mg, 0.60 mmol) was dissolved in toluene (4 ml) and H₂O (200 μl) at RT. Cyclopropylboronic acid (67 mg, 0.77 mmol), K₃PO₄ (446 mg, 2.1 mmol), Pd (OAc)₂ (7 mg, 0.03 mmol) and P(cyclohexane)₃ (17 mg, 0.06 mmol) were added and the resulting mixture heated at 100° C. for 3 hours. The mixture was then allowed to cool to RT before partitioning the mixture between water and ethyl acetate. The organic phases were dried (MgSO₄), filtered and concentrated in vacuo to yield the crude product. Purification by silica gel gravity column chromatography [EtOAc] provided the desired target (117 g, 10 64%).

δ_{H}(CDCl₃; 250 MHz) 0.67-0.74 (2H, m, two of CH₂CH₂), 0.92-1.0 (2H, m, two of CH₂CH₂), 1.83-1.93 (1H, m, cyclopropyl CH), 2.28 (3H, s, ArCH₃), 2.48 (3H, s, NCH₃), 2.66 (1H, ddd, J14.4, 5.6, 2.3, one of CH₂CH₂), 2.95 (1H, ddd, J14.4, 8.5, 2.1, one of CH₂CH₂), 3.81 (1H, d, J12.8, one of ArCH₂), 3.87 (1H, ddd, J12.8, 5.6, 2.1, one of CH₂CH₂), 4.08 (1H, ddd, J12.8, 8.5, 2.3, one of CH₂CH₂), 4.56 (1H, d, J12.8, one of ArCH₂NR₂), 5.98 (1H, s, CHOR), 6.73 (1H, d, J7.9, ArH), 6.85 (1H, dd, J7.9, 1.8, ArH), 6.97 (1H, d, J1.8, ArH), 7.14-7.18 (4H, m, ArH).

### EXAMPLE 90

### 9-Bromo-5-methyl-1-(3-pyridyl)-1,3,4,6-tetrahydro-5H-benz[f]-2,5-oxazocine

2-{[4-Bromo-2-(hydroxypyridin-3-yl-methyl)-benzyl]-methylamino}-ethanol (100 mg, 0.29 mmol) was dissolved in xylene (2 mL) at RT. pTSA (434 mg, 2.28 mmol) was added, and the resulting mixture was heated at 120° C. in an open vessel until all solvent and water had evaporated. The reaction vessel was removed from the heat and allowed to cool. Ethyl acetate (20 ml) and a sat. aq. solution of sodium bicarbonate (20 ml) were added to partition the gummy mixture. The organic phase was separated, and the aqueous phase was extracted into ethyl acetate (2×20 ml). Combined extracts were dried (MgSO₄), filtered and concentrated in vacuo to give the cyclised product as a pale brown oil. Purification by silica gel column chromatography with a stepped gradient, [100% EtOAc to 3%/5%/10% MeOH/EtOAc)], furnished product (23 mg, 24%).

δ_{H}(CDCl₃, 250 MHz) 2.46 (3H, s, NCH₃), 2.66 (1H, ddd, J14.4, 5.5, 2.8, one of CH₂CH₂), 2.83 (1H, ddd, J14.4, 8.2, 2.1, one of CH₂CH₂), 3.67 (1H, d, J13.1, one of ArCH₂NR₂), 3.86 (1H, ddd, J12.7, 5.5, 2.1, one of CH₂CH₂), 4.19 (1H, ddd, J12.7, 8.2, 2.8, one of CH₂CH₂), 4.73 (1H, d, J13.1, one of ArCH₂NR₂), 5.77(1H, s, CHOR), 7.11-7.14 (2H, m, aromatic H), 7.24-7.29 (1H, m, aromatic H), 7.39 (1H, dd, J7.9, 1.8, aromatic H), 7.56 (1H, d, J7.9, aromatic H), 8.53-8.57 (2H, m, aromatic H).

### EXAMPLE 91

### 8-Bromo-5-methyl-1-(3-pyridyl)-1,3,4,6-tetrahydro-5H-benz[f]-2,5-oxazocine

2-{[5-Bromo-2-hydroxypyridin-3-yl-methyl)-benzyl]-methylamino}-ethanol (84 mg, 0.24 mmol) was dissolved in xylene (2 ml). pTSA (364 mg, 1.9 mmol) was added and the mixture was heated to reflux temperature in an open vessel to allow evaporation of the solvent and water. When the mixture had concentrated down to a dark gum, the vessel was removed from the heat and allowed to cool. The residue was partitioned between ethyl acetate (20 ml) and sat. eq. sodium bicarbonate solution (20 ml). The aqueous phase was extracted further with ethyl acetate (2×20 ml), and the combined organic extracts dried (magnesium sulphate), filtered and evaporated to dryness to give crude product. Purification was by silica gel column chromatography [Ethyl acetate] to provide the target compound as an oil/solid.

δ_{H} (CDCl3; 250 MHz) 2.46 (3H, s, CH3), 2.58 (1H, ddd, J14.3, 6.1, 2.6, one of CH₂CH₂), 2.83 (1H, ddd, J14.3, 7.9, 2.1, one of CH₂CH₂), 3.63 (1H, d, J13.1, one of ArCH₂NR₂), 3.86 (1H, ddd, J12.6, 6.1, 2.1, one of CH₂CH₂), 4.16 (1H, ddd, J12.6, 7.9, 2.6, one of CH₂CH₂), 4.68 (1H, d, J13.1, one of ArCH₂NR₂), 5.80 (1H, s, CHOR), 6.84 (1H, d, J8.3, aromatic H), 7.21-7.27 (2H, m, aromatic H), 7.32-7.40 (2H, m, aromatic H), 7.55 (1H, dd, J7.8, 1.6, aromatic H), 8.52 (1H, dd, J4.6, 1.6, aromatic H), 8.54 (1H,d, J8.6, aromatic H).

### EXAMPLE 92

### 8Cyano-5-methyl-1-(3-pyridyl)-1,3,4,6-tetrahydro-5H-benz[f]-2,5-oxazocine

8-Bromo-5-methyl-1-(3-pyridyl)-1,3,4,6-tetrahydro-5H-benz[f]-2,5-oxazocine (50 mg, 0.15 mmol), Zn (1 mg, 0.01 mmol), Zn(OAc)₂ (4 mg, 0.02 mmol), Zn(CN)₂ (13 mg, 0.11 mmol), Pd₂ (dba)₃ (18 mg 0.02 mmol), and 1,1′-bis(diphenylphosphine)ferrocene (9 mg 0.02 mmol) were heated to 140° C. in DMF (2 ml) and H₂O (20 μl) overnight. The black reaction mixture was allowed to cool and diluted with water (20 ml). The resulting brown precipitate was collected by filtration and washed with water then brine. The filtrate was extracted with ethyl acetate (2×50 ml). The combined organic extracts were dried (magnesium sulphate), filtered and concentrated in vacuo to provide the crude product as a brown oil. Purification was by silica gel column chromatography [dichloromethane] to furnish the desired product (10 mg, 24%) as a yellow oil.

δ_{H}(CDCl3, 250 MHz) 2.47 (3H, s, CH₃), 2.58-2.84 (2H, m, CH₂CH₂), 3.68 (1H, d, J13.1, one of ArCH₂NR₂), 3.82-3.90 (1H, m, one of CH₂CH₂), 4.19-4.29 (1H, m, one of CH₂CH₂), 4.88 (1H, d, J13.1, one of ArCH₂NR₂), 5.84 (1H, s, CHOR), 7.11 (1H, d, J8.3, aromatic H), 7.22-7.30 (2H, m, aromatic H), 7.49-7.55 (3H, m, aromatic H), 8.52 (1H, dd, J4.6, 1.6 aromatic H), 8.55 (1H, d, J8.6, aromatic H).

### 2-Benzoyl-4-bromobenzoic acid and 2-benzoyl-5-bromobenzoic acid

Bromophthalic anhydride (40.0 g, 0.176 mol) was suspended in benzene (82.5 g, 1.06 mol) at RT under a nitrogen atmosphere. AlCl₃ (47 g, 0.35 mol) was added portionwise, the colour becoming more orange with each addition. After approximately ¾ of the AlCl₃ had been added, further additions produced violent reactions and thus the additions were slowed. The reaction mixture was then heated at reflux temperature for 5.5 hours before quenching into HCl/H₂O (1:9 mix, 1 L). A white precipitate formed immediately. The aqueous phase was extracted into ethyl acetate (2 L), dried (MgSO₄), filtered and concentrated in vacuo to give an off-white solid. Trituration with ethyl acetate, followed by a recrystallisation from EtOH/H₂O gave the target keto-acid as a mixture of 2 regio-isomers (19.2 g, 33%) in 2 batches.

δ_{H}(MeOD; 250 MHz) 7.41-7.57 (5H, m, ArH), 7.67-7.70 (2H, m, ArH), 7.79 (1H, dd, J8.3, 2.0, ArH), 7.97 (1H, d, J8.3, ArH).

### [5-Bromo-2-(thiazolidine-3-carbonyl)-phenyl]-phenylmethanone

A mixture of 2-benzoyl4-bromobenzoic acid and 2-benzoyl-5-bromobenzoic acid (17 g, 0.06 mol) was suspended in dichloromethane (125 ml) at RT under a nitrogen atmosphere. DMF (2 drops) and then oxalyl chloride (5.83 ml, 0.067 mol) were added dropwise and the resulting mixture was stirred at RT for 5 hours, at which point gas evolution had ceased. The mixture was concentrated in vacuo to provide the crude acid chloride. This material was dissolved in dichloromethane (150 ml) and added to a cooled (ice bath) solution of thiazolidine (4.8 ml, 0.061 mol) and triethylamine (8.50 ml, 0.061 mol) in dichloromethane (150 ml). The reaction mixture was stirred overnight at RT. The reaction was quenched by the addition of sat. aq. sodium bicarbonate (300 mL) and extracted into dichloromethane (2×250 ml). The organic extracts were dried (MgSO₄), filtered and concentrated in vacuo to provide the crude amide as a mixture of regioisomers. Upon standing, the mixture began to crystallise. The crystals were isolated and found to be the desired amide (12.816 g, 56%).

δ_{H} (CDCl₃; 250 MHz) 3.02 (2H, t, J6.3, two of thiazolidine H), 3.65-3.80 (2H, m, two of thiazolidine H), 4.38-4.54 (2H, m, two of thiazolidine H), 7.36 (1H, d, J8.0, ArH), 7.46-7.52 (2H, m, ArH), 7.58-7.78 (5H, m, ArH).

### (5-Bromo-2-[{(2-mercapto-ethyl)-methylamino}-methyl]-phenyl)-phenylmethanol

[5-Bromo-2-(thiazolidine-3-carbonyl)-phenyl]-phenylmethanone (12.0 g, 31.7 mmol) was dissolved in dry THF (120 ml) at RT under a nitrogen atmosphere. BH₃SMe₂ (2.0 M in THF, 63 ml, 0.127 mol) was added dropwise and the resulting solution stirred at RT overnight. The reaction was quenched by the careful addition of 6M HCl (74 ml) and the mixture was then heated at reflux temperature for 1 hour. The reaction mixture was then concentrated to remove the THF before partitioning between MTBE and H₂O. The aqueous phase was basified with 2M NaOH and extracted into ethyl acetate. The organic extracts were dried (MgSO₄), filtered and concentrated in vacuo to provide the crude thiol. Purification was achieved by repeated silica gel column chromatography.

δ_{H} (CDCl₃; 250 MHz) 2.17 (3H, s, CH₃), 2.62-2.79 (4H, m, CH₂CH₂), 3.29 (2H, d, J4.0, ArCH₂NR₂), 5.80 (1H, s, CHOH), 7.06 (1H, d, J8.5, ArH), 7.23-7.41 (7H, m ArH).

Over time, it became apparent (test with Ellman's reagent) that the thiol was oxidised to the corresponding disulfide (shown below).
Reduction of Disulfide

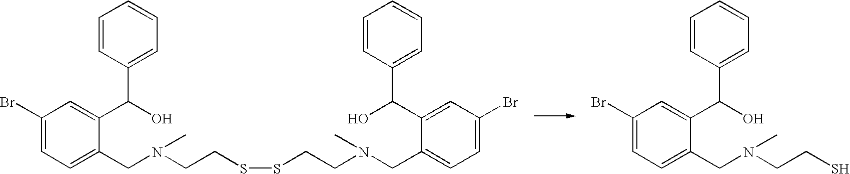

Disulfide (2.703 g, 3.7 mmol) was dissolved in dichloromethane (25 ml) at RT under nitrogen. Dithiothreitol (DTT) (628 mg, 4.07 mmol) was added and the resulting solution was stirred at RT overnight, during which time a precipitate formed. The mixture was diluted with dichlormethane and washed with water. The organic phase was separated and dried (MgSO₄), filtered then concentrated in vacuo to provide (5-bromo-2-[{(2-mercaptoethyl)-methylamino}methyl]-phenyl)-phenylmethanol (tested with Ellman's reagent) (2.4 g, 89%) which was used immediately in the next step.

### EXAMPLE 93

### [4-bromo-2-(phenylpropylsulfanylmethyl)-benzyl]-methylamine

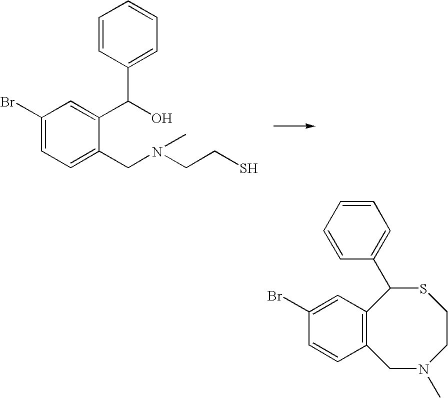

(5-Bromo-2-[{(2-mercaptoethyl)-methylamino}-methyl]-phenyl)-phenylmethanol (2.40 g, 6.56 mmol) was heated to reflux temperature under Dean-Stark conditions in toluene (25 ml) with pTSA (1.87 g, 9.84 mmol) under a nitrogen atmosphere, for 4 hours. The mixture was allowed to cool before diluting with ethyl acetate and washing with sat. aq. sodium bicarbonate solution. The organic extracts were dried (MgSO₄), filtered and concentrated in vacuo to provide the target compound as an impure oil. Purification by silica gel column chromatography [EtOAc] gave the product (584 mg, 26%).

δ_{H} (CDCl₃; 250 MHz) 2.28 (1H, dt, J15.9, 6.4, one of CH₂CH₂), 2.44 (3H, s, CH₃), 2.55 (1H, dt, J15.9, 4.6, one of CH₂CH₂), 3.00 (2H, t, J4.6, two of CH₂CH₂), 3.51 (1H, d, J14.3, one of ArCH₂NR₂), 4.08 (1H, d, J14.3, one of ArCH₂NR₂), 6.46 (1H, s, CHSH), 7.05-7.09 (2H, m, ArH), 7.25-7.40 (4H, ArH), 7.46-7.49 (2H, m, ArH).

### {4-Bromo-2[phenyl-(propane-1-sulfinyl)-methyl]-benzyl}-methylamine

[4-Bromo-2-(phenylpropylsulfanylmethyl)-benzyl]-methylamine (339 mg, 0.97 mmol) was dissolved in methanol (4 ml) and cooled in an ice bath. NaIO₄ (229 mg, 1.07 mmol) in water (3 ml) was added dropwise and the resulting mixture was stirred for 20 minutes in the ice bath and for 5 hours at RT. The reaction mixture was filtered and the filter cake washed with methanol. The filtrate was concentrated in vacuo to remove the solvent and as the mixture concentrated, a precipitate formed. Ethyl acetate was added to extract the crude product from the aqueous phase; the organic extracts were dried (MgSO₄), filtered and concentrated in vacuo to provide the crude material. Purification by gravity silica gel column chromatography [EtOAc] gave the product (35 mg, 10%) as a cream solid.

δ_{H} (CDCl₃; 250 MHz) 2.35 and 2.44 (2×3H, 2×s, CH₃ two diastereoisomers), 2.46-2.52 (2H, m, one of CH₂CH₂ for each diastereoisomer), 2.54-2.81 (2H, m, one of CH₂CH₂ for each diastereoisomer), 2.82-2.99 (4H, m, two of ArCH₂NCH₂CH₂ for each diastereoisomer), 3.59-3.76 (2H, m, one of ArCH₂NCH₂CH₂ for each diastereoisomer), 4.09-4.19 (2H, m, one of ArCH₂NCH₂CH₂ for each diastereoisomer), 6.35 and 6.38 (2×1H, 1×s, CHS), 7.15 (2H, dd, J7.9, 2.1, one ArH for each diastereoisomer), 7.24-7.27 (2H, m, one ArH for each diastereoisomer), 7.36-7.47, (8H, m, four of ArH for each diastereoisomer), 7.55 (2H, bd, J7.3, one of ArH for each diastereoisomer), 7.68 (2H, bd, J6.7, one of ArH for each diastereoisomer); m/z (ES⁺) 364 and 366 (MH⁺ for Br⁷⁹ and Br⁸¹).

The following Assays may be used to test the properties of compounds of the invention.

Biological Assay

The assay is carried out according to the method described by Perovic et al (1995), Arzneim-Forsch. Drug Res., 45: 1145.

Assay for Inhibition of Noradrenaline Reuptake Activity

Synaptosomes (100 μg) are incubated for 20 min at 37° C. with 0.1 μCi [³H]norepinephrine In the absence (control) or presence of the test compound or the reference compound in a buffer containing 118 mM NaCl, 5 mM KCl, 2.5 mM MgSO₄, 1.2 mM NaH₂PO₄, 25 mM NaHCO₃, 11 mM glucose, 10 μM EGTA and 50 μM ascorbic acid (pH 7.4). Basal control activity is determined by incubating the same mixture for 20 min at 0° C. in the presence of 10 μM protriptyline to block the uptake. Following incubation, the samples are filtered rapidly under vacuum through glass fiber filters (GF/B, Packard) and rinsed twice with ice-cold incubation buffer using a 96-sample cell harvester (Unifilter, Packard) to eliminate free [³H]norepinephrine. The filters are dried and the retained radioactivity is measured in a scintillation counter (Topcount, Packard) using a scintillation cocktail (Microscint 0, Packard).

The results are expressed as a percent inhibition of the control uptake of [³H]norepinephrine. The standard inhibitory reference compound is protriptyline, which is tested in each experiment at several concentrations to obtain an inhibition curve from which its IC₅₀ value is calculated.

Assay for Inhibition of Serotonin Reuptake Activity

Synaptosomes (100 μg) are incubated for 15 min at 37° C. with 0.1 μCi [³H]serotonin in the absence (control) or presence of the test compound or the reference compound in a buffer containing 118 mM NaCl, 5 mM KCl, 2.5 mM MgSO₄, 1.2 mM NaH₂PO₄, 25 mM NaHCO₃, 11 mM glucose, 10 μM EGTA and 50 μM ascorbic acid (pH 7.4).

Basal control activity is determined by incubating the same mixture for 15 min at 4° C. in the presence of 10 μM imipramine to block the uptake. Following incubation, the samples are filtered rapidly under vacuum through glass fiber filters (GF/B, Packard) and rinsed twice with ice-cold incubation buffer using a 96-sample cell harvester (Unifilter, Packard) to eliminate free [³H]serotonin. The filters are dried and the retained radioactivity is measured in a scintillation counter (Topcount, Packard) using a scintillation cocktail (Microscint 0, Packard).

The results are expressed as a percent inhibition of the control uptake of [³H]serotonin. The standard inhibitory reference compound is imipramine, which is tested in each experiment at several concentrations to obtain an inhibition curve from which its IC₅₀ value is calculated.

What is claimed is:

1. A compound of general formula (1A) 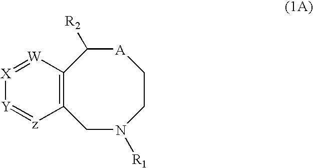
wherein R₁ is H, C₁-C₆ alkyl optionally substituted with F or C₃-C₈ cycloalkyl or C₂-C₄ alkenyl;
A is O, CH₂ or S(O)ₙ where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O)ₙ where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆alkyl, C₂-C₆alkenyl, C₂-C₆ alkynyl, C₃-C₆cycloalkyl, aryl and heteroaryl; and
R₅ is H, C₁-C₆alkyl, C₂-C₆alkenyl, C₂-C, alkynyl, C₃-C₆cycloalkyl, aryl or heteroaryl and is the same as or different to another R₅;
or a pharmaceutically acceptable salt thereof.

2. A compound of claim 1, wherein R₂ is optionally substituted heteroaryl, cycloalkyl or cycloalkenyl.

3. A compound of claim 1, wherein R₂ is optionally substituted phenyl.

4. A compound of claim 1, selected from:
5-benzyl-1-phenyl-1,3,4,6-tetrahydro-5H-benz[f]-2,5-oxazocine;
5-allyl-1-phenyl-1,3,4,6-tetrahydro-5H-benz[f]-2,5-oxazocine;
5-cyclopropyl-1-phenyl-1,3,4,6-tetrahydro-5H-benz[f]-2,5-oxazocine;
5-propyl-1-phenyl-1,3,4,6-tetrahydro-5H-benz[f]-2,5-oxazocine;
5-methylcyclopropyl-1-phenyl-1,3,4,6-tetrahydro-5H-benz[f]-2,5-oxazocine;
5-isopropyl-1-phenyl-1,3,4,6-tetrahydro-5H-benz[f]-2,5-oxazocine;
8-(4-pyridyl)-5-methyl-1-(3-methoxy)phenyl-1,3,4,6-tetrahydro-5H-benz[f]-2,5-oxazocine;
8-(5-pyrimidine)-5-methyl-1-(3-methoxy)phenyl-1,3,4,6-tetrahydro-5H-benz[f]-2,5-oxazocine;
5-methyl-1-(3-methoxy)phenyl-1,3,4,6-tetrahydro-5H-benz[f]-2,5-oxazocine;
5-methyl-1-(3-methoxy)phenyl-1,3,4,6-tetrahydro-5H-benz[f]-2,5-oxazocine-8-carboxamide;
10-fluoro-5-methyl-1-phenyl-1,3,4,6-tetrahydro-5H-benz[f]-2,5-oxazocine;
7-fluoro-5-methyl-1-phenyl-1,3,4,6-tetrahydro-5H-benz[f]-2,5-oxazocine;
8-bromo-5-methyl-1-(3-tosyloxy)phenyl-1,3,4,6-tetrahydro-5H-benz[f]-2,5-oxazocine;
8-bromo-5-methyl-1-(3-hydroxy)phenyl-1,3,4,6-tetrahydro-5H-benz[f]-2,5-oxazocine;
8-cyano-5-methyl-1-(3-hydroxy)phenyl-1,3,4,6-tetrahydro-5H-benz[f]-2,5-oxazocine;
8-cyclopropyl-5-methyl-1-(3-hydroxy)phenyl-1,3,4,6-tetrahydro-5H-benz[f]-2,5-oxazocine;
8-bromo-5-methyl-1(3-allyloxy)phenyl-1,3,4,6-tetrahydro-5H-benz[f]-2,5-oxazocine;
8-cyano-5-methyl-1-(3-allyloxy)phenyl-1,3,4,6-tetrahydro5H-benz[f]-2,5-oxazocine;
8-cyclopropyl-5-methyl-1-(3-allyloxy)phenyl-1,3,4,6-tetrahydro-H-benz[f]-2,5-oxazocine;
8-bromo-5-methyl-1-(2-methyl-3-methoxy)phenyl-1,3,4,6-tetrahydro-5H-benz[f]-2,5-oxazocine;
8-cyano-5-methyl-1-(2-methyl-3-methoxy)phenyl-1,3,4,6-tetrahydro-5H-benz[f]-2,5-oxazocine;
8-cyclopropyl-5-methyl-1-(2-methyl-3-methoxy)phenyl-1,3,4,6-tetrahydro-5H-benz[f]-2,5-oxazocine;
8-bromo-5-methyl-1-(3-methoxy-4-methyl)phenyl-1,3,4,6-tetrahydro-5H-benz[f]-2,5-oxazocine;
8-cyano-5-methyl-1-(3-methoxy-4-methyl)phenyl-1,3,4,6-tetrahydro-5H-benz[f]-2,5-oxazocine;
5-methyl-1-(3-methoxy-4-methyl)phenyl-1,3,4,6-tetrahydro-5H-benz[f]-2,5-oxazocine-8-carboxamide;
8cyclopropyl-5-methyl-1-(3-methoxy-4-methyl)phenyl-1,3,4,6-tetrahydro-5H-benz[f]-2,5-oxazocine;
9-bromo-5-methyl-1-(3,4-methylenedioxy)phenyl-1,3,4,6-tetrahydro-5H-benz[f]-2,5-oxazocine;
9-cyano-5-methyl-1-(3,4-methylenedioxy)phenyl-1,3,4,6-tetrahydro-5H-benz[f]-2,5-oxazocine;
9-cyclopropyl-5-methyl-1-(3,4-methylenedioxy)phenyl-1,3,4,6-tetrahydro-5H-benz[f]-2,5-oxazocine;
8-bromo-5-methyl-1-(3,4-methylenedioxy)phenyl-1,3,4,6-tetrahydro-5H-benz[f]-2,5-oxazocine;
8-cyano-5-methyl-1-(3,4-methylenedioxy)phenyl-1,3,4,6-tetrahydro-5H-benz[f]-2,5-oxazocine;
8-cyclopropyl-5-methyl-1-(3,4-methylenedioxy)phenyl-1,3,4,6-tetrahydro-5H-benz[f]-2,5-oxazocine;
8-bromo-5-methyl-1-(3-ethoxy)phenyl-1,3,4,6-tetrahydro-5H-benz[f]-2,5-oxazocine;
8-cyano-5-methyl-1-(3-ethoxy)phenyl-1,3,4,6-tetrahydro-5H-benz[f]-2,5-oxazocine;
8-cyclopropyl-5-methyl-1-3-ethoxy)phenyl-1,3,4,6-tetrahydro-5H-benz[f]-2,5-oxazocine;
9-bromo-5-methyl-1-(3-ethoxy)phenyl-1,3,4,6-tetrahydro-5H-benz[f]-2,5-oxazocine;
9-cyano-5-methyl-1-(3-ethoxy)phenyl-1,3,4,6-tetrahydro-5H-benz[f]-2,5-oxazocine;
9-cyclopropyl-5-methyl-1-(3-ethoxy)phenyl-1,3,4,6-tetrahydro-5H-benz[f]-2,5-oxazocine;
8-bromo-5-methyl-1-(3-trifluoromethoxy)phenyl-1,3,4,6-tetrahydro-5H-benz[f]-2,5-oxazocine;
8-cyano-5-methyl-1-(3-trifluoromethoxy)phenyl-1,3,4,6-tetrahydro-5H-benz[f]-2,5-oxazocine;
8-cyclopropyl-5-methyl-1-(3-trifluoromethoxy)phenyl-1,3,4,6-tetrahydro-5H-benz[f]-2,5-oxazocine;
9-bromo-5-methyl-1-(3-trifluoromethoxy)phenyl-1,3,4,6-tetrahydro-5H-benz[f]-2,5-oxazocine;
9-cyclopropyl-5-methyl-1-(3-trifluoromethoxy)phenyl-1,3,4,6-tetrahydro-5H-benz[f]-2,5-oxazocine;
9-bromo-5-methyl-1-(2-methyl)phenyl-1,3,4,6-tetrahydro-5H-benz[f]-2,5-oxazocine;
9-cyano-5-methyl-1-(2-methyl)phenyl-1,3,4,6-tetrahydro-5H-benz[f]-2,5-oxazocine;
9-cyclopropyl-5-methyl-1-(2-methyl)phenyl-1,3,4,6-tetrahydro-5H-benz[f]-2,5-oxazocine;
8-bromo-5-methyl-1-(2-methyl)phenyl-1,3,4,6-tetrahydro-5H-benz[f]-2,5-oxazocine;
8-cyano-5-methyl-1-(2-methyl)phenyl-1,3,4,6-tetrahydro-5H-benz[f]-2,5-oxazocine;
8-cyclopropyl-5-methyl-1-(2-methyl)phenyl-1,3,4,6-tetrahydro-5H-benz[f]-2,5oxazocine;
9-bromo-5-methyl-1-(3-pyridyl)-1,3,4,6-tetrahydro-5H-benz[f]-2,5-oxazocine;
8-bromo-5-methyl-1-(3-pyridyl)-1 3,4,6-tetrahydro-5H-benz[f]-2,5-oxazocine; and
8-cyano-5-methyl-1-(3-pyridyl)-1,3,4,6-tetrahydro-5H-benz[f]-2,5-oxazocine.

5. A compound of the general formula (1): 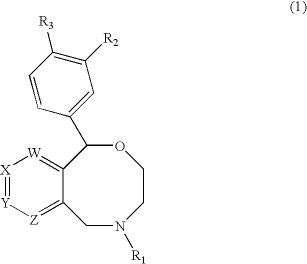
wherein
R₁ is H, C₁-C₆ alkyl, optionally substituted with F or _{c3}-C₆ cycloalkyl or C₂-C₆ alkenyl;
either R₂ and R₃ are the same or different and are H, a halogen, CN, CF₃, C₁-C₆ alkyl or OR₁, or R₂ and R₃ form a five or six membered ring which may be carbocyclic, heterocyclic (containing 1-2 heteroatoms taken from O, N or S), aromatic or heteroaromatic (containing 1-2 heteroatoms taken from O and N);
one of W, X, Y and Z is N, or CR₄ and the others are each CH;
R₄ is a halogen atom, CF₃, CN, OR₇, SO₂N(R₆)₂, COR₆, CO₂R₆, CON(R₆)₂, NR₁COR₅, NR₁SO₂R₅, NR₁CO₂R₅, NR₁CON(R₆)₂, OC₁-C₆ alkyl optionally substituted with R₄, C₁-C₆ alkyl optionally substituted with R₄, C₃-C₆ cycloalkyl optionally substituted with R₄, C₂-C₆ alkenyl optionally substituted with R₄, C₂-C₆ alkynyl optionally substituted with R₄, aryl optionally substituted with R₄, or a five or six membered aromatic heterocycle containing 1-4 heteroatoms selected from N and O, linked either through carbon or nitrogen;
R₅ is C₁-C₆ alkyl, C₂-C₆ alkenyl, C₂-C₆ alkynyl, C₃-C₆ cycloalkyl, aryl or heteroaryl;
each R₆ (which may be the same or different) is H, C₁-C₆ alkyl, C₂-C₆ alkenyl, C₂-C₆ alkynyl, C₃-C₆ cycloalkyl, aryl or heteroaryl; and
R₇ is aryl or heteroaryl;
or a pharmaceutically acceptable salt thereof.

6. A compound of claim 5, wherein R₄is CN, OR₇, SO₂N(R₆)₂, COR₆, CO₂R₆, CON(R₆)₂, NR₁COR₅, NR₁SO₂R₅, NR₁CON(R₆)₂, OC₁-C₆alkyl substituted with R₄, C₁-C₆ alkyl substituted with R₄, C₃-C₆ cycloalkyl optionally substituted with R₄, C₂-C₆ alkenyl optionally substituted with R₄, C₂-C₆ alkynyl optionally substituted with R₄, aryl optionally substituted with R₄, or a five or six-membered aromatic heterocycle containing 1-4 heteroatoms selected from N and O, linked either through carbon or nitrogen.

7. A compound or claim 6, wherein R₄ is CN, CON(R₆)₂, optionally substituted cycloalkyl or aryl, or a five or six-membered aromatic heterocycle.

8. A compound of claim 5, wherein R₂ is halogen, CN, CF₃, C₁-C₆ alkyl or OR₁.

9. A compound of claim 5, wherein R₂ and R₃ form a ring.

10. A compound of claim 8, wherein R₂ is OR₁.

11. A compound of claim 5, selected from
N-(2-hydroxyethyl)-N-methyl-4-bromo-2-(1-hydroxy-1-phenyl)methyl benzylamine (4a)
N-(2-hydroxyethyl)-N-methyl-5-bromo-2-(1-hydroxy-1-phenyl)methyl benzylamine (4b)
9-bromo-5-methyl-1-phenyl-1,3,4,6-tetrahydro-5H-benz[f]-2,5-oxazocine (5a)
8-bromo-5-methyl-1-phenyl-1,3,4,6-tetrahydro-5H-benz[f]-2,5-oxazocine (5b)
9-bromo-5-methyl-1-(4-methoxy)phenyl-1,3,4,6-tetrahydro-5H-benz[f]-2,5-oxazocine (17a)
8-bromo-5-methyl-1-(4-methoxy)phenyl-1,3,4,6-tetrahydro-5H-benz[f]-2,5-oxazocine (17b)
9-bromo-5-methyl-1-(3-methoxy)phenyl-1,3,4,6-tetrahydro-5H-benz[f]-2,5-oxazocine (21a)
8-bromo-5-methyl-1-(3-methoxy)phenyl-1,3,4,6-tetrahydro-5H-benz[f]-2,5-oxazocine (21b)
9-methoxymethyl-1-(3-methoxy)phenyl-1,3,4,6-tetrahydro-5H-benz[f]-2,5-oxazocine (24a)
8-methoxy-5-methyl-1-(3-methoxy)phenyl-1,3,4,8-tetrahydro-5H-benz[f]-2,5-oxazocine (24b)
9-methoxy-5-methyl-1-(4-methoxy)phenyl-1,3,4,6-tetrahydro-5H-benz[f]-2,5-oxazocine (31a) and
8-methoxy-5-methyl-1-(3-methoxy)phenyl-1,3,4,6-tetrahydro-5H-benz[f]-2,5-oxazocine (31b).

12. A compound of claim 5, selected from
9-cyano-5-methyl-1-phenyl-1,3,4,6-tetrahydro-5H-benz[f]-2,5-oxazocine (6a)
8-cyano-5-methyl-1-phenyl-1,3,4,6-tetrahydro-5H-benz[f]-2,5-oxazocine (6b)
5-methyl-1-phenyl-1,3,4,6-tetrahydro-5H-benz[f]-2,5-oxazocine (7a)
5-methyl-1-phenyl-1,3,4,6-tetrahydro-5H-benz[f]-2,5-oxazocine-8-carboxamide (7b)
N-(1,1,1-trimethylmethoxycarbonyl)-5-methyl-1-phenyl-1,3,4,6-tetrahydro-5H-benz[f]-2,5-oxazocine-9-methylamine (8a)
N-(1,1 1-trimethylmethoxycarbonyl)-5-methyl-1-phenyl-1,3,4,6-tetrahydro-5H-benz[f]-2,5-oxazocine-8-methylamine (8b)
5-methyl-1,9-diphenyl-1,3,4,6-tetrahydro-5H-benz[f]-2,5-oxazocine (9a)
5-methyl-1,8-diphenyl-1,3,4,6-tetrahydro-5H-benz[f]-2,5-oxazocine (9b)
9-(3,5-dimethylisoxazol-4-yl)-5-methyl-1-phenyl-1,3,4,6-tetrahydro-5H-benz[f]-2,5-oxazocine (10a)
8-(3,5-dimethylisoxazol-4-yl)-5-methyl-1-phenyl-1,3,4,6-tetrahydro-5H-benz[f]-2,5-oxazocine (10b)
2-(5-methyl-1-phenyl-1,3,4,6-tetrahydro-5H-benz[f]-2,5-oxazocine-9-ethenyl)carboxamide (11a)
2-(5-methyl-1-phenyl-1,3,4,6-tetrahydro-5H-benz[f]-2,5-oxazocine-8-ethenyl)carboxamide (11b)
2-(5-methyl-1-phenyl-1,3,4,6-tetrahydro-5H-benz[f]-2,5-oxazocine-9-ethyl)carboxamide (12a)
2-(5-methyl-1-phenyl-1,3,4,6-tetrahydro-5H-benz[f-2,5-oxazocine-8-ethyl)carboxamide (12b)
5-methyl-1-phenyl-1,3,4,6-tetrahydro-5H-benz[f]-2,-oxazocine-9-methylamine (13a)
5-methyl-1-phenyl-1,3,4,6-tetrahydro-5H-benz[f]-2,5-oxazocine-8-methylamine (13b)
9-cyano-5-methyl-1-(3-methoxy)phenyl-1,3,4,6-tetrahydro-5H-benz[f]-2,5-oxazocine (22a)
8-cyano-5-methyl-1-(3-methoxy)phenyl-1,3,4,6-tetrahydro-5H-benz[f]-2,5-oxazocine (22b)
9-cyclopropyl-5-methyl-1-phenyl-1,3,4,6-tetrahydro-5H-benz[f]-2,5-oxazocine (26a)
8-cyclopropyl-5-methyl-1-phenyl-1,3,4,6-tetrahydro-5H-benz[f]-2,5-oxazocine (26b)
N-(acetyl)-5-methyl-1-phenyl-1,3,4,6-tetrahydro-5H-benz[f]-2,5-oxazocine-9-methylamine (27a)
N-(acetyl)-5-methyl-1-phenyl-1,3,4,6-tetrahydro-5H-benz[f]-2,5-oxazocine-8-methylamine (27b)
9-cyclopropyl-5-methyl-1-(3-methoxy)phenyl-1,3,4,6-tetrahydro-5H-benz[f]-2,5-oxazocine (29a)
8-cyclopropyl-5-methyl-1-(3-methoxy)phenyl-1,3,4,6-tetrahydro-5H-benz[f]-2,5-oxazocine (29b)
9-cyclopropyl-5-methyl-1-(4-methoxy)phenyl-1, 3,4,6-tetrahydro-5H-benz[f]-2,5-oxazocine (30a)
8-cyclopropyl-5-methyl-1-(4-methoxy)phenyl-1,3,4,6-tetrahydro-5H-benz[f]-2,5-oxazocine (30b) and
9-cyano-5-methyl-1-(4-methoxy)phenyl-1,3,4,6-tetrahydro-5H-benz[f]-2,5-oxazocine (32a).

13. A compound of general formula (1B) 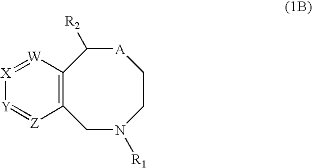
wherein R₁ is H, C₁-C₆ alkyl optionally substituted with F or C₃-C₆ cycloalkyl or C₂-C₄ alkenyl;
A is O, CH₂ or S(O)ₙ where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O), where n is 0-2, and optionally substituted with R₃; or a phenyl group optionally substituted In one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R)₂; OC₁-C₆ alkyl substituted with R₃; C₁-C₆ alkyl optionally substituted with unsubstituted R₃; C₃-C₅ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆ alkyl, C₂-C₆ alkenyl, C₂-C₆ alkynyl, C₃-C₆ cycloalkyl, aryl and heteroaryl; and
R₅ is H, C₁-C₆alkyl, C₂-C₆alkenyl, C₂-C₆ alkynyl, C₃-C₆cycloalkyl, aryl or heteroaryl and is the same as or different to another R₅;
or a pharmaceutically acceptable salt thereof.

14. A compound of claim 13, wherein R₂ is optionally substituted heteroaryl, cycloalkyl or cycloalkenyl.

15. A compound of claim 13, wherein R₂ is optionally substituted phenyl.

16. A compound of claim 13, selected from:
5-benzyl-1-phenyl-1,3,4,6-tetrahydro-5H-benz[f]-2,5-oxazocine;
5-allyl-1-phenyl-1,3,4,6-tetrahydro-5H-benz[f]-2,5-oxazocine;
5-cyclopropyl-1-phenyl-1,3,4,6-tetrahydro-5H-benz[f]-2,5-oxazocine;
5-propyl-1-phenyl-1,3,4,6-tetrahydro-5H-benz[f]-2,5-oxazocine;
5-methylcyclopropyl-1-phenyl-1,3,4,6-tetrahydro-5H-benz[f]-2,5-oxazocine;
5-isopropyl-1-phenyl-1,3,4,6-tetrahydro-5H-benz[f]-2,5-oxazocine;
8-(4-pyridyl)-5-methyl-1-(3-methoxy)phenyl-1,3,4,6-tetrahydro-5H-benz[f]-2,5-oxazocine;
8-(5-pyrimidine)-5-methyl-1-(3-methoxy)phenyl-1,3,4,6-tetrahydro-5H-benz[f]-2,5-oxazocine;
5-methyl-1-(3-methoxy)phenyl-1,3,4,6-tetrahydro-5H-benz[f]-2,5-oxazocine;
5-methyl-1-(3-methoxy)phenyl-1,3,4,6-tetrahydro-5H-benz[f]-2,5oxazocine-8-carboxamide;
10-fluoro-5-methyl-1-phenyl-1,3,4,6-tetrahydro-5H-benz[f]-2,5-oxazocine;
7-fluoro-5-methyl-1-phenyl-1,3,4,6-tetrahydro-5H-benz[f]-2,5-oxazocine;
8-bromo-5-methyl-1-(3-tosyloxy)phenyl-1,3,4,6-tetrahydro-5H-benz[f]-2,5-oxazocine;
8-bromo-5-methyl-1-(3-hydroxy)phenyl-1,3,4,6-tetrahydro-5H-benz[f]-2,5-oxazocine;
8-cyano-5-methyl-1-(3-hydroxy)phenyl-1,3,4,6-tetrahydro-5H-benz[f]-2,5-oxazocine;
8-cyclopropyl-5-methyl-1-(3-hydroxy)phenyl-1,3,4,6-tetrahydro-5H-benz[f]-2,5-oxazocine;
8-bromo-5-methyl-1-(3-allyloxy)phenyl-1,3,4,6-tetrahydro-5H-benz[f]-2,5-oxazocine;
8-cyano-5-methyl-1-(3-allyloxy)phenyl-1,3,4,6-tetrahydro-5H-benz[f]-2,5-oxazocine;
8-cyclopropyl-5-methyl-1-(3-allyloxy)phenyl-1,3,4,6-tetrahydro-5H-benz[f]-2,5-oxazocine;
8-bromo-5-methyl-1-(2-methyl-3-methoxy)phenyl-1,3,4,6-tetrahydro-5H-benz[f]-2,5-oxazocine;
8-cyano-5-methyl-1-(2-methyl-3-methoxy)phenyl-1,3,4,6-tetrahydro-5H-benz[f]-2,5-oxazocine;
8-cyclopropyl-5-methyl-1-(2-methyl-3-methoxy)phenyl-1,3,4,6-tetrahydro-5H-benz[f]-2,5-oxazocine;
8-bromo-5-methyl-1-(3-methoxy-4-methyl)phenyl-1,3,4,6-tetrahydro-5H-benz[f]-2,5-oxazocine;
8-cyano-5-methyl-1-(3-methoxy-4-methyl)phenyl-1,3,4,6-tetrahydro-5H-benz[f]-2,5-oxazocine;
5-methyl-1-3-methoxy-4-methyl)phenyl-1,3,4,6-tetrahydro-5H-benz[f]-2,5-oxazocine-8-carboxamide;
8-cyclopropyl-5-methyl-1-(3-methoxy-4-methyl)phenyl-1,3,4,6-tetrahydro-5H-benz[f]-2,5-oxazocine;
9-bromo-5-methyl-1-(3,4-methylenedioxy)phenyl-1,3,4,6-tetrahydro-5H-benz[f]-2,5-oxazocine;
9-cyano-5-methyl-1-(3,4-methylenedioxy)phenyl-1,3,4,6-tetrahydro-5H-benz[f]-2,5-oxazocine;
9-cyclopropyl-5-methyl-1-(3,4-methylenedioxy)phenyl-1,3,4,6-tetrahydro-5H-benz[f]-2,5-oxazocine;
8-bromo-5-methyl-1-(3,4-methylenedioxy)phenyl-1,3,4,6-tetrahydro-5H-benz[f]-2,5oxazocine;
8-cyano-5-methyl-1-(3,4-methylenedioxy)phenyl-1,3,4,6-tetrahydro-5H-benz[f]-2,5-oxazocine;
8-cyclopropyl-5-methyl-1-(3,4-methylenedioxy)phenyl-1,3,4,6-tetrahydro-5H-benz[f]-2,5-oxazocine;
8-bromo-5-methyl-1-(3-ethoxy)phenyl-1,3,4,6-tetrahydro-5H-benz[f]-2,5-oxazocine;
8-cyano-5-methyl-1-(3-ethoxy)phenyl-1,3,4,6-tetrahydro-5H-benz[f]-2,5-oxazocine;
8-cyclopropylmethyl-1-(3-ethoxy)phenyl-1,3,4,6-tetrahydro-5H-benz[f]-2,5-oxazocine;
9-bromo-5-methyl-1-(3-ethoxy)phenyl-1,3,4,6-tetrahydro-5H-benz[f]-2,5-oxazocine;
9-cyano-5-methyl-1-(3-ethoxy)phenyl-1,3,4,6-tetrahydro-5H-benz[f]-2,5-oxazocine;
9-cyclopropyl-5-methyl-1-(3-ethoxy)phenyl-1,3,4,6-tetrahydro-5H-benz[f]-2,5-oxazocine;
8-bromo-5-methyl-1-(3-trifluoromethoxy)phenyl-1,3,4,6-tetrahydro-5H-benz[f]-2,5-oxazocine;
8-cyano-5-methyl-1-(3-trifluoromethoxy)phenyl-1,3,4,6-tetrahydro-5H-benz[f]-2,5-oxazocine;
8-cyclopropyl-5-methyl-1-(3-trifluoromethoxy)phenyl-1,3,4,6-tetrahydro-5H-benz[f]-2,5-oxazocine;
9-bromo-5-methyl-1-(3-trifluoromethoxy)phenyl-1,3,4,6-tetrahydro-5H-benz[f]-2,5-oxazocine;
9-cyclopropyl-5-methyl-1-(3-trifluoromethoxy)phenyl-1,3,4,6-tetrahydro-5H-benz[f]-2,5-oxazocine;
9-bromo-5-methyl-1-(2-methyl)phenyl-1,3,4,6-tetrahydro-5H-benz[f]-2,5-oxazocine;
9-cyano-5-methyl-1-(2-methyl)phenyl-1,3,4,6-tetrahydro-5H-benz[f]-2,5-oxazocine;
9-cyclopropyl-5-methyl-1-(2-methyl)phenyl-1,3,4,6-tetrahydro-5H-benz[f]-2,5-oxazocine;
8-bromo-5-methyl-1-(2-methyl)phenyl-1,3,4,6-tetrahydro-5H-benz[f]-2,5-oxazocine;
8-cyano-5-methyl-1-(2-methyl)phenyl-1,3,4,6-tetrahydro-5H-benz[f]-2,5-oxazocine;
8-cyclopropyl-5-methyl-1-(2-methyl)phenyl-1,3,4,6-tetrahydro-5H-benz[f]-2,5-oxazocine;
9-bromo-5-methyl-1-(3-pyridyl)-1,3,4,6-tetrahydro-5H-benz[f]-2,5-oxazocine;
8-bromo-5-methyl-1-(3-pyridyl)-1,3,4,6-tetrahydro-5H-benz[f]-2,5-oxazocine; and
8-cyano-5-methyl-1-(3pyridyl)-1,3,4,6-tetrahydro-5H-benz[f]-2,5-oxazocine.

17. A compound of general formula (1C) 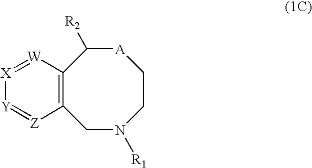
wherein R₁ is H, C₁-C₆ alkyl optionally substituted with F or C₃-C₆ cycloalkyl or C₂-C₄ alkenyl;
A is O, CH₂ or S(O)n where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O), where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆ alkyl, C₂-C₆ alkenyl, C₂-C₆ alkynyl, C₃-C₆ cycloalkyl, aryl and heteroaryl; and
R₅ is H, C₁-C₆alkyl, C₂-C₆alkenyl, C₂-C₆ alkynyl, C₃-C₆cycloalkyl, aryl or heteroaryl and is the same as or different to another R₅;
or a pharmaceutically acceptable salt thereof.

18. A compound of claim 17, wherein R₂ is optionally substituted heteroaryl, cycloalkyl or cycloalkenyl.

19. A compound of claim 17, wherein R₂ is optionally substituted phenyl.

20. A compound of claim 17, selected from:
5-benzyl-1-phenyl-1,3,4,6-tetrahydro-5H-benz[f]-2,5-oxazocine;
5-allyl-1-phenyl-1,3,4,6-tetrahydro-5H-benz-[f]-2,5-oxazocine;
5-cyclopropyl-1-phenyl-1,3,4,6-tetrahydro-5H-benz[f]-2,5-oxazocine;
5-propyl-1-phenyl-1,3,4,6-tetrahydro-5H-benz[f]-2,5-oxazocine;
5-methylcyclopropyl-1-phenyl-1,3,4,6-tetrahydro-5H-benz[f]-2,5-oxazocine;
5-isopropyl-1-phenyl-1,3,4,6-tetrahydro-5H-benz[f]-2,5-oxazocine;
8-(4-pyridyl)-5-methyl-1-(3-methoxy)phenyl-1,3,4,6-tetrahydro-5H-benz[f]-2,5-oxazocine;
8-(5-pyrimidine)-5-methyl-1-(3-methoxy)phenyl-1,3,4,6-tetrahydro-5H-benz[f]-2,5-oxazocine;
5-methyl-1-(3-methoxy)phenyl-1,3,4,6-tetrahydro-5H-benz[f]-2,5-oxazocine;
5-methyl-1 43-methoxy)phenyl-1,3,4,6-tetrahydro-5H-benz[f]-2,5-oxazocine-8-carboxamide;
10-fluoro-5-methyl-1-phenyl-1,3,4,6-tetrahydro-5H-benz[f]-2,5-oxazocine;
7-fluoro-5-methyl-1-phenyl-1,3,4,6-tetrahydro-5H-benz[f]-2,5-oxazocine;
8-bromo-5-methyl-1-(3-tosyloxy)phenyl-1,3,4,6-tetrahydro-5H-benz[f]-2,5-oxazocine;
8-bromo-5-methyl-1-(3-hydroxy)phenyl-1,3,4,6-tetrahydro-5H-benz[f]-2,5-oxazocine;
8-cyano-5-methyl-1-(3-hydroxy)phenyl-1,3,4,6-tetrahydro-5H-benz[f]-2,5-oxazocine;
8-cyclopropyl-5-methyl-1-(3-hydroxy)phenyl-1,3,4,6-tetrahydro-5H-benz[f]-2,5-oxazocine;
8-bromo-5-methyl-1-(3-allyloxy)phenyl-1,3,4,6-tetrahydro-5H-benz[f]-2,5-oxazocine;
8-cyano-5-methyl-1-(3-allyloxy)phenyl-1,3,4,6-tetrahydro-5H-benz[f]-2,5-oxazocine;
8-cyclopropyl-5-methyl-1-(3-alkyloxy)phenyl-1,3,4,6-tetrahydro-5H-benz[f]-2,5-oxazocine;
8-bromo-5-methyl-1-(2-methyl-3-methoxy)phenyl-1,3,4,6-tetrahydro-5H-benz[f]-2,5-oxazocine;
8-cyano-5-methyl-1-(2-methyl-3-methoxy)phenyl -1,3,4,6-tetrahydro-5H -benz[f]-2,5-oxazocine;
8-cyclopropyl-5-methyl-1-(2-methyl-3-methoxy)phenyl-1,3,4,6-tetrahydro-5H-benz[f]-2,5-oxazocine;
8-bromo-5-methyl-1-(3-methoxy-4-methyl)phenyl-1,3,4,6-tetrahydro-5H-benz[f]-2,5-oxazocine;
8-cyano-5-methyl-1-(3-methoxy-4-methyl)phenyl-1,3,4,6-tetrahydro-5H-benz[f]-2,5-oxazocine;
5-methyl-1-(3-methoxy4-methyl)phenyl-1,3,4,6-tetrahydro-5H-benz[f]-2,5-oxazocine-8-carboxamide;
8-cyclopropyl-5-methyl-1-(3-methoxy-4-methyl)phenyl-1,3,4,6-tetrahydro-5H-benz[f]-2,5-oxazocine;
9-bromo-5-methyl-1-(3,4-methylenedioxy)phenyl-1,3,4,6-tetrahydro-5H-benz[f]-2,5-oxazocine;
9-cyano-5-methyl-1-(3,4-methylenedioxy)phenyl-1,3,4,6-tetrahydro-5H-benz[f]-2,5-oxazocine;
9-cyclopropyl-5-methyl-1-(3,4-methylenedioxy)phenyl-1,3,4,6-tetrahydro-5H-benz[f]-2.5-oxazocine;
8-bromo-5-methyl-1-(3,4-methylenedioxy)phenyl-1,3,4,6-tetrahydro-5H-benz[f]-2,5-oxazocine;
8-cyano-5-methyl-1-(3,4-methylenedioxy)phenyl-1,3,4,6-tetrahydro-5H-benz[f]-2,5-oxazocine;
8-cyclopropyl-5-methyl-1-(3,4-methylenedioxy)phenyl-1,3,4,6-tetrahydro-5H-benz[f]-2,5-oxazocine;
8-bromo-5-methyl-1-(3-ethoxy)phenyl-1,3,4,6-tetrahydro-5H-benz[f]-2,5-oxazocine;
8-cyano-5-methyl-1-(3-ethoxy)phenyl-1,3,4,6-tetrahydro-5H-benz[f]-2,5-oxazocine;
8-cyclopropyl-5-methyl-1-(3-ethoxy)phenyl-1,3,4,6-tetrahydro-5H-benz[f]-2,5-oxazocine;
9-bromo-5-methyl-1-(3-ethoxy)phenyl-1,3,4,6-tetrahydro-5H-benz[f]-2,5-oxazocine;
9-cyano-5-methyl-1-(3-ethoxy)phenyl-1,3,4,6-tetrahydro-5H-benz[f]-2,5-oxazocine;
9-cyclopropyl-5-methyl-1-(3-ethoxy)phenyl-1,3,4,6-tetrahydro-5H-benz[f]-2,5-oxazocine;
8-bromo-5-methyl-1-(3-trifluoromethoxy)phenyl-1,3,4,6-tetrahydro-5H-benz[f]-2,5-oxazocine;
8-cyano-5-methyl-1-(3-trifluoromethoxy)phenyl-1,3,4,6-tetrahydro-5H-benz[f]-2,5-oxazocine;
8-cyclopropyl-5-methyl-1-(3-trifluoromethoxy)phenyl-1,3,4,6-tetrahydro-5H-benz[f]-2,5-oxazocine;
9-bromo-5-methyl-1-(3-trifluoromethoxy)phenyl-1,3,4,6-tetrahydro-5H-benz[f9-2,5-oxazocine;
9-cyclopropyl-5-methyl-1-(3-trifluoromethoxy)phenyl-1,3,4,6-tetrahydro-5H-benz[f]-2,5-oxazocine;
9-bromo-5-methyl-1-(2-methyl)phenyl-1,3,4,6-tetrahydro-5H-benz[f]-2,5-oxazocine;
9-cyano-5-methyl-1-(2-methyl)phenyl-1,3,4,6-tetrahydro-5H-benz[f]-2,5-oxazocine;
9-cyclopropyl-5-methyl-1-(2-methyl)phenyl-1,3,4,6-tetrahydro-5H-benz[f]-2,5-oxazocine;
8-bromo-5-methyl-1-(2-methyl)phenyl-1,3,4,6-tetrahydro-5H-benz[f]-2,5-oxazocine;
8-cyano-5-methyl-1-(2-methyl)phenyl-1,3,4,6-tetrahydro-5H-benz[f]-2,5-oxazocine;
8-cyclopropyl-5-methyl-1-(2-methyl)phenyl-1,3,4,6-tetrahydro-5H-benz[f]-2,5-oxazocine;
9-bromo-5-methyl-1-(3-pyridyl)-1,3,4,6-tetrahydro-5H-benz[f]-2,5-oxazocine;
8-bromo-5-methyl-1-(3-pyridyl)-1,3,4,6-tetrahydro-5H-benz[f]-2,5-oxazocine; and
8-cyano-5-methyl-1-(3-pyridyl)-1,3,4,6-tetrahydro-5H-benz[f]-2,5-oxazocine

21. A compound of general formula 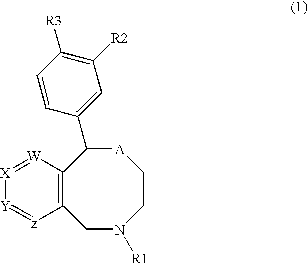
wherein:
R₁ is H, C₁-C₆alkyl, optionally substituted with F or C₃-C₆ cycloalkyl or C₂-C₄ alkenyl;
either R₂ and R₃ are the same or different and are each H, halogen, CN, CF₃, C₁-C₆ alkyl or OR₁, or R₂ and R₃ may form a five or six membered ring which may be carbocyclic, heterocyclic (containing 1-2 heteroatoms taken from O, N and S), aromatic or heteroaromatic (containing 1-2 heteroatoms taken from O and N); and
one of W, X, Y and Z is N, CH or CR₄ and the others are CH;
R₄ is halogen; CF₃; CN; OR₇; SO₂N(R₆)₂ (where each R₆ is the same or different); COR₆; CO₂R₆; CON(R₆)₂ (where R₆ is the same or different); NR₁COR₅; NR₁SO₂R₅; NR₁CO₂R₅; NR₁CON(R₆)₂ (where each R₆ is the same or different), OC₁-C₆ alkyl substituted with unsubstituted R₄, C₁-C₆ alkyl optionally substituted with unsubstituted R₄, C₃-C₆ cycloalkyl optionally substituted with unsubstituted R₄, C₂-C₆ alkenyl optionally substituted with unsubstituted R₄, C₂-C₆ alkynyl optionally substituted with unsubstituted R₄ and aryl optionally substituted with unsubstituted R₄, or R₄ is a five or six membered aromatic heterocycle containing 1-4 heteroatoms taken from N and O;
R₅ is C₁-C₆alkyl, C₂-C₆alkenyl, C₂-C₆ alkynyl, C₃-C₆cycloalkyl, aryl and heteroaryl;
R₆ can be H, C₁-C₆alkyl, C₂-C₆alkenyl, C₂-C₆ alkynyl, C₃-C₆cycloalkyl, aryl and heteroaryl; and
R₇ is aryl or heteroaryl;
or a pharmaceutically acceptable salt thereof.

22. A method for the treatment of a patient having a condition selected from depression, post-traumatic stress disorders, attention-deficit disorders, obsessive compulsive disorders, pre-menstrual syndrome, substance abuse, micturition disorders, sexual dysfunction, acute, chronic or neuropathic pain, dysmennorhoea, migraine headache, and emesis, which comprises administering to the patient an effective amount of a compound of claim 1.

23. The method of claim 22, wherein the condition is emesis.

24. The method of claim 23, wherein the emesis is acute, delayed, post-operative, last-phase, and anticipatory emesis.

25. The method of claim 23, wherein the emesis is induced by chemotherapy, radiation, toxins, pregnancy, vestibtilar disorder, motion, post-operative sickness, surgery, gastrointestinal obstruction, reduced gastrointestinal motility, visceral pain, migraine or opioid analgesic.

26. The method of claim 22, wherein the compound is selected from 9-methoxy-5-methyl-1-phenyl-1,3,4,6-tetrahydro-5H-benz[f]-2,5-oxazocine (23a) and 8-methoxy-5-methyl-1-phenyl-1,3,4,6-tetrahydro-5H-benz[f]-2,5-oxazocine (23b).
